(19) United States
(12) Patent Application Publication
(10) Pub. No.: US 2024/0034778 A1
(43) Pub. Date: Feb. 1, 2024

(54) AMYLOID-SPECIFIC ANTIBODIES AND USES THEREOF

(71) Applicant: THE REGENTS OF THE UNIVERSITY OF MICHIGAN, Ann Arbor, MI (US)

(72) Inventors: Alec A. Desai, Ann Arbor, MI (US); Peter M. Tessier, Ann Arbor, MI (US); Matthew D. Smith, Ann Arbor, MI (US); Yulei Zhang, Ann Arbor, MI (US)

(21) Appl. No.: 18/258,758

(22) PCT Filed: Dec. 21, 2021
(86) PCT No.: PCT/US21/64649

§ 371 Date: Jun. 21, 2023

Related U.S. Application Data
(60) Provisional application No. 63/129,318, filed on Dec. 22, 2020.

(51) Int. Cl. C07K 16/18; C12N 15/63; A61P 25/28; G01N 33/68

(52) U.S. Cl. CPC C07K 16/18; C12N 15/63; A61P 25/28; G01N 33/6896; C07K 2317/565; C07K 2317/52; G01N 2800/2821

(57) ABSTRACT

The disclosure is directed to a monoclonal antibody. or an antigen-binding fragment thereof. directed against fibrils of amyloid beta (Aβ) peptides, as well as a method of treating and diagnosing neurodegenerative diseases using the monoclonal antibody.

### CROSS-REFERENCE TO RELATED APPLICATIONS

This application claims the benefit of U.S. Provisional Application No. 63/129,318 filed Dec. 22, 2020, the content of which is herein incorporated by reference in its entirety.

### STATEMENT REGARDING FEDERALLY SPONSORED RESEARCH AND DEVELOPMENT

This invention was made with government support under GM104130 and GM136300 awarded by the National Institutes of Health. The government has certain rights in the invention.

### SEQUENCE LISTING STATEMENT

The text of the computer readable sequence listing filed herewith, titled “38670-601_SEQUENCE LISTING_ST25”, created Dec. 21, 2021, having a file size of 84,060 bytes, is hereby incorporated by reference in its entirety.

### FIELD

The disclosure provides conformational monoclonal antibodies directed against fibrils of amyloid beta (Aβ) peptides.

### BACKGROUND OF THE INVENTION

Of the many human disorders facing society today, neurodegenerative diseases such as Alzheimer's and Parkinson's diseases are arguably the most menacing and least treatable (1,2). These diseases, which are linked to the formation of toxic prefibrillar oligomers and amyloid fibrils, are particularly concerning because their frequency of occurrence is linked to age and, thus, the number of cases is expected to increase as life expectancy is increased in the coming years due to significant advances in treating other human disorders such as cancer and heart disease.

Conformational antibodies specific for different conformers of amyloid-forming proteins are important for detecting, disrupting, and reversing toxic protein aggregation (3). Several previous reports demonstrate methods for using immunization (4,5), directed evolution (6,7), and rational design methods for generating these antibodies (8). Despite this progress, there are several common problems associated with generating conformational antibodies against amyloid-forming proteins. These challenges typically result in antibodies that recognize protein aggregates with either conformational specificity (e.g., common fibril structure) (5) or sequence specificity (e.g., linear peptide epitopes), but not both. Even in the cases where antibodies with strict conformational and sequence specificity have been identified (9), these approaches typically require extensive secondary screening to identify such rare variants and are not readily extendable to generate conformational antibodies against different sites in the same protein or other proteins in a systematic, efficient, and predictable manner.

There remains a need for antibodies and methods of use to improve the treatment and diagnosis of neurodegenerative diseases characterized by the aggregation of amyloid-forming peptides, such as Alzheimer's disease.

### BRIEF SUMMARY OF THE INVENTION

The disclosure provides a monoclonal antibody directed against fibrils of amyloid beta (Aβ) peptides, or an antigen-binding fragment thereof, comprising a heavy chain variable region (VH) comprising complementarity determining regions (CDRs) HCDR1, HCDR2, and HCDR3 and a light chain variable region (VL) comprising complementarity determining regions (CDRs) LCDR1, LCDR2, and LCDR3, wherein HCDR3 comprises the amino acid sequence of SEQ ID NO. 3, and wherein: (a) HCDR1 comprises the amino acid sequence of SEQ ID NO: 1, except that at least one amino acid residue of SEQ ID NO: 1 is replaced with a different amino acid residue; (b) HCDR2 comprises the amino acid sequence of SEQ ID NO: 2, except that at least one amino acid residue of SEQ ID NO: 2 is replaced with a different amino acid residue; (c) LCDR1 comprises the amino acid sequence of SEQ ID NO: 4, except that at least one amino acid residue of SEQ ID NO: 4 is replaced with a different amino acid residue; (d) LCDR2 comprises the amino acid sequence of SEQ ID NO: 5, except that at least one amino acid residue of SEQ ID NO: 5 is replaced with a different amino acid residue; (e) LCDR3 comprises the amino acid sequence of SEQ ID NO: 6, except that at least one amino acid residue of SEQ ID NO: 6 is replaced with a different amino acid residue; or (f) any combination of (a)-(e).

The disclosure also provides methods for treating or diagnosing a neurodegenerative disease in a human using the aforementioned monoclonal antibody, or a composition comprising the monoclonal antibody.

### BRIEF DESCRIPTION OF THE DRAWING(S)

FIG. 1 is a schematic diagram illustrating a method for systematically maturing the affinity and specificity of anti-Aβ amyloid antibodies. A lead single-chain antibody fragment (scFv) specific for Aβ fibrils was mutated by targeting solvent-exposed and naturally diverse sites in three complementarity-determining regions (CDRs), including heavy chain CDR2 (H2) and light chain CDRs 1 (L1) and 3 (L3). The library was displayed on yeast and sorted negatively against disaggregated Aβ and positively against aggregated Aβ using magnetic-activated cell sorting. The enriched libraries were subjected to deep sequencing and identified clones with mutations predicted to be favorable were evaluated in terms of their affinity and conformational specificity.

FIGS. 2A and 2B are schematic diagrams illustrating the design of an AF1 antibody library for affinity maturation that targets naturally diverse and solvent-exposed CDR sites with mutations that are common in human antibodies. FIG. 2A shows sites in heavy (H2) and light (L1 and L3) chain CDRs (HCDR2—SEQ ID NO: 73; LCDR1—SEQ ID NO: 72) identified based on their solvent exposure, diversity in human antibodies, and compatibility with sets of mutations most commonly observed in human antibodies. The wild-type residues at each site (boxed in red) were included in the library, and the average frequency (%) of each residue observed at each site in human antibodies was color coded. Some of the most common residues in human antibodies were not sampled because they are incompatible with degenerate codons encoding the wild-type residue and other favorable residues. FIG. 2B shows a summary of the designed antibody library at eleven CDR sites in HCRD2 (SEQ ID NO: 73), LDCR1 (SEQ ID NO: 72), and LCDR3 that includes the wild-type residue and three to five mutations that aim to sample combinations of residues most commonly observed in human antibodies.

FIGS. 3A and 3B are graphs summarizing the results of sorting yeast-displayed antibody library against Aβ fibrils. FIG. 3A shows the percentage of retained cells for both fibrillar and disaggregated Aβ after the library was subjected to five rounds of selection against Aβ fibrils. FIG. 3B shows the ratio of antibody-displaying yeast cells bound to fibrillar relative to disaggregated Aβ in each round of selection. 10⁷ antibody-displaying yeast cells were used in each round of selection.

FIGS. 4A-4E show results from deep sequencing to identify sets of affinity-enhancing mutations. Antibody libraries were sequenced before and after sorts 4 and 5 against Aβ fibrils, and sets of six mutations were identified that were strongly correlated with increased enrichment relative to wild-type. FIGS. 4A and B are graphs illustrating correlation between the frequency of clones with a particular set of six mutations (T53A and Y56N in HCDR1, D28N, N30A and T31Y in LCDR1, and T94Y in LCDR3) and enrichment ratios for clones observed with the corresponding mutations after round 4 (FIG. 4A) and round 5 (FIG. 4B) of selection. In FIGS. 4A and 4B, the lines (logistic regression curves) are guides to the eye. FIG. 4C is schematic diagram illustrating that antibody variants with sets of six mutations display strong correlation with improved enrichment for recognizing Aβ fibrils relative to wild-type (AF1) In FIGS. 4A-4C, the Spearman correlation coefficients were evaluated using antibody variants with wild-type or mutant residues at the specified positions regardless of their residues at the other five mutated positions (HCDR2—SEQ ID NO: 73; LCDR1—SEQ ID NO: 72). Enrichment ratios were calculated as the ratios of the frequencies of each variant observed in the sequencing results for the fibril selections (output) divided by the corresponding values for the input frequencies. FIGS. 4D and 4E are plots showing a comparison of Spearman correlation coefficients for sets of mutations identified in multiple rounds of antibody library sorting against Aβ fibrils. Antibody libraries were sequenced and sets of mutations were identified as described above. All possible combinations of five and six mutation sets were evaluated, and only those that were statistically significant (p-value<0.05) in both Rounds 4 and 5 were reported. The percentage of sets of mutations with Spearman correlation coefficients that were either both positive or both negative was 98.636% (5 mutation sets, FIG. 4D) and 99.313% (6 mutation sets, FIG. 4E). FIG. 4F is a schematic diagram showing antibody variants with sets of five mutations that display strong correlation with improved enrichment for recognizing Aβ fibrils relative to wild-type (AF1) (HCDR2—SEQ ID NO: 73; LCDR1—SEQ ID NO: 72). The mutation sets were identified as described above.

FIGS. 5A and 5B are graphs showing concentration-dependent binding of selected antibody variants to immobilized Aβ fibrils FIGS. 5C and 5D are graphs showing apparent affinity (EC₅₀) of selected antibody variants for Aβ fibrils. FIG. 5E is a graph illustrating competitive binding of antibodies (30 nM) pre-incubated with disaggregated Aβ in solution to immobilized fibrils. FIGS. 5F and 5G are graphs showing percentage of bound antibody to Aβ fibrils for antibodies (30 nM) pre-incubated with disaggregated Aβ (1000 nM). Control antibodies (aducanumab and crenezumab) were used for comparison. The results shown are average values and the error bars are standard deviations (two independent repeats). FIGS. 5H and 5I are images of SDS-PAGE analysis of the anti-Aβ antibodies evaluated in Examples. Antibodies were evaluated prior to boiling and reduction (+) or after boiling and reduction (+). The gels (10% Bis-Tris) were visualized using Coomassie blue staining.

FIG. 6A includes images of an immunoblot analysis of the conformational and sequence specificity of the selected Aβ antibodies. Disaggregated Aβ and fibrillar Aβ, IAPP, and αSyn were immobilized on nitrocellulose membranes and probed with anti-Aβ antibodies (10 nM in PBS with 1% milk). The blots were imaged after relatively short exposure times (30 seconds) except for AF1 (45 minute exposure). A loading control blot was detected using colloidal silver stain. The experiments were repeated three times and a representative example is shown. FIG. 6B includes images of immunoblot analysis of the conformational and sequence specificity of the selected Aβ antibodies using a long exposure time. The blots were processed and imaged as described in FIG. 6A except that all of the blots were processed after a relatively long exposure time (45 minutes). A loading control blot was detected using colloidal silver stain. The experiments were repeated three times and a representative example is shown.

FIG. 7 is an image of an immunoblot analysis of transgenic (5×FAD) and wild-type mouse brain samples using anti-Aβ antibodies. Brain samples obtained from 5×FAD and wild-type mice were immobilized on nitrocellulose membranes and probed with anti-Aβ antibodies (50 nM in PBS with 1% milk). The blots were imaged after a relatively short exposure (15 seconds). Ponceau S stain was used as loading control (LC). The experiments were repeated three times and a representative example is shown.

FIG. 8 includes images of Western blot analysis of 5×FAD and wild-type mouse brain samples using anti-Aβ antibodies. Brain samples isolated from 5×FAD and wild-type mice were processed (with or without boiling) via SDS-PAGE, transferred to nitrocellulose membranes, and probed with a subset of anti-Aβ antibodies (100 nM in PBS with 1% milk). The blots were imaged after 6 min of exposure Ponceau S stain was used as loading control. The experiments were repeated three times and a representative example is shown.

FIG. 9A is a graph showing antibody non-specific binding to soluble membrane proteins. The soluble membrane proteins were biotinylated and their binding to immobilized antibodies was evaluated via flow cytometry. FIG. 9B is a graph showing percentage of monomeric antibody evaluated via size-exclusion chromatography. FIG. 9C is a graph showing antibody melting temperature (midpoint of unfolding) evaluated using dynamic scanning fluorimetry. The values shown are averages and the error bars are standard deviations (three independent repeats). FIG. 9D is a graph showing non-specific binding analysis of anti-Aβ antibodies purified using both Protein A and size-exclusion chromatography. The non-specific binding results were performed as described above except that the antibodies were purified using both Protein A and size-exclusion chromatography. The values are averages and the error bars are standard deviations (three independent repeats). FIG. 9E is a graph showing analytical size exclusion chromatography analysis of anti-Aβ antibodies. Representative chromatograms for anti Aβ-antibodies after Protein A purification. The running buffer was PBS with 200 mM arginine (pH 7.4). FIG. 9F is a graph showing antibody thermal unfolding curves evaluated using differential scanning fluorimetry. Representative unfolding fluorescence curves are shown for antibodies obtained using the Thermal Shift dye.

FIG. 10A is a graph showing concentration-dependent binding of clone 97A3 to Aβ fibrils relative to its parental antibody (clone 97) and aducanumab. FIG. 10B is a graph showing a binding analysis of antibodies (30 nM) pre-incubated with different concentrations of disaggregated Aβ prior to binding to immobilized Aβ fibrils. FIG. 10C is a graph showing antibody non-specific binding to soluble membrane proteins. In FIGS. 10A and 1 B, the experiments were performed as described in FIG. 5. In FIG. 10C, the experiments were performed as described in FIG. 9.

FIG. 11A is a representative chromatogram for Aβ antibody 97A3 after Protein A purification. The running buffer was PBS with 200 mM arginine (pH 7.4). FIG. 11B is a representative thermal unfolding curve for clone 97A3 using a fluorescent thermal shift dye. The experiments were repeated three times and a representative example is shown.

FIG. 12 is a schematic overview of an exemplary approach for affinity maturing Aβ antibodies. A lead Aβ conformational antibody, in a single-chain variable fragment (scFv) format, was affinity matured by first generating a sub-library via site-specific (degenerate codon) mutagenesis at ten sites in two CDRs (light chain CDR2 and heavy chain CDR1). Next, the antibody library was displayed on the surface of yeast and sorted positively for binding to Aβ fibrils via magnetic-activated cell sorting and negatively for a lack of binding to disaggregated Aβ via fluorescence-activated cell sorting. The resulting enriched libraries were deep sequenced, and multiple methods were tested for predicting antibody mutants with large increases in affinity. The predicted antibodies were then cloned as IgGs and tested for their affinity and conformational specificity for Aβ aggregates using synthetic and biological samples.

FIGS. 13A and 13B are graphs of the selection of Aβ conformational antibodies. An antibody sub-library was designed with degenerate NNK codons at five sites in light chain CDR2 and five sites in heavy chain CDR1. The library was displayed on the surface of yeast as single-chain antibody fragments (scFvs) and subjected to seven rounds of positive sorting against Aβ fibrils via magnetic-activated cell sorting (sorts 1-3 and 5-8). The percentage of cells retained after each positive selection relative to the input (10⁹ cells for round 1 and 10⁷ cells for remaining rounds) are shown in FIG. 13A. FIG. 13B is a graph of sort 4, where the library was sorted negatively against disaggregated Aβ (1000 nM) by fluorescence-activated cell sorting (FACS) to eliminate antibodies with strong binding to Aβ monomer by collecting antibody-displaying cells in the R7 gate.

FIGS. 14A, 14B, and 14C show the deep sequencing analysis of enriched libraries for identifying Aβ antibodies with high affinity and conformational specificity. The enriched libraries were deep sequenced after rounds 2-3 and 5-7, and the sequencing data was used to create position-specific scoring matrices (PSSMs) from rounds 5-7. The differences between PSSMs from consecutive rounds [e.g., round 6 (R6) PSSM minus round 5 (R5) PSSM, ΔPSSM (R6-R5)] were computed (FIGS. 14A and 14B). This was done to create difference matrices for round 7 (R7) relative to R6 and R6 relative to R5. Large positive values (dark red) signified strong enrichment of a given residue at a specific CDR site, while large negative values signified a strong depletion of a given residue at a specific CDR site. The difference matrices were used to score individual antibody clones observed in rounds 2, 3, 5 and 6 clones by calculating the difference PSSM score at each of the ten mutated sites and then summing the scores over the ten sites for the R6-RS difference matrix (FIG. 14A) and R7-R6 difference matrix (FIG. 14B). For example, a representative antibody mutant is shown at the bottom of each matrix that is scored at each of the ten mutated positions in light chain CDR2 and heavy chain CDR1, and then the summed (total) scores for each matrix are reported. The difference matrix scores were used to identify the most promising clones that displayed high values for both difference matrices (FIG. 14C). Of the 244 mutants identified, which were observed in rounds 2, 3, 5 and 6, the clones with the top 40 scores from both matrices (marked with the dotted box) were chosen for further evaluation.

FIG. 15 is a graph of the relative affinities of the selected antibodies evaluated using flow cytometry analysis of soluble IgGs binding to Aβ fibrils immobilized on micron-sized magnetic beads (Dynabeads). Soluble IgGs (0.1-100 nM) were incubated with beads coated with Aβ fibrils (PBS with 1% milk) and detected using anti-Fc Alexa Fluor 647 antibodies. Two clinical-stage antibodies were included as controls, namely aducanumab and crenezumab. These IgGs contain the variable regions of the clinical-stage antibodies and the constant regions from a common IgG1 framework. Therefore, the reported clinical-stage antibodies contain sequence differences relative to the actual antibody drugs. The binding curves are averages from two independent experiments and the error bars are standard deviations.

FIG. 16 is a graph of the average APSSM scores, which are the averages of the ΔPSSM (R7-R6) and APSSM (R6-R5) scores, computed for each antibody and compared to their relative measured affinities in the IgG format. The relative affinity is plotted as the inverse value (1/EC₅₀) and the error bars represent the standard deviations (two independent experiments). The dashed line represents the relative affinity of the parental (wild type) antibody.

FIG. 17 is a graph of the conformational specificities of the Aβ IgGs evaluated using flow cytometry and fibril-coated magnetic beads. The IgGs (10 nM) were first incubated with disaggregated Aβ (0.1-1000 nM) followed by incubation with Aβ fibril-coated beads (PBS with 1 mg/mL BSA and 1% milk). Finally, the bound antibody was detected via flow cytometry using anti-Fc Alexa Fluor 647. The binding curves are averages from two independent experiments and the error bars are standard deviations.

FIG. 18 is immunodot blot analysis of Aβ antibody detection of brain homogenates from transgenic 5× FAD and wild-type mice. Brain homogenates from transgenic 5× FAD and wild type (control) mice were first immobilized on nitrocellulose membrane followed by incubation with IgGs at 50 nM (TBS with 0.1% Tween 20 and 1% milk) overnight at 4° C. The signals were detected using chemiluminescence. Ponceau stained blot is used as a loading control (LC). Two clinical-stage antibodies, aducanumab (Adu) and crenezumab (Cre), were included as controls. A sequence-specific antibody (NAB228), which recognizes both soluble and aggregated Aβ, was also included as a control. The experiments were performed three times and a representative example is shown.

FIG. 19 is western blot analysis of Aβ antibodies with transgenic 5× FAD mice. Brain homogenates from transgenic 5× FAD and wild type (control) mice were first run on SDS-PAGE followed by transfer onto nitrocellulose membranes. Next, the membranes were incubated with IgGs at 100 nM (97A7 and 97A34) or 1000× dilution (NAB228) in TBS with 0.1% Tween 20 and 1% milk overnight at 4° C. The signals were detected using chemiluminescence. Ponceau stained blot is used as a loading control (LC). A sequence-specific antibody (NAB228), which recognizes both soluble and aggregated Aβ, was also included as a control. The experiments were performed three times and a representative example is shown.

FIG. 20 is immunofluorescence staining of transgenic 5× FAD mice brain tissues with Aβ antibodies. Fixed brain tissues from 5× FAD transgenic and wild-type mice were stained with DAPI (blue), a panel of conformational antibodies (97A5, 97A7, 97A34 and aducanumab; purple) and an Aβ sequence-specific antibody (NAB228; green). The tissue samples were incubated with 97A5, 97A7, 97A34 and aducanumab at (10 nM) or with NAB228 at 200× dilution overnight at 4° C. The conformational antibodies were detected via anti-human Fc Alexa Fluor 647 and the Aβ sequence-specific antibody was detected via anti-mouse Fc Alexa Fluor 488. The images are 50 μm, and the inset images are 15 μm.

FIG. 21 is immunodot blot analysis of Aβ antibody detection of homogenates from Alzheimer's disease human brains. Homogenates from Alzheimer's disease (AD) and healthy (control) human brains were first immobilized on nitrocellulose membranes and then incubated with Aβ antibodies (97A7 and 97A34 at 100 nM, aducanumab (adu) at 10 nM, and NAB228 at 1000× dilution) in PBST with 1% milk overnight at 4° C. The signals were detected using chemiluminescence. The experiments were performed three times and a representative example is shown. Ponceau stained blot is used as a loading control (LC).

FIGS. 22A, 22B, and 22C show the biophysical analysis of Aβ conformational antibodies. FIG. 22A is a graph of non-specific binding of Aβ antibodies to biotinylated soluble membrane proteins from CHO cells was measured via flow cytometry using IgGs immobilized on magnetic beads. Clinical-stage antibody controls were included for high non-specific binding (emibetuzumab) and low non-specific binding (elotuzumab). These two control antibodies were used to normalize the levels of non-specific binding for the Aβ antibodies. FIG. 22B is a graph of the percentage of monomeric protein (area under the curve) evaluated by analytical size exclusion chromatography after one-step (Protein A) purification. FIG. 22C is a graph of antibody melting temperatures (midpoint of first unfolding transition) evaluated using dynamic scanning fluorimetry. The data are averages of three (FIG. 22A), two (FIG. 22B), and two (FIG. 22C) independent experiments, and the error bars are standard deviations.

FIG. 23 is a summary of antibody library design. To affinity mature the parental antibody (97 WT), five positions (X) in light chain CDR2 (SEQ ID NO: 5) and five positions in heavy chain CDR1 (SEQ ID NO: 1) were mutated using NNK codons.

FIG. 24 is position-specific scoring matrices (PSSMs) for the deep sequencing data from rounds 5-7. The enriched libraries were deep sequenced after 5-7 rounds, and the sequencing data was used to create position-specific scoring matrices (PSSMs). Large positive values (dark red) signified strong enrichment of a given residue at a specific CDR site, while large negative values signified a strong depletion of a given residue at a specific CDR site. FIG. 25 is SDS-PAGE analysis of the Aβ IgGs evaluated in this study. Purified IgGs were evaluated prior to heating and reduction (−) and after heating and reduction (+). The gels (10% Bis-Tris) were visualized using Coomassie blue staining.

FIG. 26 is graphs of alternative metrics for selecting Aβ conformational antibodies. Three alternative metrics were compared to the measured relative affinity (1/EC50): frequency of each clone in round 7 (top); global enrichment ratio (ER), as defined as log base 2 of the frequency of a clone in round 7 divided by its frequency in round 2 (middle); and local ER defined as log base 2 of the frequency of a clone in round 7 divided by its frequency in round 6 (bottom). The dotted line signifies the cutoff for the top forty antibody clones for R7 frequency (top), global enrichment ratio (middle), and local enrichment ratio (bottom).

### DETAILED DESCRIPTION

The present disclosure is predicated, at least in part, on the development of a systematic directed evolution procedure for generating high quality, affinity-matured conformational antibodies against Alzheimer's Aβ fibrils. The affinity-matured antibodies retain high conformational specificity for Aβ aggregates and display extremely low levels of nonspecific interactions. The methods described herein generate antibodies with unique combinations of desirable properties that improve the generation of high-quality conformational antibodies against diverse types of aggregated conformers.

### Definitions

To facilitate an understanding of the present technology, a number of terms and phrases are defined below. Additional definitions are set forth throughout the detailed description.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions The term “framework region,” as used herein, refers to the relatively conserved amino acid sequences within the variable region which are located between the CDRs. There are four framework regions in each variable domain, which are designated FR1, FR2, FR3, and FR4. The framework regions form the B sheets that provide the structural framework of the variable region (see, e.g., C. A. Janeway et al. (eds.), Immunobiology, 5th Ed., Garland Publishing, New York, N. Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

| As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10⁻² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof Examples of antibody fragments include, but are not limited to, (i) a Fab fragment, which is a monovalent fragment consisting of the V_{L}, V_{H}, C_{L}, and C_{H1} domains, (ii) a F(ab′)2 fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The term “library,” as used herein, refers to a plurality of polynucleotides, proteins, or cells comprising a collection of two, or two or more, non-identical but related members.

“Affinity maturation” is the process by which the immune system generates antibodies of higher affinities during a response to antigen. Affinity maturation is the result of iterative rounds of somatic hypermutation of immunoglobulin genes in B cells coupled with clonal selection for antigen binding. This iterative process occurs in germinal centers (GCs), which are structures within secondary lymphoid tissues, and proceeds for weeks after acute infection or vaccination, or for many cycles during chronic infection (MacLennan, I. C., Annu Rev Immunol., 12: 117-39 (1994)). The resulting antibodies can be highly mutated from their germline-encoded counterparts, with increases of several orders of magnitude in affinity for antigen compared to the corresponding naïve B cell receptors (BCRs) (Chan, T. D., Brink, R, Immunol Rev , 247(1): 11-23 (2012)). The term “somatic hypermutation (SHM),” as used herein, refers to the mutation of a polynucleotide sequence initiated by, or associated with the action of activation-induced cytidine deaminase (AID), uracil glycosylase, and/or error prone polymerases on that polynucleotide sequence. The binding affinities of the variable regions of immunoglobulins are altered by AID-promoted mutations during antigen-stimulated proliferation of B cells. These somatic hypermutations are transcribed and translated into thousands of slightly different immunoglobulins coded by the hypermutated V regions. The complementarity determining regions of these antibodies possess different affinities for the encountered antigen. The term “clonal selection,” as used herein, refers to the phenomenon whereby a previously unencountered cognate antigen (epitope) can stimulate naïve B lymphocytes to proliferate and differentiate into clones of memory B cells and plasma cells that produce antibodies with the highest affinity for the antigen. B cells with the highest affinity BCR against the encountered antigen will be selected for proliferation, antibody production, and commitment to an antigen-specific memory lineage.

The term “directed evolution,” as used herein, refers to a protein engineering method that mimics the process of natural selection to steer proteins or nucleic acids toward a user-defined goal. Directed evolution relies on an iterative two-step protocol, initially generating molecular diversity by random mutagenesis and in vitro recombination, followed by identifying library members with improvements in a desired phenotype by high-throughput screening or selection.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

### Amyloid Beta (Aβ) Fibrils

The disclosure provides a monoclonal antibody directed against fibrils of amyloid beta (Aβ) peptides, or an antigen-binding fragment thereof. Neurodegenerative diseases such as Alzheimer's disease (AD), Parkinson's disease (PD), Huntington's disease (HD), amyotrophic lateral sclerosis (ALS), and prion diseases have common cellular and molecular mechanisms, most notably protein aggregation and inclusion body formation (Ross, C. A., and Poirier, M. A. Nat. Med. 2004, 10, S10-S17 (2004)). Protein aggregates usually comprise fibers containing misfolded protein with a beta-sheet conformation, referred to in the art as “amyloid” (Soto, C. Nat. Rev. Neurosci. 4, 49-60 (2003)). The “amyloid hypothesis” (Haass C, Selkoe D J, Cell, 75: 1039-42 (1993); Glenner G. G., Wong C. W., Biochem Biophys Res Commun, 425: 534-539 (2012); and Selkoe, D. J., Hardy, J., EMBO Mol Med 2016; 8: 595-608 (2016)) proposes Aβ protein as the main cause of the disease and suggests that misfolding of the extracellular Aβ protein accumulated in senile plaques (Bloom, G. S. JAMA Neurol 2014; 71: 505-8) and the intracellular deposition of misfolded tau protein in neurofibrillary tangles cause memory loss and confusion and result in personality and cognitive decline over time. With respect to Alzheimer's disease, the two hallmark pathologies required for a diagnosis are the extracellular plaque deposits of the β-amyloid peptide (Aβ) and the flame-shaped neurofibrillary tangles of the microtubule binding protein tau.

The Aβ peptide is approximately 4 kDa and is derived from the larger β-amyloid precursor protein, APP. APP consists of a single membrane-spanning domain, a large extracellular glycosylated N-terminus and a shorter cytoplasmic C-terminus. APP has been implicated as a regulator of synaptic formation and repair, anterograde neuronal transport, and iron export. APP is cleaved by β-secretases and γ-secretases to produce a 37 to 49 amino acid residue Aβ peptide (Nunan J, Small D H, FEBS Lett 2000; 483: 6-10). Human APP can be processed via two alternative pathways: amyloidogenic and nonamyloidogenic. Amyloidogenic processing of APP involves sequential cleavages by β- and γ-secretase at the N and C termini of Aβ, respectively (Joshi G, Wang Y, BioEssays 2015; 37: 240-7). The 99-amino-acid C-terminal fragment of APP (C99) generated by β-secretase cleavage can be internalized and further processed by γ-secretase at multiple sites to produce cleavage fragments of 43, 45, 46, 48, 49 and 51 amino acids that are further cleaved to the main final Aβ forms, the 40-amino-acid Aβ40 and the 42-amino-acid Aβ42, in endocytic compartments (Olsson et al., J Biol Chem 2014; 289: 1540-50; and Takami et al., J Neurosci 2009; 29: 13042-52). Mutations in the human APP gene cause the development of amyloid plaques and Alzheimer's-like brain pathology, especially in early-onset familial Alzheimer's disease (EOFAD) (Games et al., Nature 1995; 373: 523-7; and Hsiao et al., Science 1996; 274: 99-102).

The process of amyloidogenic protein aggregation is complex (Chiti, F., and Dobson, C. M., Annu. Rev. Biochem. 75, 333-366 (2006)). Aβ monomers may aggregate into various types of assemblies, including oligomers, protofibrils, and amyloid fibrils. Amyloid fibrils are larger and insoluble, and they can further assemble into amyloid plaques, while amyloid oligomers are soluble and may spread throughout the brain. The primary amino acid sequence of Aβ was first discovered from extracellular deposits and amyloid plaques in 1984 (Glenner and Wong, supra). The primary amino acid sequence of the 42-amino-acid Aβ isoform Aβ42 is known (see, e.g., Chen et al., Acta Pharmacol Sin, 38, 1205-1235 (2017); doi: 10.1038/aps.2017.28). Amyloid plaques with Aβ as the main component are most commonly found in the neocortex in the brain of Alzheimer's disease patients.

Aβ peptide can rapidly aggregate to form fibrils that deposit into amyloid plaques, and many pathways may lead to Aβ peptide aggregation (Chen et al., supra). The 3D structure of residues 15-42 of Aβ42 has been shown to adopt a double-horseshoe-like cross-β-sheet entity with maximally buried hydrophobic side chains, in which residues 1-14 are partially ordered and in a β-strand conformation, which is the more neurotoxic species, aggregates much faster, and dominates in senile plaques in Alzheimer's disease patients (Chen et al., supra). While amyloid fibrils are larger, insoluble, and assemble into amyloid plaques forming histological lesions that are characteristic of Alzheimer's disease, Aβ oligomers are soluble and may spread throughout the brain. The relationship between oligomers and fibrils has not yet been elucidated. Aβ oligomers and fibrils share similar structural elements, as they both appear to be extended or beta sheet structures and both display similar amounts of main chain hydrogen bonding that is resistant to exchange. Amyloid oligomers and fibrils also appear to contain mutually exclusive and non-overlapping conformations recognized as generic antibody epitopes that are common to amyloids of different sequences (Oddo et al., Neuron 2003; 39: 409-21; and Blackley et al., J Mol Biol 2000; 298: 833-40).

Aβ forms a myriad of structures in the monomeric and oligomeric states, all of which result in similar fibril structures. Amyloid fibrils of Aβ form a parallel, in-register cross β-sheet structure. The accumulation of Aβ into long, unbranched fibrils is a hallmark of the disease, as is the loss of neurons due to cell death in parallel with the Aβ aggregation process. The different Aβ forms, however, may contribute to neurodegeneration at different stages of the disease. For example, accumulating Aβ first forms Aβ oligomers and gradually deposits as fibrils and senile plaques. In addition, tau protein becomes hyperphosphorylated in response to kinase/phosphatase activity changes mediated by Aβ aggregation, leading to the formation of neurofibrillary tangles (NFTs), neuronal and eventual synaptic dysfunction, and finally Alzheimer's disease. Furthermore, the aggregation of Aβ may also produce free radicals as reactive oxygen species (ROS) that react rapidly with proteins or lipids, resulting in the formation of “toxic” oxidized proteins and peroxided lipids. Sustained elevation of Aβ levels and continuous aggregation might also promote a chronic response of the innate immune system by activating microglia, which can lead to neuronal loss through direct phagocytosis. The structure, function, and neurotoxicity of Aβ peptides is further described in, e.g., Chen et al., supra.

### Anti-Aβ Monoclonal Antibodies

In some embodiments, the monoclonal antibody specifically recognizes a conformational epitope of Aβ fibrils. The term “conformational epitope,” as used herein, refers to an antigenic protein composed of amino acid residues that are spatially near each other on the antigen's surface and are brought together by protein folding. In contrast, a “linear epitope” (also referred to as a “sequential epitope”) comprises a sequence of continuous amino acids that is sufficient for antibody binding. In the context of the present disclosure, the monoclonal antibody specifically recognizes and binds to different conformations (referred to as “conformers”) of amyloid-forming proteins. Conformational antibodies specific for different conformers of amyloid-forming proteins are important for detecting, disrupting, and reversing toxic protein aggregation. The generation of conformational antibodies directed against amyloid-forming proteins has been attempted (Hatami et al., J Biol Chem, 289: 32131-32143 (2014); Kayed et al., Science, 300: 486-489 (2003); Julian et al., J Biol Chem, 294: 8438-8451 (2019); Stimple et al., Biotechnol Bioeng, 116: 1868-1877 (2019); Lee et al., J Biol Chem, 291: 2858-2873 (2016)); however, several problems are associated with generating such conformational antibodies. First, the nature of amyloidogenic antigens is extremely complex and particularly unattractive for typical antibody selection methods due to their insolubility, heterogeneity in terms of size and conformation, hydrophobicity, and multivalency. Second, the use of immunization to generate such antibodies is limited due to uncontrolled presentation of aggregated antigens to the immune system and immunodominant epitopes. Third, the use of conventional directed evolution methods such as yeast surface display is limited by the inability to use fluorescence-activated cell sorting due to the lack of soluble antigens. These and other challenges typically result in antibodies that recognize protein aggregates with either conformational specificity (e.g., common fibril structure) or sequence specificity (e.g., linear peptide epitopes) but not both. The present disclosure overcomes these problems by providing high quality, affinity-matured monoclonal antibodies directed against conformational epitopes and sequence-specific epitopes of Aβ fibrils.

In this regard, directed evolution methods have previously been used for discovering lead antibodies with high conformational specificity (Julian et al., Biol Chem, 294, 8438-8451 (2019)). This approach involved designing single-chain (scFv) antibody libraries with focused mutagenesis in the antibody CDR (e.g., the heavy chain CDR3) that is most commonly involved in antigen binding. Combinations of mutations were sampled that are most commonly observed in natural antibodies based on tens of thousands of human antibody CDRs (Tiller et al., Front Immunol 8, 986 (2017)). From such libraries, a lead antibody was identified, referred to as “AF1,” that recognizes amyloid fibrils of the Aβ42 peptide with high conformational and sequence specificity (Julian et al., Biol Chem 294, 8438-8451 (2019)). This antibody displays much weaker affinity for soluble Aβ42 and extremely low levels of non-specific binding even at high antibody concentrations (100 nM). However, the specificity of AF1 is similar to several clinical-stage antibodies with high specificity.

Described herein is a method for affinity maturation of the AFI monoclonal against Aβ fibrils to increase affinity while maintaining strict conformational and sequence specificity as well as low levels of non-specific binding. The methods described herein address several challenges associated with generating conformational antibodies against Aβ fibrils. For example, most mutations that increase the affinity of such conformational antibodies also increase specific interactions with soluble Aβ (reduced conformational specificity) or non-specific interactions (reduced sequence specificity), or both. In addition, the multivalent nature of protein aggregates frustrates the selection of affinity-enhancing mutations due to avidity effects. Another challenge is the selection of sites to mutate as well as sets of mutations to sample in order to maximize the likelihood of obtaining matured antibody variants with high specificity and low levels of non-specific interactions. The present disclosure provides an integrated approach for affinity-maturing conformational antibodies specific for Aβ fibrils, which results in antibody variants with favorable combinations of binding properties relative to Aβ clinical-stage antibodies.

An exemplary monoclonal antibody of the present disclosure comprises a heavy chain variable region (VH) comprising complementarity determining regions (CDRs) HCDR1, HCDR2, and HCDR3 and a light chain variable region (VL) comprising complementarity determining regions (CDRs) LCDR1, LCDR2, and LCDR3, wherein HCDR3 comprises the amino acid sequence of SEQ ID NO: 3, and wherein: (a) HCDR1 comprises the amino acid sequence of SEQ ID NO: 1, except that at least one amino acid residue of SEQ ID NO: 1 is replaced with a different amino acid residue; (b) HCDR2 comprises the amino acid sequence of SEQ ID NO: 2, except that at least one amino acid residue of SEQ ID NO: 2 is replaced with a different amino acid residue; (c) LCDR1 comprises the amino acid sequence of SEQ ID NO: 4, except that at least one amino acid residue of SEQ ID NO: 4 is replaced with a different amino acid residue; (d) LCDR2 comprises the amino acid sequence of SEQ ID NO: 5, except that at least one amino acid residue of SEQ ID NO: 5 is replaced with a different amino acid residue; (e) LCDR3 comprises the amino acid sequence of SEQ ID NO: 6, except that at least one amino acid residue of SEQ ID NO: 6 is replaced with a different amino acid residue; or (f) any combination of (a)-(e).

In some embodiments, the HCDR1 comprises the amino acid sequence

(SEQ ID NO: 54)
SGYNIKATYIH,
(SEQ ID NO: 55)
SGFNIKGTYSH,
or
(SEQ ID NO: 56)
SGYNIKDTYIH

In some embodiments, the HCDR2 comprises the amino acid sequence

(SEQ ID NO: 28)
RIYPNSGATRYAGSVKG,
(SEQ ID NO: 29)
RIYPASGTTRYAGSVKG,
(SEQ ID NO: 30)
RIYPASGTTRYAASVKG,
(SEQ ID NO: 31)
RIYPSNGYTRYAGSVKG,
(SEQ ID NO: 32)
RIYPASGSTRYAASVKG,
(SEQ ID NO: 33)
RIYPASGATRYADSVKG,
(SEQ ID NO: 34)
RIYPSNGYTRYAASVKG,
(SEQ ID NO: 35)
RIYPASGSTRYAGSVKG,
or
(SEQ ID NO: 36)
RIYPASGYTRYAASVKG.

In other embodiments, the LCDR1 comprises the amino acid sequence

(SEQ ID NO: 37)
RASQNVYAAVT,
(SEQ ID NO: 38)
RASQNVYSAVS,
(SEQ ID NO: 39)
RASQNVYNAVT,
(SEQ ID NO: 40)
RASQNVYSAVT,
(SEQ ID NO: 41)
RASQNVAYAVT,
(SEQ ID NO: 42)
RASQNVAYAVS,
or
(SEQ ID NO: 43)
RASQNVASAVT.

In some embodiments, the LCDR3 comprises the amino acid sequence QQHSTYPPT (SEQ ID NO: 44), QQHYTYPPT (SEQ ID NO: 45), QQHNTYPPT (SEQ ID NO: 46), or QQHSTSPPT (SEQ ID NO: 47).

The monoclonal antibody may comprise one or any combination of an HCDR2 of SEQ ID NOs: 28-36, an LCDR1 of SEQ ID NOs: 37-43, and/or an LCDR3 of SEQ ID NOs: 44-47. The monoclonal antibody may comprise one or any combination of an HCDR1 of SEQ ID NOs: 54-56, an HCDR2 of SEQ ID NOs: 28-36, an LCDR1 of SEQ ID NOs: 37-43, and/or an LCDR3 of SEQ ID NOs: 44-47.

In some embodiments, the monoclonal antibody comprises a heavy chain variable region and a light chain variable region. Exemplary pairs of heavy chain and light chain variable region amino acid sequences are set forth below in Table 1 and are deposited in the GenBank database under Accession Nos. MT635022, MT635023, MT635024, MT635025, MT635026, MT635027, MT635028, MT635029, MT635030, MT635031, and MW202274.

TABLE 1
Exemplary Heavy and Light Chain Variable
Region Amino Acid Sequences

| VH Amino Acid | VL Amino Acid | |
|---|---|---|
| SEQ ID NO: | SEQ ID NO: | Clone # |
| 7 | 8 | 88 |
| 9 | 10 | 89 |
| 11 | 12 | 91 |
| 13 | 14 | 92 |
| 15 | 16 | 93 |
| 17 | 18 | 95 |
| 19 | 20 | 97 |
| 21 | 22 | 98 |
| 23 | 24 | 101 |
| 25 | 26 | 102 |
| 19 | 27 | 97A3 |
| 49 | 51 | 97A5 |
| 19 | 48 | 97A6 |
| 49 | 50 | 97A7 |
| 53 | 20 | 97A32 |
| 52 | 20 | 97A34 |
| 52 | 20 | 97A35 |

In other embodiments, the heavy chain variable region amino acid sequence is at least 90% identical (e.g., at least 91%, at least 92%, at least 93%, at least 94%, at least 95%, at least 96%, at least 97%, at least 98%, at least 99%, or 100% identical) to any of SEQ ID NOs: 7, 9, 11, 13, 15, 17, 19, 21, 23, or 25 and the light chain variable region amino acid sequence is at least 90% identical (e.g., at least 91%, at least 92%, at least 93%, at least 94%, at least 95%, at least 96%, at least 97%, at least 98%, at least 99%, or 100% identical) to any of SEQ ID NOs: 8, 10, 12, 14, 16, 18, 20, 22, 24, or 26. In some embodiments, the heavy chain variable region amino acid sequence is SEQ ID NOs: 49, 52, or 53. In some embodiments, the light chain variable region amino acid sequence is SEQ ID NOs: 48,50, or 51.

In other embodiments, the heavy chain amino acid sequence is at least 90% identical (e.g., at least 91%, at least 92%, at least 93%, at least 94%, at least 95%, at least 96%, at least 97%, at least 98%, at least 99%, or 100% identical) to any of SEQ ID NOs: 57, 59, 61, 63, 65, 67, or 71 and the light chain amino acid sequence is at least 90% identical (e.g., at least 91%, at least 92%, at least 93%, at least 94%, at least 95%, at least 96%, at least 97%, at least 98%, at least 99%, or 100% identical) to any of SEQ ID NOs: 58, 60, 62, 64, 66, 68, or 72.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

One or more amino acids of the aforementioned monoclonal antibody or antigen fragment thereof can be replaced or substituted with a different amino acid. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg)

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free -OH can be maintained, and glutamine for asparagine such that a free -NH2 can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the monoclonal antibody or antigen-binding fragment thereof (e.g., insertion into the heavy and/or light chain variable region amino acid sequence). Any number of any suitable amino acids can be inserted into the amino acid sequence of the antibody or antigen-binding fragment thereof. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the antibody or antigen-binding fragment thereof. For example, 1-10 amino acids (e.g., 1, 2, 3, 4, 5, 6, 7, 8, 9, or 10 amino acids) may be inserted into the amino acid sequence of the monoclonal antibody or antigen-binding fragment thereof. In this respect, the amino acid(s) can be inserted into antibody or antigen-binding fragment thereof in any suitable location. Preferably, the amino acid(s) are inserted into a CDR (e.g., CDR1, CDR2, or CDR3) of the antibody or antigen-binding fragment thereof.

The amino acid sequences of inventive monoclonal antibody or antigen-binding fragment thereof is not limited to the specific amino acid sequences described herein. Indeed, the antibody or antigen-binding fragment thereof can comprise any heavy chain polypeptide or light chain polypeptide that competes with the inventive monoclonal antibody or antigen-binding fragment thereof for conformational binding to an Aβ peptide. Antibody competition can be assayed using routine peptide competition assays such as, for example, ELISA, Western blot, or immunohistochemistry methods (see, e.g., U.S. Pat. Nos. 4,828,981 and 8,568,992; and Braitbard et al., Proteome Sci., 4: 12 (2006)).

The monoclonal antibody may be a whole antibody, or an antigen-binding fragment of a whole antibody. As defined herein, antigen-binding antibody fragments encompassed by the present disclosure include, but are not limited to, F(ab′)₂, Fab′, Fab, Fv, scFv, dsFv, dAb, and single chain binding polypeptides. Antibody fragments and their therapeutic utility are further described in, e.g., Nelson, A. L., MAbs. 2010 Jan-Feb; 2(1): 77-83, Joosten et al., Microbial Cell Factories volume 2, Article number: 1 (2003); and Bates A, Power C A., Antibodies (Basel). 2019; 8(2): 28; doi: 10.3390/antib8020028). In some embodiments, the antigen-binding fragment is a single-chain variable fragment (scFv), which is an engineered antibody generated by the fusion of the heavy (VH) and light chains (VL) of immunoglobulins through a short polypeptide linker. Single chain variable domain (Fv) fragments (scFv) are used in the art in a variety of clinical and therapeutic applications, primarily due to their improved pharmacokinetic properties as compared to the parent monoclonal antibodies and the relative ease of producing them in large quantities at low cost (Monnier et al., Antibodies 2013, 2(2), 193-208; doi.org/10.3390/antib2020193; Safdari et al., Mol Med. 2016; 22: 258-270; and Lu, R., Hwang, Y., Liu, I. et al. Development of therapeutic antibodies for the treatment of diseases. J Biomed Sci 27, 1 (2020). doi.org/10.1186/s12929-019-0592-z).

In other embodiments, the monoclonal antibody is a whole antibody. As defined herein, a whole antibody comprises two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) The heavy chain C-terminal constant region contains the fragment crystallizable (Fc) domain, which determines antibody class and is responsible for humoral and cellular effector functions. Antibodies are divided into five major classes (or “isotypes”), IgG, IgM, IgA, IgD and IgE, which differ in their function in the immune system. IgGs are the most abundant immunoglobulins in the blood, representing 60% of total serum antibodies in humans. IgG antibodies may be subclassified as IgG1, IgG2, IgG3, and IgG4, named in order of their abundance in serum (IgG1 being the most abundant) (Vidarsson et al., Frontiers in Immunology. 5: 520 (2014)). A whole monoclonal antibody described herein may be of any suitable class and/or subclass. In some embodiments, the monoclonal antibody is of class IgG (e.g., IgG1, IgG2, IgG3, or IgG4). For example, the monoclonal antibody may be an IgG1 antibody.

As discussed above, the Fc domain mediates several effector functions of antibodies, such as binding to receptors on target cells and complement fixation (triggering effector functions that eliminate the antigen). In some embodiments, the Fc domain may be modified or engineered to alter its effector functions. For example, Fc domains may be modified to improve antibody-dependent cellular cytotoxicity (ADCC) and antibody-dependent cellular phagocytosis (ADCP), and to control serum half-life. In some embodiments, the Fc domain of the monoclonal antibody may be engineered to modulate affinity for an Fc receptor, such as Fcγ receptors (FcγRs) and the neonatal Fc receptor (FcRn). Indeed, optimization of the interactions between antibodies and FcγRs has emerged as a promising approach for enhancing the activity of therapeutic antibodies for the treatment of both cancer and autoimmune disease (Mimoto et al., Curr. Pharm. Biotechnol. 17, 1298-1314 (2016); Lazar et al., Proc. Natl Acad. Sci. USA 103, 4005-4010 (2006); Richards et al., Mol. Cancer Ther. 7, 2517-2527 (2008); Nordstrom et al., Breast Cancer Res. 13, R123 (2011); and Kang, T. H., Jung, S. T., Exp Mol Med 51, 1-9 (2019)). The Fc domain also may be modified to improve serum half-life, e.g., by engineering IgG Fc for higher FcRn binding (Zalevsky et al., Nat. Biotechnol. 28, 157-159 (2010); and Dall' Acqua et al., J Immunol. 169, 5171-5180 (2002)). In other embodiments, the Fc domain may be modified to create monovalency or antibody bispecificity for improving therapeutic potency. For example, an Fc domain may be generated that does not form a homodimer but remains as a soluble monomer, mFc, that exhibits high affinity for FcγRI but no detectable binding to FcγRIIIa. In other embodiments, a heterodimeric Fc domain may be generated to obtain bispecific properties for antigen binding to circumvent homodimer formation. Engineered Fc domains may be generated by inducing point mutations or by modifying glycosylation of the Fc domain (Saunders, K. O., Front Immunol. 2019; 10.1296; Kelley, R. F., Meng, Y. G., Liu et al., J Biol Chem. 2014;289:3571-90; Monnet et al., MAbs. 2014; 6: 422-36; Li et al., Proc Natl Acad Sci U S A. 2017; 114: 3485-90; and Lin et al., Proc Natl Acad Sci U S A. 2015; 112: 10611-6; Kang and Jung, supra).

The disclosure further provides a nucleic acid sequence encoding the aforementioned monoclonal antibody or antigen-binding fragment thereof. In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N. Y. (2012), and Ausubel et al., Current Protocols in Molecular Biology, Greene Publishing Associates and John Wiley & Sons, New York, N. Y. (1994)).

In addition to the nucleic acid encoding the monoclonal antibody or antigen-binding fragment thereof, the vector desirably comprises expression control sequences, such as promoters, enhancers, polyadenylation signals, transcription terminators, internal ribosome entry sites (IRES), and the like, that provide for the expression of the antibody-encoding nucleic sequence in a host cell. Exemplary expression control sequences are known in the art and described in, for example, Goeddel, Gene Expression Technology: Methods in Enzymology, Vol. 185, Academic Press, San Diego, Calif. (1990).

A vector comprising a nucleic acid sequence encoding the monoclonal antibody or antigen-binding fragment thereof may be introduced into a host cell that is capable of expressing the polypeptides encoded thereby, including any suitable prokaryotic or eukaryotic cell. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DHSα, DH10, MC1061 (ATCC No. 53338), and CC102). Suitable eukaryotic cells are known in the art and include, for example, yeast cells, insect cells, and mammalian cells. Examples of suitable yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of such cells are well known in the art (see, e.g., Ausubel et al., eds., Short Protocols in Molecular Biology, 5th ed., John Wiley & Sons, Inc., Hoboken, N.J. (2002)). Preferably, the mammalian cell is a human cell.

The disclosure also provides a composition comprising the monoclonal antibody or antigen-binding fragment thereof described herein. The composition desirably is a pharmaceutically acceptable (e.g., physiologically acceptable) composition, which comprises a carrier, preferably a pharmaceutically acceptable (e.g., physiologically acceptable) carrier, and the monoclonal antibody or antigen-binding fragment thereof. Any suitable carrier can be used within the context of the disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

The disclosure further provides a method of treating a neurodegenerative disease in a human. The method comprises administering an effective amount of the above-described monoclonal antibody or antigen-binding fragment thereof, or a composition comprising same, to a human having a neurodegenerative disease, whereupon the neurodegenerative disease is treated in the human. The disclosure also is directed to the use of the above-described monoclonal antibody in a method of treating a human with a neurodegenerative disease. The term “neurodegenerative disease,” as used herein, refers to a heterogeneous group of disorders that are characterized by the progressive degeneration of the structure and function of the central nervous system or peripheral nervous system. Examples of neurodegenerative diseases include, but are not limited to, Alzheimer's disease, Huntington's disease, Parkinson's disease, and dementia (e.g., Alzheimer's disease-related dementias (ADRDs)). Alzheimer's disease and Parkinson's disease are the most common neurodegenerative diseases. In 2016, an estimated 5.4 million Americans were living with Alzheimer's disease. An estimated 930,000 people in the United States could be living with Parkinson's disease by 2020. Thus, in some embodiments, the disclosed method is used to treat Alzheimer's disease or Parkinson's disease. The disclosed method, however, is not limited to the treatment of these specific neurodegenerative diseases.

Alzheimer's disease (AD) is an age-related, non-reversible brain disorder that develops over a period of years. Initially, patients experience memory loss and confusion, which may be mistaken for changes that are sometimes associated with normal aging. The symptoms of AD gradually lead to behavior and personality changes, a decline in cognitive abilities such as decision-making and language skills, and problems recognizing family and friends. AD ultimately leads to a severe loss of mental function. These losses are related to the worsening breakdown of the connections between certain neurons in the brain and their eventual death. AD is one of a group of disorders called dementias that are characterized by cognitive and behavioral problems. It is the most common cause of dementia among people aged 65 and older (National Institute of Neurological Disorders and Stroke). Currently, there are no known treatments to prevent AD onset or progression; however, four drugs have been approved by the FDA to treat AD symptoms. Donepezil (ARICEPT®), rivastigmine (EXELON®), and galantamine (RAZADYNE®) can treat mild to moderate AD symptoms. Donepezil was recently approved to treat severe AD as well. Memantine (NAMENDA®) has been approved to treat moderate to severe AD symptoms.

Parkinson's disease (PD) is a motor system disorder which causes unintended or uncontrollable movements of the body. The precise cause of PD is unknown, but some cases are hereditary while others are thought to occur from a combination of genetics and environmental factors that trigger the disease. In PD, brain cells become damaged or die in the part of the brain that produces dopamine, a chemical needed to produce smooth, purposeful movement. The four primary symptoms of PD are tremors, muscle rigidity, bradykinesia (e.g., slowing of spontaneous and automatic movement that can make it difficult to perform simple tasks or rapidly perform routine movements), and postural instability (e.g., impaired balance and changes in posture that can increase the risk of falls. Other PD symptoms may include difficulty swallowing, chewing, or speaking; emotional changes; urinary problems or constipation; dementia or other cognitive problems; fatigue; and problems sleeping. PD usually affects people around the age of 70 years but can occur earlier. PD affects more women than men (National Institute of Neurological Disorders and Stroke (ninds.nih.gov)). At present, there is no cure for PD, but a variety of medications can provide dramatic relief from symptoms. Affected individuals typically are given levodopa combined with carbidopa. Carbidopa delays the conversion of levodopa into dopamine until it reaches the brain. Anticholinergic drugs may help control tremor and rigidity. Other drugs, such as pramipexole, apomorphine, and ropinirole, mimic the role of dopamine in the brain, causing the nerve cells to react as they would to dopamine. The antiviral drug amantadine appears to reduce symptoms and may also be administered. Other drugs to treat PD include COMT inhibitors, which prolong the effects of levodopa by preventing the breakdown of dopamine, and MAO-B inhibitors, which block or reduce activity of the MAO-B enzyme that breaks down dopamine in the brain. In some cases, surgery may be appropriate for patients that to not respond to drugs. For example, deep brain stimulation (DBS) can be used to painlessly stimulate the brain to block signals that cause many of the motor symptoms of PD.

As used herein, the terms “treatment,” “treating,” and the like refer to obtaining a desired pharmacologic and/or physiologic effect. Preferably, the effect is therapeutic, e.g., the effect partially or completely cures a disease and/or adverse symptom attributable to the disease. To this end, the inventive method comprises administering a “therapeutically effective amount” of the monoclonal antibody, or composition comprising same. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the monoclonal antibody to elicit a desired response in the individual. For example, a therapeutically effective amount of a monoclonal antibody of the invention is an amount which reduces or eliminates Aβ fibrils in the brain

Alternatively, the pharmacologic and/or physiologic effect may be prophylactic, e.g., the effect completely or partially prevents a disease or symptom thereof. In this respect, the inventive method comprises administering a “prophylactically effective amount” of the monoclonal antibody or composition comprising same. A “prophylactically effective amount” refers to an amount effective, at dosages and for periods of time necessary, to achieve a desired prophylactic result (e.g., prevention of disease onset).

A typical dose can be, for example, in the range of 1 pg/kg to 20 mg/kg of animal body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The composition comprising the monoclonal antibody, or antigen-binding fragment thereof can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a human), the biological activity of the monoclonal antibody, or antigen-binding fragment thereof can be measured by any suitable method known in the art. For example, the biological activity can be assessed by determining the stability of the monoclonal antibody. The biological activity of the monoclonal antibody also can be assessed by determining its binding affinity to Aβ peptide fibrils. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 1 millimolar (mM) (e.g., from about 1 picomolar (pM) to about 1 nanomolar (nM), or from about 1 nM to about 1 micromolar (μM)). In some embodiments, the affinity of the inventive monoclonal antibody may be from about 1 nm to about 20 nm, and desirably from about 5 nm to about 10 nm. Antibody affinity for an antigen or epitope of interest can be measured using any art-recognized assay. Such methods include, for example, fluorescence activated cell sorting (FACS), separable beads (e.g., magnetic beads), antigen panning, and/or ELISA (see, e.g., Janeway et al. (eds.), Immunobiology, 5th ed., Garland Publishing, New York, N.Y., 2001).

In some embodiments, the monoclonal antibody, or composition comprising same, may be administered alone or in combination with other drugs. For example, the monoclonal antibody can be administered in combination with other agents for the treatment or prevention of Alzheimer's disease or Parkinson's disease disclosed herein.

In addition to therapeutic uses, the monoclonal antibody or antigen-binding fragment described herein can be used in diagnostic or research applications. In this respect, the disclosure provides a method of diagnosing a neurodegenerative disease in a human, which comprises contacting a sample obtained from a human suspected of having a neurodegenerative disease with the monoclonal antibody described herein, wherein binding of the monoclonal antibody to fibrils of Aβ peptides present in the sample indicates that the human has a neurodegenerative disease. In a similar manner, the monoclonal antibody or antigen-binding fragment thereof can be used in an assay to monitor Aβ fibril formation in a subject being tested for a neurodegenerative disease. Research applications include, for example, methods that utilize the monoclonal antibody and a label to detect Aβ fibrils in a sample, e.g., in a human body fluid or in a cell or tissue extract. The monoclonal antibody or antigen-binding fragment thereof may be employed in any suitable assay for measuring Aβ peptide fibrils or aggregates in a sample. Such assays include, but are not limited to, sandwich immunoassays, enzyme immunoassays (EIA), enzyme-linked immunosorbent assays (ELISA), lateral flow assays, competitive inhibition immunoassays (e.g., forward and reverse), competitive binding assays, Forster resonance energy transfer (FRET), one-step antibody detection assays, single molecule detection assays, radioimmunoassays (RIA), and FACS. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; and Adamczyk et al., Anal. Chim. Acta, 579(1): 61-67 (2006).

The monoclonal antibody or antigen-binding fragment thereof can be provided in a kit, e.g., a packaged combination of reagents in predetermined amounts with instructions for performing an assay using the antibody (e.g., an assay that detects Aβ fibrils). As such, the disclosure provides a kit comprising the antibody or antigen-binding fragment described herein and instructions for use thereof. The instructions can be in paper form or computer-readable form, such as a disk, CD, DVD, etc. Alternatively or additionally, the kit can comprise a calibrator or control, and/or at least one container (e.g., tube, microtiter plates, or strips) for conducting an assay, and/or a buffer, such as an assay buffer or a wash buffer. Ideally, the kit comprises all components, i.e., reagents, standards, buffers, diluents, etc., which are necessary to perform the assay. Other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following examples further illustrate the invention but, of course, should not be construed as in any way limiting its scope.

### EXAMPLES

The following materials and methods were used in the experiments described below.

### Aβ Solubilization and Fibril Preparation

Aβ fibrils were prepared as described previously (23). Lyophilized Aβ1-42 (Anaspec, AS20276) and biotinylated Aβ1-42 (Anaspec, AS23526-05) peptides were dissolved in hexafluoro-2-isopropanol (HFIP), aliquoted, and stored at −80° C. at 1 mg/mL and 0.17 mg/mL, respectively. For fibril preparation, aliquots were thawed and HFIP was evaporated overnight. Peptides were dissolved in 50 mM NaOH and ultracentrifuged at 221000× g at 4° C. for 1 hour. The supernatant (typically 45 μL) was collected, transferred to a new tube, and neutralized with nine times the volume (typically 405 μL) of acidified PBS (PBS with 4.7 mM HCl). The peptide concentration was determined by measuring the absorbance at 280 nm.

Unlabeled fibrils were assembled at 37° C .for at least 3 days without agitation by further diluting the soluble peptide in PBS to a final concentration of 12.5 μM along with the addition of 10% fibril seeds (1.25 μM of preformed fibrils). Biotinylated fibrils were assembled in a similar manner except the assemblies were doped with 1 or 10% biotinylated Aβ monomer (final concentration of Aβ monomer was 12.5 μM). After at least 3 days, the assemblies were ultracentrifuged at 221000× g for 1 h (4° C.). The supernatant was discarded and the fibril pellet was re-suspended in fresh PBS (typically ˜100 μL for unlabeled fibrils). For biotinylated fibrils, the pellet was resuspended in the same initial volume to achieve a nominal fibril concentration of 12.5 μM. Unlabeled fibrils were briefly sonicated for 30 seconds (three cycles of 10 seconds on and 30 seconds off) on ice and their concentration was determined by the BCA assay. Biotinylated fibrils were sonicated for 2 minutes (12 cycles of 10 secs on, 30 secs off) on ice before incubating with them with Streptavidin Dynabeads (Invitrogen, A11047). For fibril bead preparation for sorting, 10% biotinylated fibrils (6 μM) were mixed with 10⁷ beads in a final volume of 400 μL in PBSB (PBS with 1 mg/mL BSA). For fibril bead preparation for antibody analysis, 1% biotinylated fibrils (1 μM) were mixed with 10⁷ beads in a final volume of 400 μL in PBSB.

### Antibody Library Generation

Antibody library genes (theoretical diversity of 1.1×10⁸) were prepared by PCR. Three degenerate oligos were designed which included diversity in LCDR1, LCDR3 and HCDR2. Four individual PCRs were performed on the AF1 scFv gene using the yeast surface display plasmid (23) as a template. Overlap PCR was then performed to combine DNA fragments with terminal primers. The PCR product was purified via a 1% agarose gel followed by gel extraction (Qiagen, 28706). The wild-type AF1 scFv plasmid was double digested with NheI-HF (NEB, R3131L) and XhoI (NEB, R1046L), treated with alkaline phosphatase (NEB, M0525L), and purified via a 1% agarose gel. The digested backbone was cut and purified with gel extraction kit. The scFv gene and digested backbone were ligated by homologous recombination in the EBY100 yeast strain (Saccharomyces cerevisiae) by electroporation, as described previously (23,81). The total number of transformants obtained were ˜10⁹.

### Yeast Surface Display and Sorting

Five rounds of magnetic-activated cell sorting (MACS) were performed against Aβ fibrils (10% biotinylated fibrils) immobilized on streptavidin beads. For round 1, yeast cells (10⁹) expressing antibodies were sorted first using negative selections (three times) against biotinylated disaggregated Aβ immobilized on streptavidin beads (10⁷ beads per round) in PBSB, as described previously (23). Next, the remaining yeast cells after negative selections were sorted against 10⁷ beads coated with Aβ fibrils in 1% milk+PBSB for 3 hours (room temperature). The yeast cells bound to fibril-coated beads were collected by magnetic separation, washed, and grown in low pH SD-CAA media (20 g/L of dextrose, 6.7 g/L of yeast nitrogen base without amino acids, 5 g/L of casamino acids, 16.75 g/L of sodium citrate trihydrate, 4 g/L citric acid). Dilutions were plated to estimate the number of cells collected for the selections against Aβ fibrils. For round 2, the sorting was performed in a similar way except for using a reduced number of yeast cells (10⁷ cells).

For rounds 3, 4 and 5, the sorting was performed in similar way as round 2 except the negative selections were performed against IAPP fibrils (10% biotinylated IAPP fibrils immobilized at same concentration as 10% biotinylated Aβ fibrils). IAPP and biotinylated IAPP peptide were dissolved in HFIP at 1 mg/mL, aliquoted, and frozen at −80° C. For fibril assembly, peptides were thawed followed by snap freezing in liquid nitrogen and lyophilization. The lyophilized peptide was dissolved in 20 mM Tris (pH 7.4) (typically 150 μL.) and centrifuged at 21000× g for 10 minutes to remove any aggregates. The supernatant (typically 145 μL) was taken and moved to a new tube. The peptide concentration was determined by measuring the absorbance at 280 nm. Fibrils were assembled at 32 μM doped with 10% biotinylated peptide at 37° C., 300 RPM for 3-4 days. Post assembly, fibrils were purified by ultracentrifugation at 221000× g for 1 hour at 4° C. The fibril pellet was re-suspended back to the same final volume to achieve fibrils at 32 μM. For beads preparation, fibrils were sonicated for 2 minutes (10 seconds on, 30 seconds off) on ice followed by mixing with streptavidin beads (6 μM fibrils with 10⁷ beads in a final volume of 400 μL)

### Deep Sequencing and Data Analysis

Yeast plasmids containing the scFv gene were extracted from grow outs of the sorted antibody libraries from rounds 2 through 5 using a Zymoprep Yeast Plasmid Miniprep II Kit (D2004; Zymo Research). PCR was used to amplify a portion of the scFv containing LCDR1, LCDR3, and HCDR2 and to add Illumina adapter regions as well as DNA barcodes. This product was run on a 1% agarose gel and purified using a QIAquick Gel Extraction Kit (28704; Qiagen, Germantown, MD). A second PCR was performed with 2 μL of the purified product using primers that anneal to the Illumina adapter regions. This product was again purified via the gel extraction kit. The samples were sequenced using an Illumina MiSeq with a 300 bp paired-end sequencing reaction.

To analyze the paired-end output .fastq files, the two .fastq files corresponding to each sample were merged into one fastq file using BBMerge with the qtrim parameter set to 15 (82). The resulting file was converted to a fasta file and each line was analyzed. The lines containing a sequence were checked to ensure it was the correct length (540 bp) and that there were not bases called as ‘N.’ If so, it was translated using BioPython (83). If the resulting translation did not contain stop codons and started with the correct amino acid (T), it was further analyzed. Otherwise, the reverse complement of the sequence was translated and checked for starting amino acid and stop codons. Next, the eleven residues with potential mutations in the sequences were identified and added to a dictionary if they were previously unobserved or increased their count of observation. This process was repeated for every sample and the results were recorded in a .csv file. To select clones for experimental evaluation, ‘mutational analysis’ was conducted. For example, for a given set of potential mutations (e.g., DH61G, DL28N, NL30Y, and AL34T in LCDR1), all the observed clones were collected that contain those mutations (potentially among others) as well as all the clones with wild-type in those positions (irrespective of other mutations), and spearman correlation coefficient was calculated between the enrichment ratio of these clones and the frequency of mutation. The set of clones analyzed were all the clones that were common to five samples (Aβ fibrils, Aβ monomer, display antibody, polyspecificity reagent, and input) in both round 4 and 5. Mutational analysis was then conducted for one to nine mutations with the common set of clones. A requirement was that there were at least ten clones in the mutant and wild-type sets. Moreover, a p-value <0.05 for the Spearman correlation coefficient was required.

### Mammalian Plasmid Cloning, Expression, and Purification

Antibody sequences selected from deep sequencing analysis were ordered as separate VL and VH geneblocks. The geneblocks were combined by overlap PCR with primers containing NheI (forward primer) and HindIII (reverse primer) restriction sites. The PCR products were run on 1% agarose gels and purified via a Qiagen gel extraction kit. The purified DNA fragment was then double digested by Nhel-HF and HindIII-HF (NEB, R3104L) and further purified by a PCR clean-up. HEK 293-6E mammalian expression plasmid was double digested with NheI-HF and HindIII-HF followed by alkaline phosphatase treatment. The digested backbone was then gel purified by running it on a 1% agarose gel. Insert and backbone were ligated by T4 DNA ligase (NEB, M0202L), and the ligation mixture was transformed into competent DH5a cells and plated on LB agar plates supplemented with 100 μg/mL ampicillin. Single colonies were picked, grown in LB supplemented with ampicillin, mini-prepped (Qiagen, 27106), and sequences were confirmed.

For antibody expression, plasmids (15 μg) were mixed with PEI (45 μg) in F17 media (Invitrogen, A1383502) and incubated at room temperature for 10-20 minutes after vortexing briefly. The resulting mixture was then added to cells growing in F17 media supplemented with L-glutamine (Gibco, 25030081), Kolliphor (Fisher, NC0917244), and antibiotic G418 (Gibco, 10131035). Yeastolate feed (BD Sciences, 292804) was added at 20% w/v after 24-48 hours. The expression was continued for 4-5 days, and media was collected by centrifuging cells at 3500× g for 40 minutes. The media was transferred to a new tube and 1 mL of Protein A resin (Pierce, 20333) was added. Media and beads were rocked gently overnight at 4° C. Beads were collected by passing media through a filter column (Thermo Fisher Scientific, 89898) under vacuum. Beads were washed with 50-100 mL of PBS and protein was eluted from the beads in 0.1 M glycine (pH 3). Protein was then buffer exchanged into 20 mM acetate (pH 5) using Zeba desalting column (Thermo Fisher Scientific, 89894), passed through 0.2 μm filter (EMD Millipore, SLGV004SL), aliquoted, and stored at −80° C. Protein concentrations were determined by measuring the absorbance at 280 nm, and purity was evaluated by SDS-PAGE (Invitrogen, WG1203BOX).

### Analytical Size-Exclusion Chromatography

The purity of antibodies after the first purification step (Protein A) was also evaluated using size-exclusion chromatography (SEC). A Shimadzu Prominence HPLC System was used that was outfitted with a LC-20AT pump, SIL-20AC autosampler, and FRC-10A fraction collector. Antibodies in 20 mM acetate (pH 5) were buffer exchanged into PBS (pH 7.4). For analytical SEC, 100 μL of antibodies (diluted to 0.1 mg/mL) were loaded onto SEC column (GE, 28990944; Superdex 200 Increase 10/300 GL column) and analyzed at 0.75 mL/min with using a PBS running buffer supplemented with 200 mM arginine (pH 7.4). Absorbance was monitored at 220 and 280 nm, and the 280 nm were primarily used for analysis. The % antibody monomer was evaluated by analyzing the area under the monomeric peak (excluding times before seven minutes and after 22 minutes). In some cases, the antibodies were purified using SEC after Protein A purification. In these cases, the peak times of fraction collection were chosen based on analytical runs. Antibody fractions were collected, buffer exchanged into PBS (pH 7.4), filtered, aliquoted, and stored at −80° C.

### Antibody Binding Analysis

For affinity analysis, the binding of antibodies to Aβ fibrils was evaluated using streptavidin dynabeads and flow cytometry. Beads were immobilized with 1% biotinylated fibrils as described above. The fibril-coated beads were washed twice with PBSB and then blocked with 10% milk in PBS at room temperature for 1 hour with end-over-end mixing. Afterward, the beads were washed 2× with PBSB.

Antibodies were thawed and centrifuged at 21000× g for 5 minutes to remove aggregates. The supernatant was transferred to a new tube and the antibody concentration was determined by measuring absorbance at 280 nm. Antibody dilutions were made in PBSB. Fibril-coated beads (1.25×10⁵ beads per evaluated antibody concentration) were incubated with antibodies in 96 well plates (Greiner, 650261) in 1% milk for 3 hours at 25° C.(300 RPM). Next, the plates were centrifuged at 3500 RPM for 5 minutes, the supernatants were discarded, and the beads were washed once with ice-cold PBSB. After washing, the plates were spun down again and the beads with resuspended with 300× diluted goat anti-human Fc AF647 (Jackson Immunoresearch, 109-605-098) on ice for 4-5 minutes. Beads were then washed once more with ice-cold PBSB and analyzed via flow cytometry using a BioRad ZE5 Analyzer Blank streptavidin beads were also blocked with 10% milk in PBS and treated in the same way as fibril beads as controls. Two independent repeats were performed with different batches of beads coated with Aβ fibrils.

For antibody conformational specificity analysis, the experiments were performed in the same way as described above, except that the antibodies were pre-incubated with disaggregated (non-biotinylated) Aβ. Antibody binding analysis was performed in 1% milk at a fixed antibody concentration (30 nM) and a range of disaggregated Aβ concentrations. The antibody binding results were normalized to the average value obtained without disaggregated Aβ. Two independent repeats were performed with different batches of beads coated with Aβ fibrils.

### Antibody Epitope Analysis

Fibrils were also assembled using Aβ peptides with N-terminal deletions including Ab2-42 (Bachem, 40306028.0500), Ab3-42 (Bachem, 4090137.0500), Ab4-42 (Bachem, 4090138.0500), Ab5-42 (Bachem, 4041241.0500), and Ab11-42 (Anaspec, 63317) in addition to Aβ1-42, and purified using ultracentrifugation. Fibrils were then spotted on nitrocellulose membranes at equal Thioflavin T florescence. Membranes were blocked with 5% milk in PBS at room temperature for 1 hour followed by 3× washing with PBST (PBS with 0.1% Tween 20). Membranes were then incubated with Aβ antibodies at 10 nM (1% milk) in PBST at room temperature for 2-3 hours. Following primary incubation, membranes were washed 3× with PBST followed by incubation with goat anti-human Fc IgG HRP ( 1/5000× dilution, Invitrogen, A18817) in PBST at room temperature (1 hour). Following secondary incubation, the blots were washed 3× with PBST, developed with ECL (Pierce, 32109) and imaged with a Biorad imager.

### Polyspecificity Analysis

The polyspecificity reagent (PSR) was prepared as previously described (21). CHO cells (10⁹, Gibco, A29133) were pelleted, the cell pellets were washed separately with PBSB and Buffer B (50 mM HEPES, 0.15 M NaCl, 2 mM CaCl₂, 5 mM KCl, 5 mM MgCl₂, 10% Glycerol, pH 7.2), and then pelleted again. The pellets were resuspended in 5 mL of Buffer B supplemented with a protease inhibitor (Sigma Aldrich, 4693159001). Next, the resuspended cells were homogenized for 90 seconds (three cycles of 30 seconds) followed by sonication for 90 seconds (three cycles of 30 seconds). The cell suspension was then spun down at 40000× g for 1 hour and the supernatant was discarded.

The pellet, comprising the enriched membrane fraction (EMF), was resuspended in Buffer B with a Dounce homogenizer for 30 strokes. The protein concentration was determined using a detergent compatible protein assay kit (BioRad, 5000116). The EMF was diluted to a theoretical concentration of 1 mg/mL in solubilization buffer (50 mM HEPES, 0.15 M NaCl, 2 mM CaCl₂, 5 mM KCl, 5 mM MgCl₂, 1% n-dodecyl-β-D-maltopyranoside (Sigma Aldrich, D4641), protease inhibitor and pH 7.2)) and mixed overnight (end-over-end) at 4° C. The soluble membrane protein fraction was centrifuged at 40000× g for 1 hour and the supernatant was collected. The final concentration of supernatant was ˜0.8-0.9 mg/mL.

Sulfo-NHS-LC-biotin (Thermo Fisher, PI21335) was dissolved in distilled water at ˜11.5 mg/mL. The stock solution of Sulfo-NHS-LC-biotin (150 μL) and the PSR reagent (4.5 mL at 0.8-0.9 mg/mL) were mixed via end-over-end mixing at room temperature (45 minutes). The reaction was quenched (10 μL of 1.5 M hydroxylamine at pH 7.2), and biotinylated PSR (b-PSR) was aliquoted and stored at −80° C.

Protein A magnetic beads (Invitrogen, 88846) were washed twice and incubated with antibodies in 96 well plates (VWR, 650261) overnight at 4° C. The antibodies were purified either via one-step (Protein A) or two-step (Protein A followed by SEC) purification methods. Next, the antibody coated beads were washed by centrifuging the 96 well plates at 3500× g for 4 minutes and washed twice with PBSB. Afterward, the beads were resuspended with a 10× diluted solution of biotinylated PSR and incubated on ice for 20 minutes. Beads were washed once with PBSB and incubated with 1000× diluted solution of streptavidin AF-647 (Invitrogen, S32357) and 1000× diluted solution of goat anti-human Fc F(ab′)2 AF-488 (Invitrogen, H10120) on ice (4 minutes). Beads were washed once, resuspended in PBSB, and analyzed via flow cytometry. The antibody binding steps were performed in PBSB, and three independent repeats were performed. The control antibodies used were the variable regions of crenezumab, elotozumab, duligotuzumab and emibetuzumab grafted onto a common IgG1 framework. The control antibodies were two-step purified (Protein A and SEC).

### Immunoblotting and Western Blotting

For immunoblots using synthetic Aβ peptides, disaggregated Aβ and unlabeled Aβfibrils were prepared as discussed above. Disaggregated Aβ, and fibrils of Aβ, IAPP and α-synuclein were spotted on nitrocellulose membranes. Membranes were allowed to dry for at least 1 hour at room temperature before use. Membranes were blocked with 5% milk in PBS for 1 hour at room temperature. Afterward, the membranes were washed 3× using PBST (PBS with 0.1% v/v Tween 20) with rocking (5 minutes). Antibodies were thawed, centrifuged, and their concentrations were determined via absorbance measurements at 280 nm. Antibody binding was performed at 10 nM in PBS with 1% at room temperature (3 hours). Next, the membranes were washed 3× with PBST and incubated with a 7500× diluted solution of goat anti-human Fc HRP (Invitrogen, A18817) at room temperature for 1 hour. Following secondary incubation, the blots were washed 3× with PBST and developed with ECL (Pierce, 32109). The signals were evaluated using X-Ray film (Thermo Scientific, 34090) and the films were developed Three independent repeats were performed for all experiments.

### Mouse Models

This study was conducted in a facility approved by the American Association for the Accreditation of Laboratory Animal Care, and all experiments were performed in accordance with the National Institutes of Health Guide for the Care and Use of Laboratory Animals and approved by the Institutional Animal Care and Use Committee of the University of Michigan. Mice were housed at the University of Michigan animal care facility and maintained according to U.S. Department of Agriculture standards (12 hours light/dark cycle with food and water available ad libitum). 5×FAD mice (B6.Cg_Tg(APPSwFILon,PSEN1*M146L*L286V) 6799Vas/Mmjax; The Jackson Laboratory MMRRC stock #034848) expressing human amyloid precursor protein (APP) and presenilin-1 (PSEN1) with five AD mutations: the Swedish (K670N/M671L), Florida (1716V), and London (V7171) APP mutations and the M146L and L286V PSEN1 mutations and non-transgenic littermates (courtesy of Geoffrey Murphy, University of Michigan) were euthanized at 8 months (for immunofluorescence analysis) and 22-24 months (for immunoblots and western blots) for brain collection.

### Tissue Harvesting

Animals were deeply anesthetized with isofluorane and perfused transcardially with 1× PBS. Brains were divided sagittally. One half was immediately placed on dry ice and stored at −80° C. for biochemical studies while the other half was fixed in 4% paraformaldehyde at 4° C. for 24 hours, and cryoprotected in 10% and 30% sucrose solutions in 1×PBS at 4° C. until saturated. Fixed hemispheres were snap frozen in OCT medium and sectioned at 12 μm sagittally using a cryostat and sections were stored at −20° C. for immunofluorescence.

### Mouse Brain Immunoblots and Western Blots

The 5×FAD and non-transgenic littermate forebrain samples were homogenized in PBS with a protease inhibitor cocktail (Sigma Aldrich, 11873580001) using a 1:3 dilution of tissue: PBS (w/v). Samples were centrifuged at 9300× g for 10 minutes at 4° C. Pellets were resuspended in PBS with protease inhibitor cocktail (Roche), centrifuged at 9300× g for 10 minutes (4° C.), and supernatants were discarded. The pellet was resuspended in RIPA buffer with protease inhibitor, vortexed (1 minute), and incubated at room temperature (1 hour) Samples were sonicated (water bath sonicator) for 5 minutes and centrifuged for 30 minutes (16000× g at 4° C.). RIPA fractions of brain extracts (7 μg of total protein) were spotted directly onto nitrocellulose membranes and allowed to dry (1 hour). Control dot blots (loading controls) were stained with Ponceau S (5 minutes) and washed 3× with distilled water. The other dot blots were blocked with 10% nonfat dry milk in TBS-T buffer at room temperature (1 hour). Each dot blot was then incubated with antibodies at 50 nM (1% nonfat dry milk in TBST) overnight at 4° C. Next, the blots were washed with TBST and incubated with a 5000× diluted solution of HRP- conjugated goat anti-human IgG at room temperature for 1 hour. Afterward, the blots were washed with TBST and developed using Ecobright Nano HRP Substrate (Innovative Solutions) and visualized with the Genesys G:Box imaging system (Syngene). Three independent repeats were performed.

For western blotting, 50 μg of total protein was loaded on precast NuPAGE 4-12% Bis-Tris gels (Invitrogen, WG1402A). Gels were subsequently transferred onto nitrocellulose membranes and first stained with Ponceau S and washed 3× with distilled water. After imaging, membranes were destained for 1 minute with 0.1 M NaOH and washed 3× with distilled water. Next, membranes were blocked for 1 hour at room temperature with 10% nonfat dry milk in TBST buffer. Membranes were probed overnight at 4° C. with aducanumab (Adu) and NAB (Sigma-Aldrich, A3854) at 100 nM in TBST with 1% milk or 100 nM antibody (clone 93 or 97) in 1% nonfat dry milk in TBST. HRP-conjugated goat anti-human/mouse IgG (5000× dilution) HRP was used for detection. Ecobright Nano HRP Substrate (Innovative Solutions) was used to visualize bands with the Genesys G:Box imaging system (Syngene). Three independent repeats were performed.

### Immunofluorescence Analysis of Mouse Brain Samples

Fixed brain sections were post-fixed for 10 minutes in methanol at 4° C. Sections were washed in 1× PBS three times for 10 min and subjected to heat-induced antigen retrieval in 10 mM citrate buffer (pH 6) Sections were washed in 1× PBS two times for 5 minutes and permeabilized with 0.5% Triton-X 100, washed for 10 minutes in 1× PBS, and blocked using the Mouse on Mouse (M.O.M.) Mouse IgG Blocking Reagent (M.O.M. Immunodetection Kit, Vector, BMK-2202) for 1 hour. Sections were washed 2× for 2 minutes in 1× PBS and incubated for 5 minutes in M.O.M. diluent Sections were then incubated with Aβ antibodies aducanumab or 97 (200 nM) and NAB (200× dilution) in M.O.M. diluent overnight at 4° C. The following day, sections were washed in 1× PBS three times for 10 minutes each and incubated with goat anti-mouse IgG Alexa-488 (Invitrogen; 1:500) and goat anti-human IgG Alexa-647 (1:500) for 1 hour. Sections were then washed in PBS 3× for 10 minutes each and incubated with DAPI (Sigma) to label nuclei for 5 minutes at room temperature, washed 3× for 5 minutes each, and were mounted with Prolong Gold Antifade Reagent (Invitrogen). Slides were imaged using a Leica SP5 Confocal microscope.

### Example 1

This example describes antibody library design and identification of affinity-matured antibody candidates.

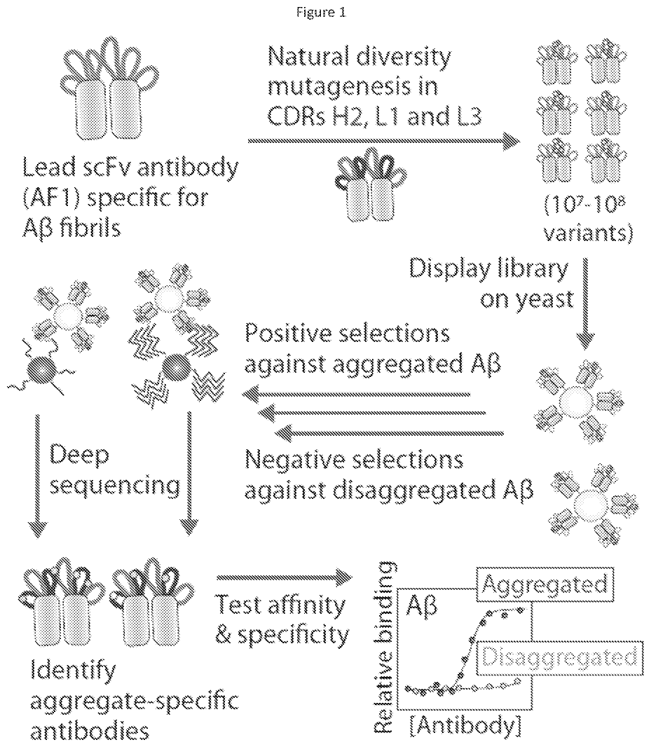

A strategy for systematic affinity maturation of a lead anti-Aβ amyloid antibody is summarized in FIG. 1. The first step in this process is to design an antibody library that preserves that antigen recognition activity of the lead antibody (AF1) while identifying sites in the CDRs for affinity maturation. Given that AF1 was generated by directed mutagenesis in heavy chain CDR3, sites were identified in the other five CDRs for further mutagenesis. However, there are a large number of potential CDR sites to mutate (54 positions in the five CDRs) and a very large number of possible antibody variants (>1070 variants).

To limit the library design to a size that can be evaluated using standard display methods such as yeast surface display (˜10⁷-10⁹ variants), the design involved identifying the most attractive subset of CDR sites and subset of residues per site that met a number of design criteria. First, it was reasoned that the most naturally diverse sites in human antibodies are the most attractive for mutagenesis because they are most likely to be solvent exposed and positioned for productive engagement of the antigen while being least likely to adversely impact stability. Only CDR sites were considered in which the most common residue on average in human antibodies, as judged by the AbYsis database of tens of thousands of human antibodies (37), was present at a frequency of <50%.

Next, the remaining CDR sites were prioritized for mutagenesis with the goal of sampling combinations of four to six residues per site that included the wild-type residue and combinations of residues expected to lead to high antibody specificity in addition to high affinity. The lead AF1 clone possessed five Asp and five Tyr residues in the heavy chain CDR3 (HCDR.3), and it was previously found that removal of either type of residue from this CDR reduced specificity (38). Therefore, sites in the other CDRs were identified that were compatible with encoding the wild-type residue and at least one of these residues (Asp or Tyr), as well as other residues that are most common in human antibodies using degenerate codons. Third, any degenerate codons that included positively charged residues (Arg, Lys and His) were eliminated because it has been shown that excessive positive charge in the antigen-binding site is linked to increased risk for non-specific interactions (40-46). Any degenerate codons that encoded stop codons also were eliminated and the number of Cys-encoding codons were minimized while not completely eliminating them. The reason for not completely excluding Cys is because it is encoded by degenerate codons that include combinations of common CDR residues in human antibodies such as Gly, Tyr and Asp. Fourth, degenerate codons were selected that maximized the sum of the average frequencies of each residue in human antibodies to maximize coverage of the natural amino acid diversity of human antibodies.

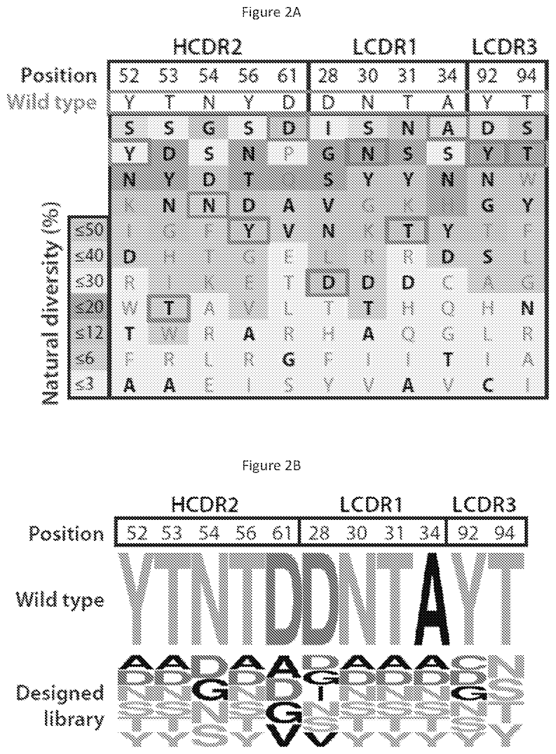

The library design is shown schematically in FIGS. 2A and 2B. Eleven sites for mutagenesis in three CDRs were identified: four sites in light chain CDR 1, five sites in heavy chain CDR2 and two sites in light chain CDR3. At each site, the wild-type residue is boxed in red and the three to five mutations are highlighted as bolded black font in FIG. 2A. At each site, the residues are listed in order of most common on average in human antibodies (top) to least common (bottom). For example, at position 52 in heavy chain CDR2, the wild-type residue (Tyr) was sampled along with five other residues that included Asp, two residues common in human antibodies at this position (Ser and Asn), and two residues that are less common but required because of the constraints of degenerate codons. Using a similar strategy at the other ten CDR sites (FIG. 2B), the resulting designed library contained 1.1×10⁸ theoretical variants.

The antibody library was then generated, displayed on the surface of yeast as Aga2 fusion proteins, and sorted against Aβ42 fibrils immobilized on magnetic beads (FIG. 3). To maximize antibody specificity, three negative selections were performed per round of selection to remove non-specific antibodies before performing positive selections against Aβ fibrils. In rounds 1 and 2, negative selections were performed against disaggregated (immobilized) Aβ to maximize conformational specificity. In rounds 3-5, negative selections were performed against islet amyloid polypeptide (IAPP) fibrils to maximize sequence specificity. After five rounds of sorting, strong enrichment was observed in terms of the percentage of yeast cells that bound to fibrillar Aβ relative to control selections performed against disaggregated Aβ (FIG. 3A). The ratio of the number of yeast cells retained against fibrillar Aβ relative to that for disaggregated Aβ was >100 after five rounds of selection (FIG. 3B).

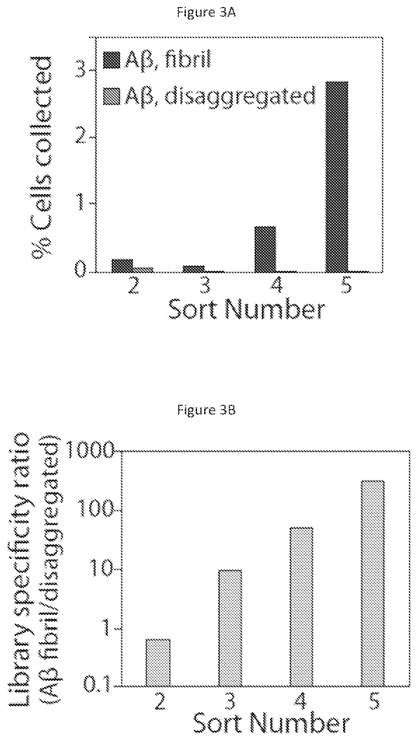

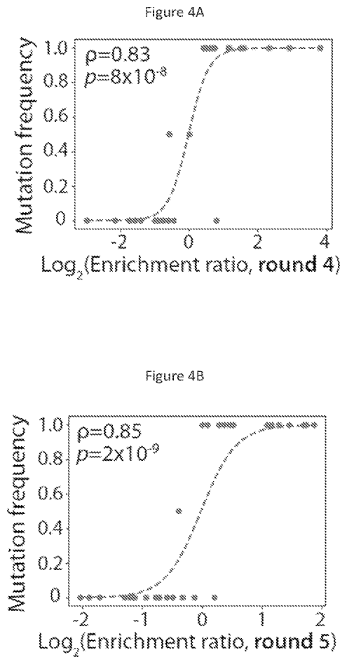

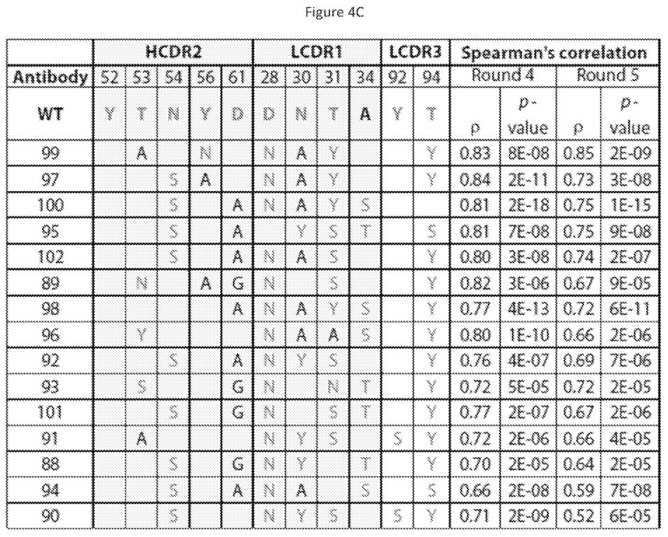

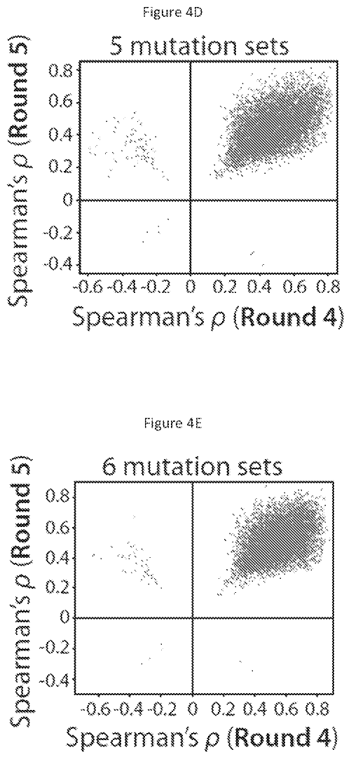

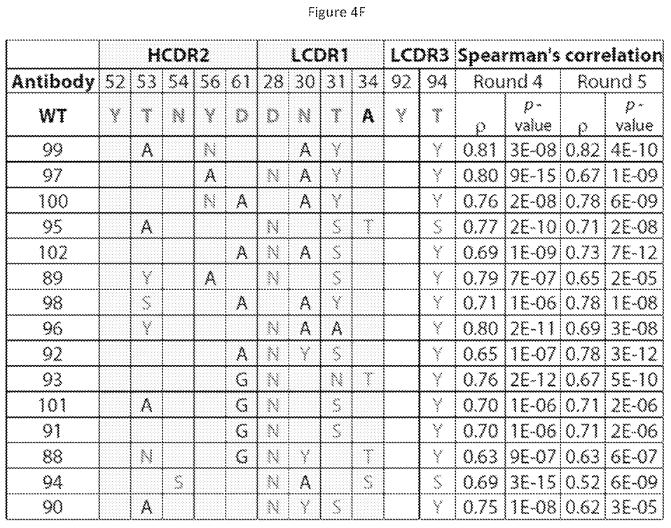

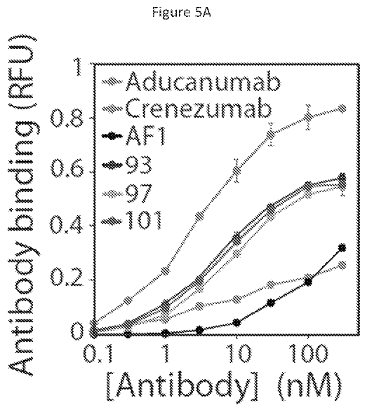

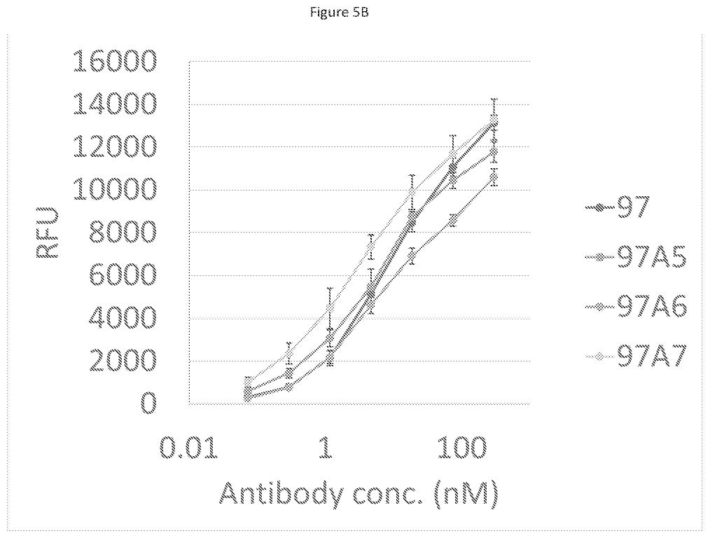

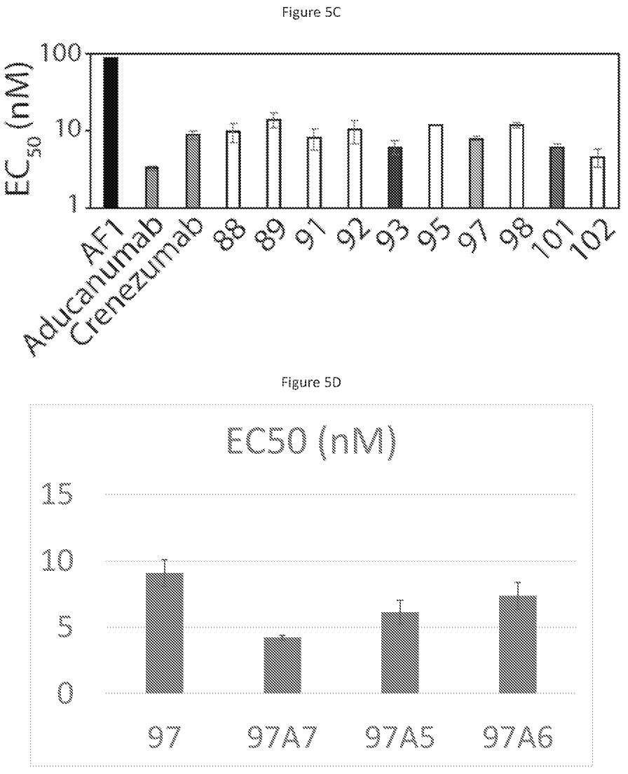

These promising sorting results led to sequencing of the sorted antibody libraries before and after rounds 4 and 5 to better understand mutations most strongly correlated with improved antibody binding (FIGS. 4A-4C). 7464 unique antibodies were identified using deep sequencing and correlations were evaluated between individual mutations or sets of mutations and enrichment ratios for antibody variants with such mutations. Therefore, the Spearman correlation coefficients were evaluated for all possible single and multiple sets of mutations by comparing the enrichment ratios for all antibody variants with either wild-type or mutant residues at these sites regardless of the residues at the other sites. While significant sets of mutations were identified when considering as few as one mutation and as many as nine mutations (the maximum evaluated), it was found that sets of five and six mutations led to the best combination of a relatively large number of mutant (>10) and wild-type (>10) antibodies per set of mutations, high Spearman correlation values (p>0.5) and high statistical significance (p-value<0.001). Moreover, Spearman correlation coefficients were well correlated between rounds 4 and 5, which demonstrates that the deep sequencing results were consistent between multiple rounds of sorting (FIGS. 4D and 4E).

For example, a set of six mutations (T53A and Y56N in HCDR1, D28N, N30A and T31Y in LCDR1, and T94Y in LCDR3) were evaluated by identifying all antibody variants that had these mutations (16 variants) or wild-type residues (16 variants) at these positions regardless of their residues at the other five mutated sites (FIG. 4A). This set of mutations resulted in large, positive, and highly significant Spearman correlation coefficients in both rounds 4 (ρ=0.83 and p-value of 8×10⁻⁸) and 5 (p=0.85 and p-value of 2×10⁻⁹). Antibody variants with these mutations were expected to display improved antibody affinity.

Several other sets of six mutations were observed that also displayed favorable Spearman correlations, and antibody variants with these mutations were selected for further analysis (FIG. 4B). Sets of five mutations also were identified with favorable Spearman correlation coefficients that corresponded to these same antibody variants (FIG. 4F).

### Example 2

This example demonstrates that selected antibody variants display increased affinity and high conformational specificity for Aβ fibrils.

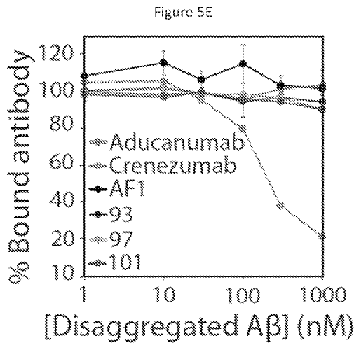

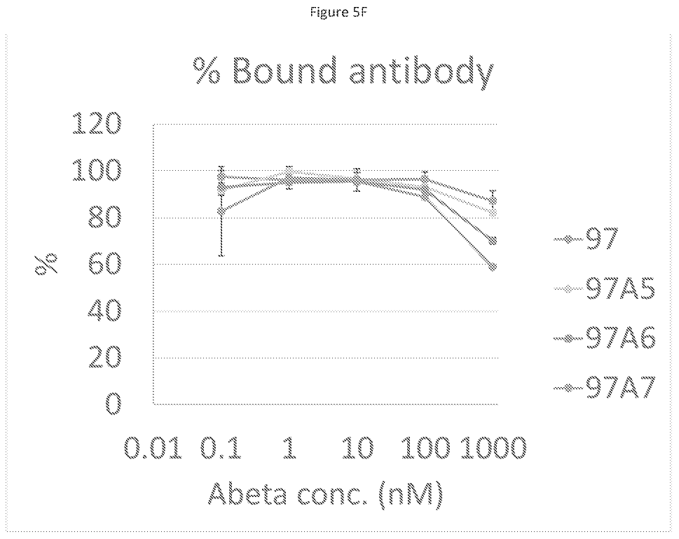

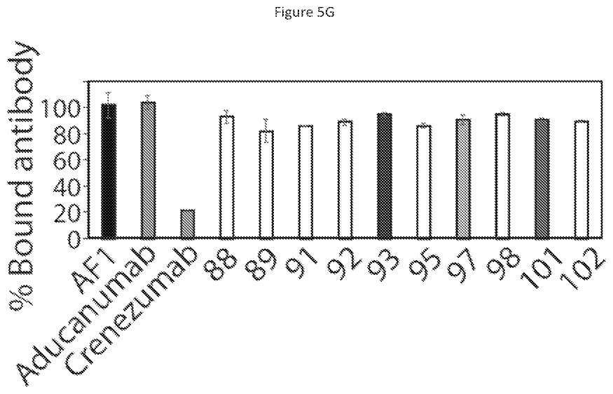

The antibodies selected in Example I were engineered as Fc-fusion proteins and their affinities and conformational specificities were evaluated. To this end, the selected antibodies were directly compared to two clinical-stage antibodies specific for Aβ, namely aducanumab and crenezumab. Aducanumab recognizes an N-terminal Aβ epitope (residues 3-7), and selectively recognizes Aβ fibrils and oligomers relative to disaggregated Aβ (15, 47). In contrast, crenezumab recognizes a central Aβ epitope (residues 13-24) and binds to both aggregated and disaggregated Aβ (15, 47). The variable domains of each clinical-stage antibody were grafted onto an IgG1 scaffold with a human Fc, which resulted in differences in the antibody sequence outside the variable regions between the antibodies tested in this study and the actual clinical-stage antibodies (e.g., crenezumab is an IgG4 antibody). These antibodies are referred to herein by their common names despite these differences. The selected antibody clones and clinical-stage antibodies both expressed well (purification yield of >30 mg/L) and were isolated with high purity (FIGS. 5H and 5I).

The apparent affinity of the selected antibody variants relative to AF1 and the clinical-stage antibody controls (FIGS. 5A-5D) was evaluated. Modest affinity of AF1 for Aβ fibrils was observed (EC₅₀ of 99+2 nM). Notably, significant (i.e., order of magnitude) increases in affinity were observed for all of the selected antibody variants, and the EC₅₀ values (4-13 nM) were similar to crenezumab (9±1 nM) and modestly higher than aducanumab (3±0.2 nM).

It is relatively common to lose antibody conformational specificity during in vitro affinity maturation because of the acquisition of mutations that increase affinity via non-conformation specific binding mechanisms. Therefore, the affinity-matured antibodies were evaluated to assess if they retained conformational specificity (FIGS. 5E-5G). Specifically, the antibodies (30 nM) were pre-incubated with various concentrations of disaggregated Aβ and fibril-binding activity was evaluated. As expected, crenezumab displayed low conformational specificity and its binding to Aβ fibrils was inhibited by disaggregated Aβ. Conversely, aducanumab binding to fibrils was weakly inhibited by disaggregated Aβ, which is consistent with its high conformational specificity (14). Notably, the binding of the affinity-matured clones to Aβ fibrils was also weakly inhibited by disaggregated Aβ (82-99% bound antibody at 1000 nM disaggregated Aβ) and behaved similar to the parental antibody (AF1).

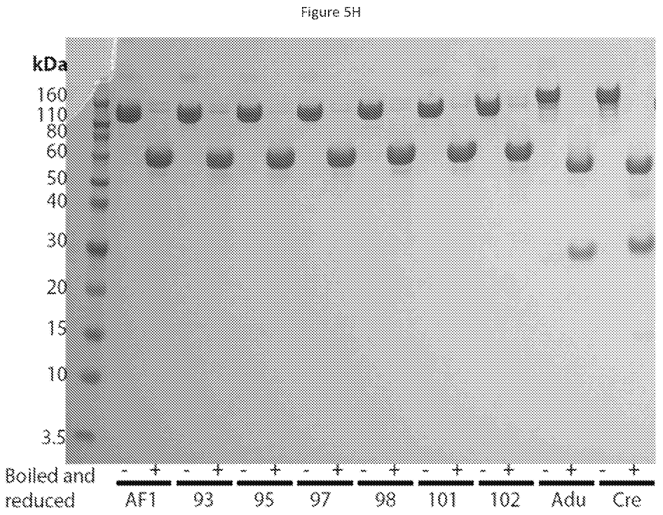

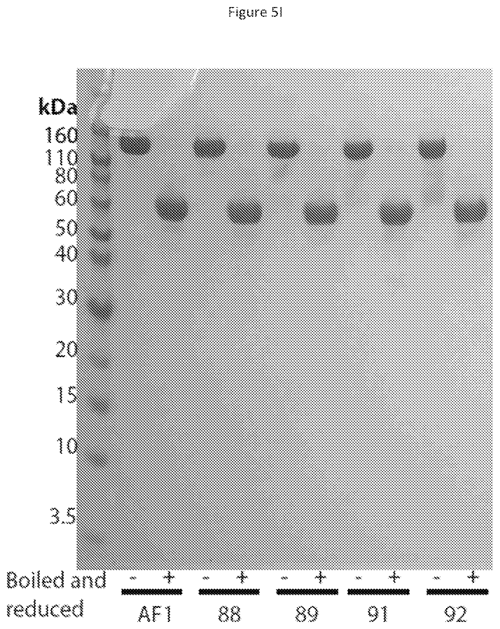

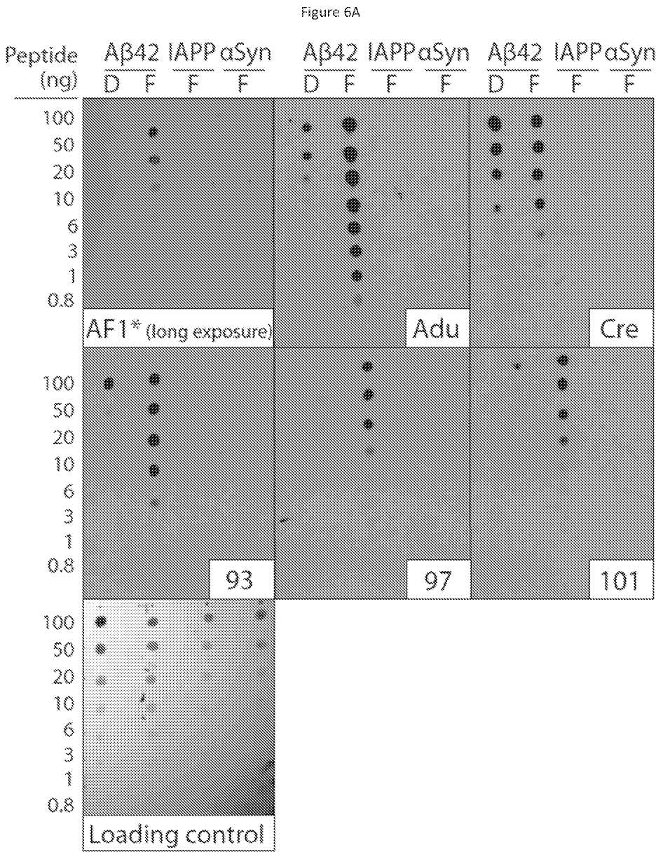

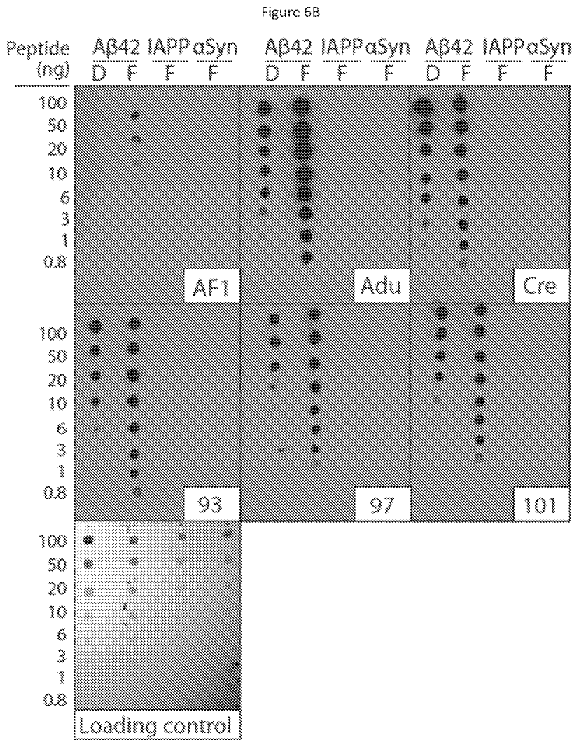

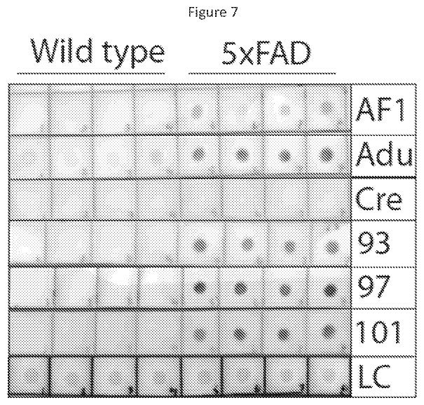

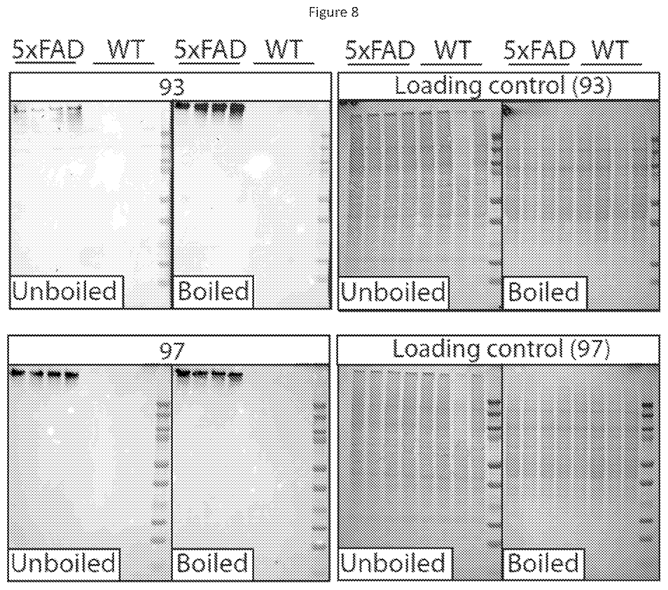

The conformational specificity of the selected antibodies was then assessed using immunodot blots (FIG. 6A). The parental antibody (AF1) displayed weak reactivity at 10 nM and required long exposure times (45 minutes) to detect signals for Aβ fibrils. Conversely, the clinical-stage antibody controls and the affinity-matured variants developed signals rapidly, with images reported are after short exposures (30 seconds) except for AF1. Aducanumab and the selected affinity-matured antibody variants (clones 93, 97 and 101) displayed relatively high conformational specificity. Moreover, crenezumab displayed little conformational specificity, as expected based on the results shown in FIG. 5. Longer exposures (45 minutes) for the clinical-stage and affinity-matured variants revealed additional binding to both fibrillar and disaggregated Aβ (FIG. 6B). To assess whether the affinity-matured antibodies recognize aggregated Aβ formed in vivo (FIG. 7), the antibodies were evaluated using immunodot blots of brain homogenates obtained from transgenic (5×FAD) and control mice. The parental antibody (AF1) displayed weak immunoreactivity with the 5×FAD samples, while the selected clones (93 and 97 and 101) displayed stronger and specific detection of 5×FAD samples from four mouse brains relative to those from four control mouse brains. Interestingly, aducanumab detected the 5×FAD samples and also weakly reacted with the wild-type samples, while crenezumab failed to detect either type of sample. These findings were confirmed for two selected antibodies (clones 93 and 97) using Western blotting, and strong and specific signals were detected for the 5×FAD samples (FIG. 8).

Overall, these results demonstrate that the affinity-matured antibodies displayed strong and specific recognition of Aβ aggregates both in vitro and in vivo, and compared favorably to clinical-stage Aβ antibodies.

### Example 3

This example demonstrates that the affinity-maturated antibodies displayed favorable specificity and biophysical properties.

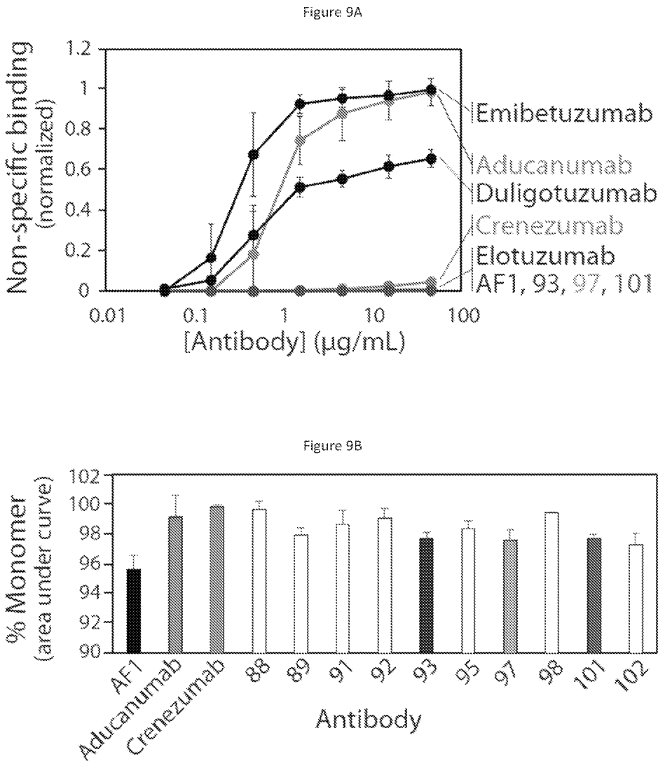

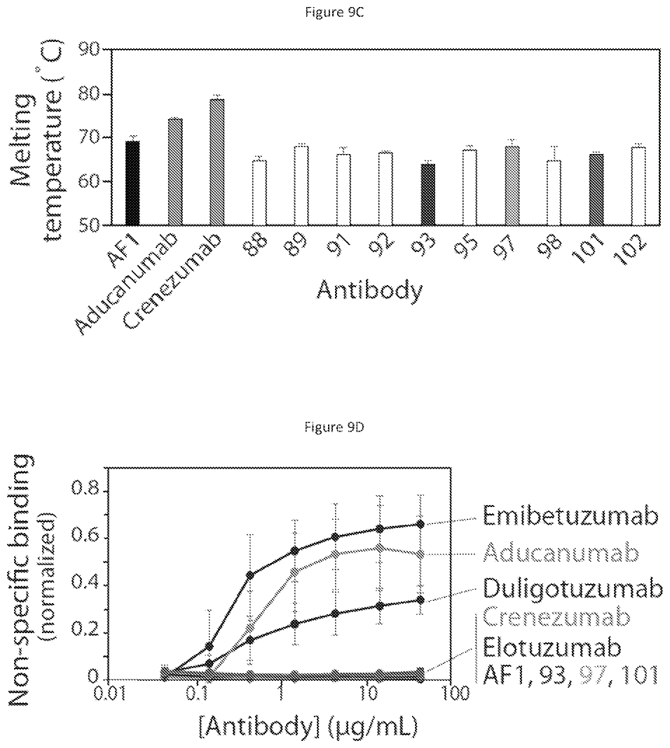

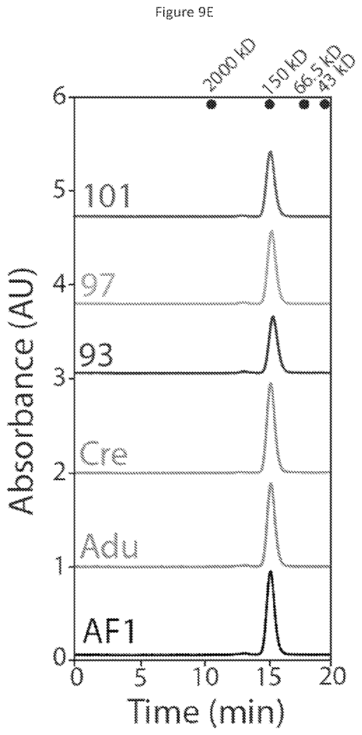

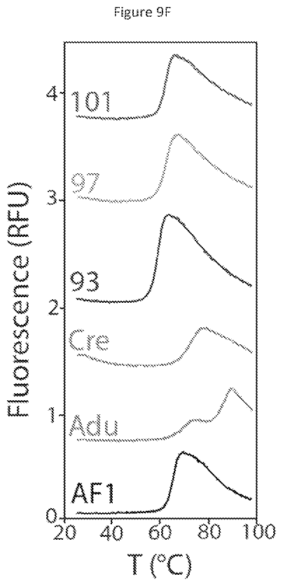

One of the most common limitations of using in vitro antibody discovery and engineering methods is the generation of antibodies with suboptimal biophysical properties, such as low stabilities, solubilities, and specificities, relative to antibodies generated by the immune system (36, 49-51). Therefore, the biophysical properties of the affinity-matured antibodies described above were analyzed to determine if they maintained favorable specificities and stabilities (FIG. 9A). Non-specific binding for the antibodies was assessed using a previously reported polyspecificity reagent (PSR) that is composed of soluble membrane proteins isolated from CHO cells (36,52). Antibody binding to this reagent is a strong indicator of the level of antibody specificity and the likelihood of abnormal pharmacokinetics (53). Encouragingly, the affinity-matured antibodies, including clones 93, 97 and 11, displayed extremely low levels of non-specific interactions that were similar to their parental antibody (AF1) and a control clinical-stage antibody with high specificity (elotuzumab) (36). Moreover, the affinity-matured antibodies were even more specific than crenezumab, which also displayed relatively low levels of non-specific binding. Interestingly, aducanumab displayed much higher levels of non-specific binding that were similar to the control clinical-stage antibodies with high levels of non-specific binding (emibetuzumab and duligotuzumab) (36). Although these results were performed using the affinity-matured antibodies after only one-step purification (Protein A) and the control clinical- stage antibodies after two-step purification (Protein A and size-exclusion chromatography), similar results were obtained for the affinity-matured antibodies after two-step purification (FIG. 9D).

The physical stabilities of the affinity-matured antibodies were also assessed (FIG. 9B and 9C). Antibodies with poor stability often display aggregation at low pH during elution from Protein A columns. Therefore, the percentage of monomeric antibody after Protein A purification was determined for the affinity-matured antibodies relative to the control clinical-stage antibodies (FIGS. 9B and 9E). Encouragingly, the affinity-matured antibodies displayed high levels of monomeric protein (95%) that were similar to the clinical-stage antibodies. The melting temperatures of the single-chain (scFv) antibodies relative to the clinical-stage IgGs (FIGS. 9C and 9F) were evaluated. Due to the lack of constant (CH1 and CL) domains, the single-chain antibodies were expected to have lower stabilities than the clinical-stage IgGs. The affinity-matured antibodies displayed high stabilities (Tₘ values of 64-69° C.) that were comparable to the parental antibody (AF1, Tₘ of 69° C.) and modestly lower than the clinical-stage IgGs (74-79° C.).

The results of this example demonstrate that the affinity-matured antibodies display a combination of biophysical properties that are favorable in comparison to clinical-stage Aβ antibodies.

### Example 4

This example demonstrates that additional affinity maturation of the anti-Aβ antibodies does not compromise conformational and sequence specificity.

It was evaluated whether the above-described methods could be used to further affinity mature one of the best antibody variants (clone 97) while maintaining high conformational specificity and low non-specific binding. A sub-library for clone 97 with mutations in heavy chain CDR1 and light chain CDR2 was designed and screened, as these two CDRs were the only ones not mutated during the initial round of discovery (heavy chain CDR3) and the first round of affinity maturation (heavy chain CDR2 and light chains CDRs 1 and 3).

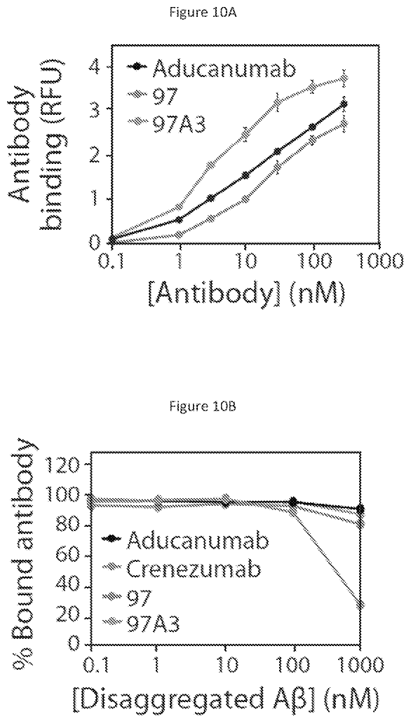

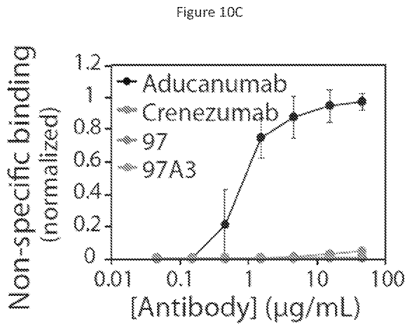

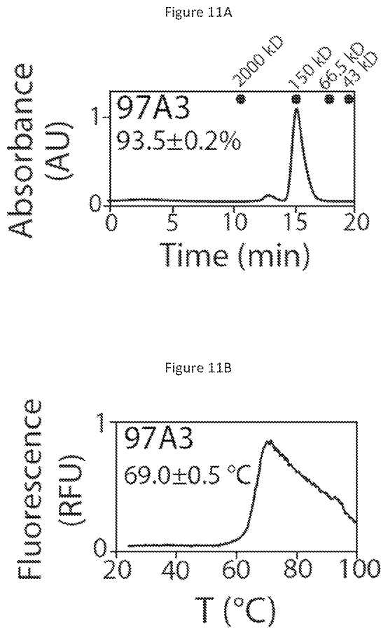

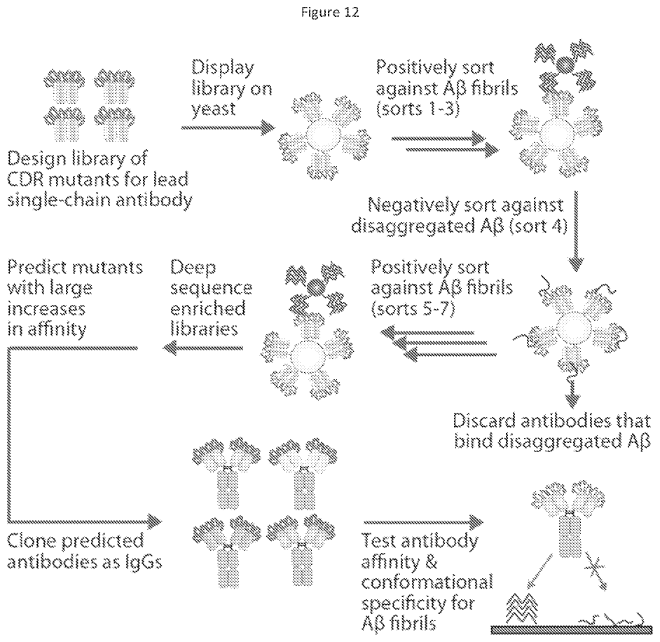

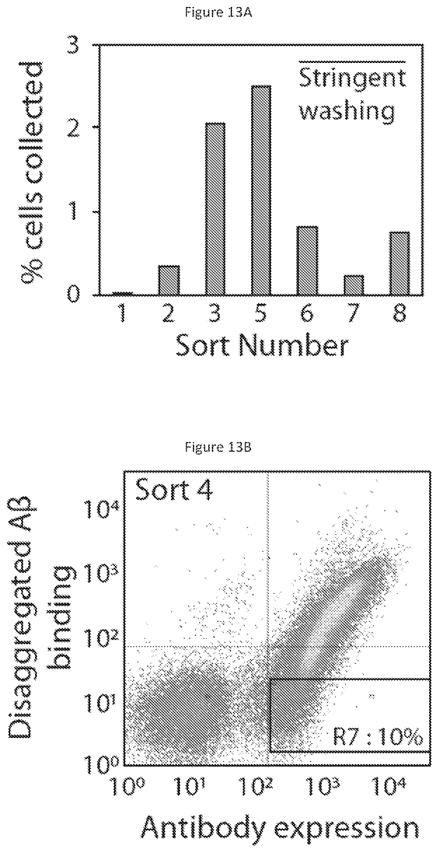

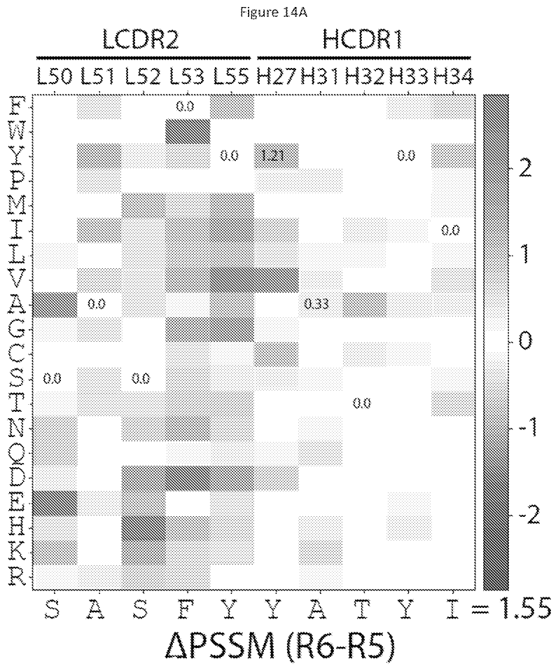

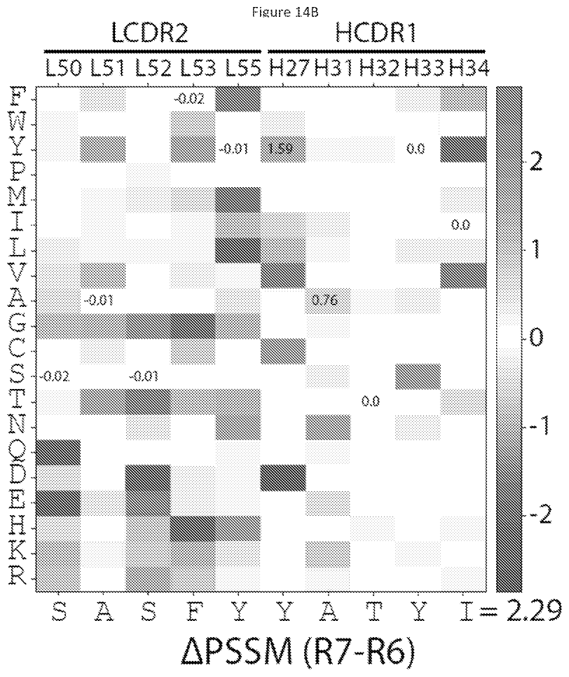

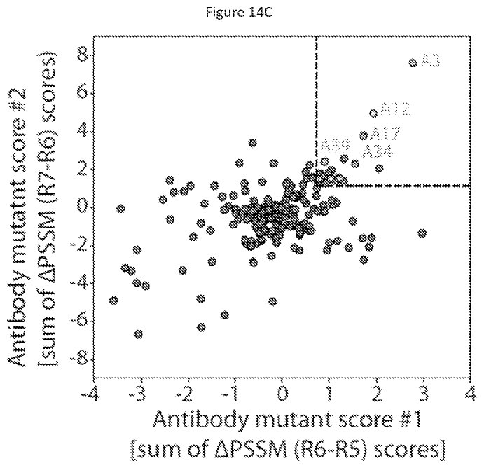

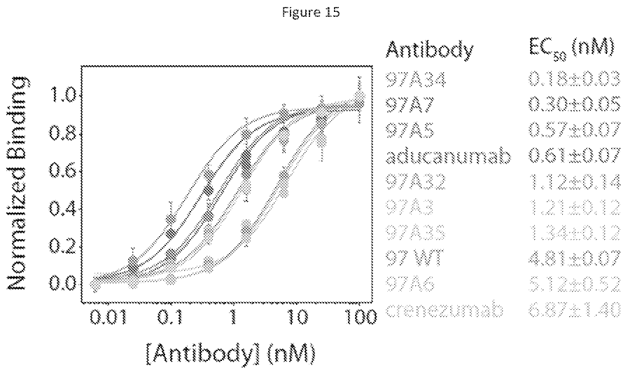

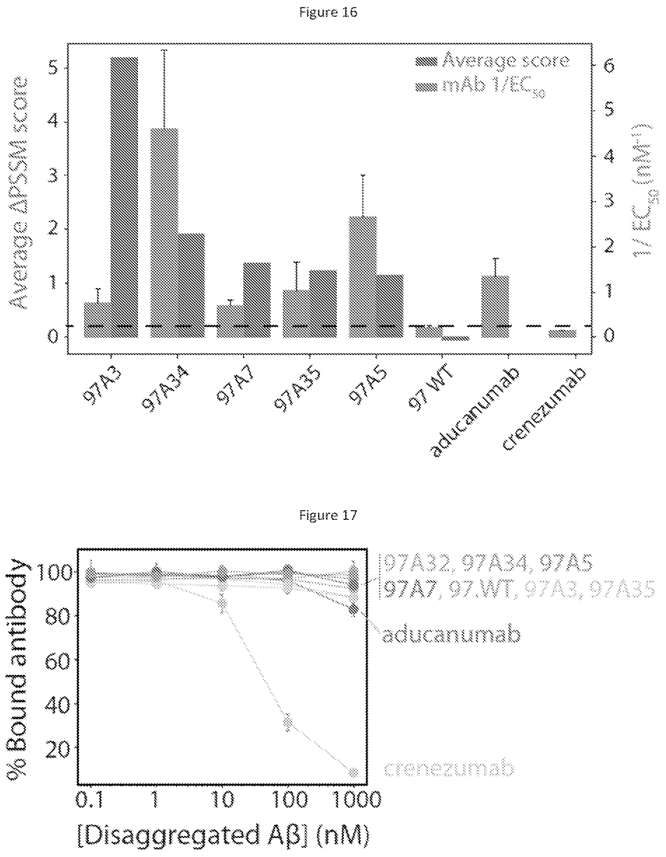

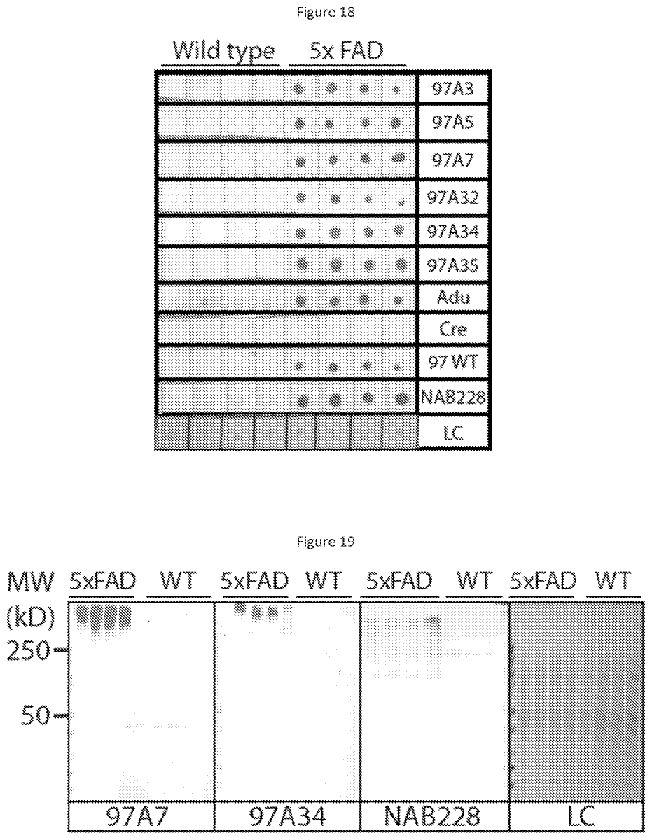

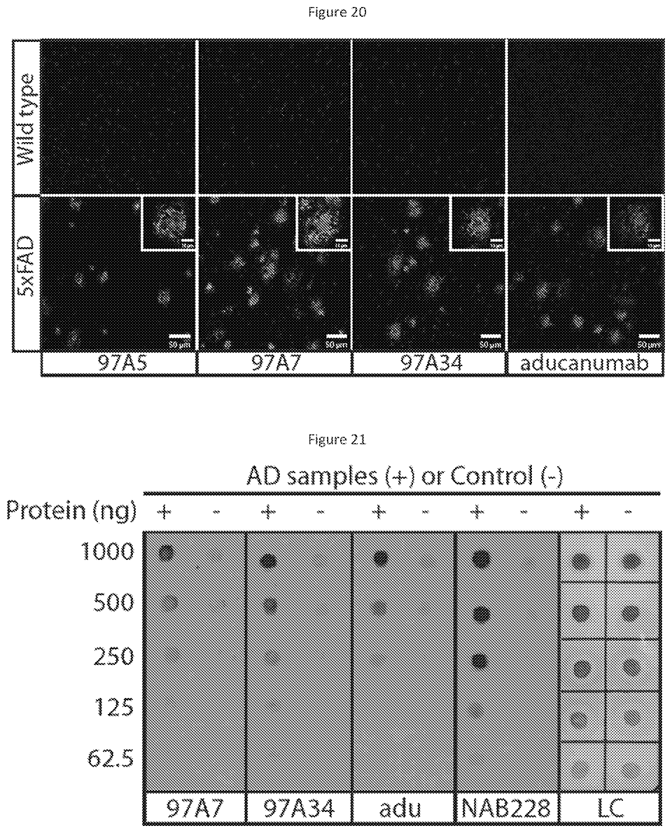

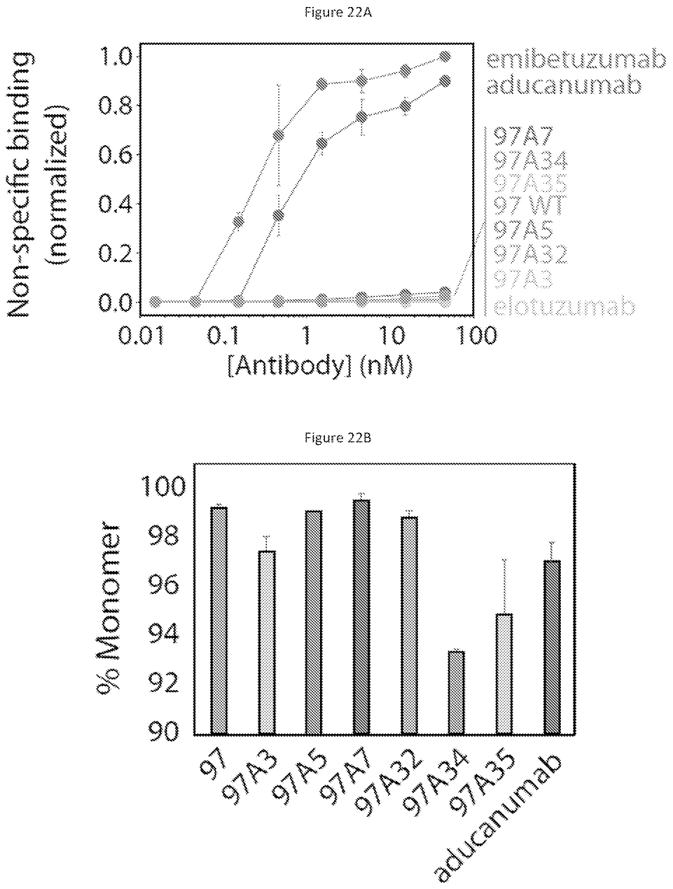

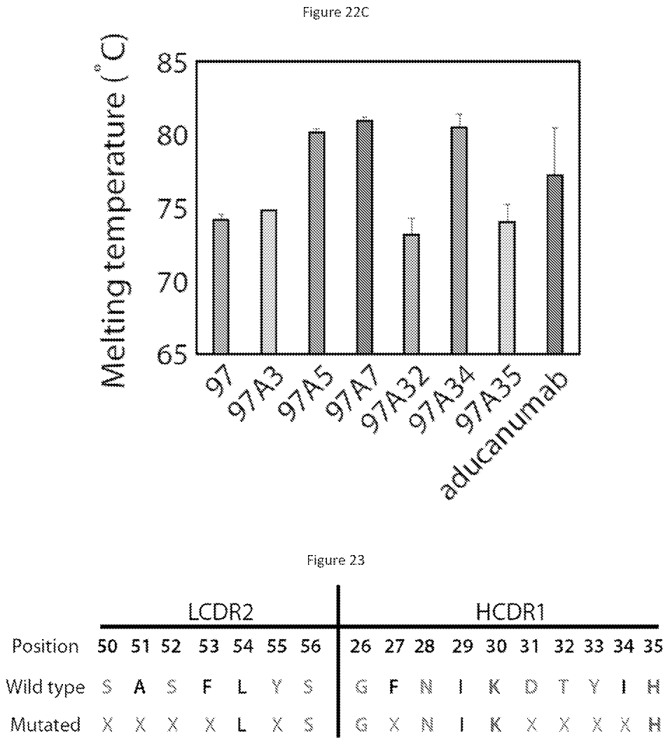

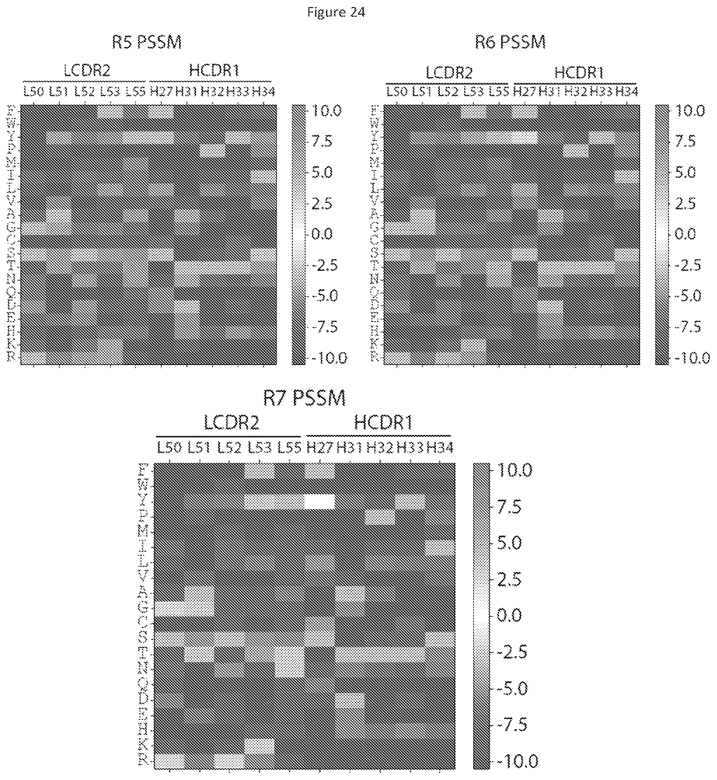

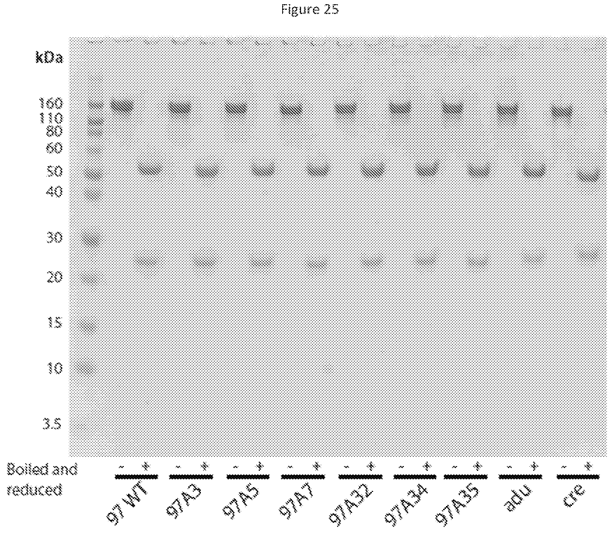

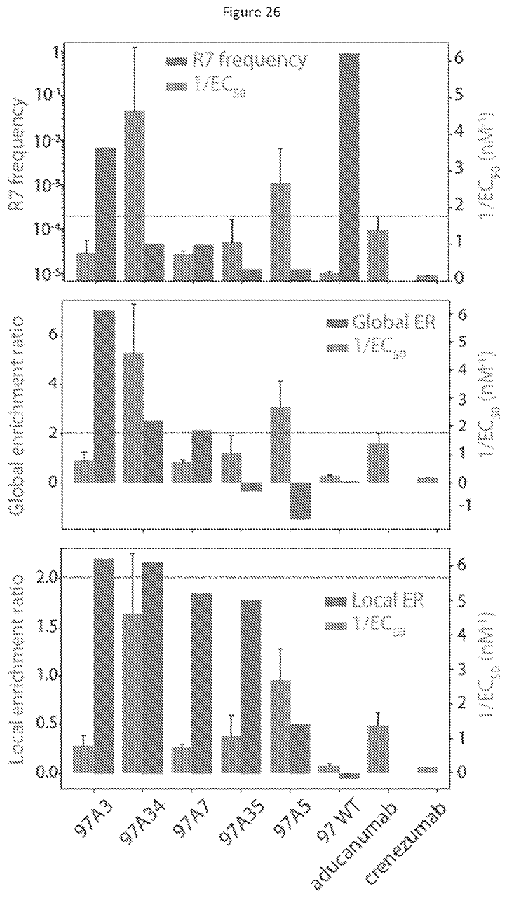

MACS selections against Ab42 fibrils yielded a single enriched antibody variant with five mutations in light chain CDR2 (denoted “97A3”; FIGS. 10A-10C). Notably, this clone displayed higher apparent affinity than the parental antibody (˜6-fold improvement) and aducanumab (˜3-fold improvement; FIG. 10A). Given that different batches of fibrils were used to perform the binding experiments in FIGS. 5 and 10, the EC₅₀ values for clone 97 (8±1 nM in FIGS. 5 and 18±1 nM in FIG. 10) and aducanumab (3±1 nM in FIGS. 5 and 10±2 nM in FIG. 10) were modestly different. Moreover, the affinity-matured antibody 97A3 displayed high conformational specificity similar to clone 97 and aducanumab (FIG. 10B) and low non-specific binding that was similar to clone 97 and much lower than aducanumab (FIG. 10C). Moreover, 97A3 was mostly monomeric after one-step Protein A purification (>>93%) and displayed high stability (Tₘ

of 69±0.5° C.) that was similar to the parental antibody (97% monomer and Tₘ of 68±2° C.; FIGS. 11A-11B).

These results demonstrate that the affinity maturation methods described herein can be used to generate antibodies with superior affinity and levels of non-specific binding relative to aducanumab while maintaining high conformational specificity and thermal stability.

The present disclosure provides a rational and systematic approach for affinity maturing conformational antibodies specific insoluble polypeptide aggregates. Indeed, a surprisingly high level of success at identifying affinity-matured clones was achieved using the methods described herein. For example, all 19 of the clones that were identified via the deep sequencing analysis displayed increased affinity during primary screens performed with immunodot blots. Moreover, all of the 15 clones tested for conformational specificity displayed low binding to disaggregated Aβ in our competition experiments (FIGS. 5C and 5D). Finally, all of 15 clones tested for Aβ fibrils affinity displayed 8- to 20-fold improvements in their EC₅₀ values (FIGS. 5A and 5B).

The success of this approach may be related to how sets of mutations were identified that most correlated with improved enrichment ratios using deep sequencing. This process assumes that the sets of mutations (e.g., sets of six mutations) govern the improved behavior and ignores the residues at the other randomized sites. Introducing these sets of mutations into the parental antibody, without introducing any mutations at the other sites, may improve antibody affinity. However, this approach was much less robust, as <50% (7 out of 15) of antibody mutants tested using this strategy showed increased affinity (as judged by immunoblots; data not shown). This suggests that mutated residues at sites not considered in a given mutational set (e.g., sites 1 and 2 when evaluating sets of mutations at sites 3-8) contribute to the overall binding activity and were important to the success of the affinity-maturation strategy described herein.

The strategy for designing sub-libraries with particular types of mutations disclosed herein also may have contributed to the success of selecting antibody variants with improved affinity while maintaining both conformational specificity for Aβ aggregates and low levels of off-target binding. Given the acidic nature of Aβ42 (theoretical pI of 5.3), it is common to select positively-charged mutations that increase antibody affinity due to attractive electrostatic interactions (13,23). However, over-enrichment in positively-charged residues in antibody CDRs is a key risk factor for off-target binding (12,15,16,24). Therefore, positively-charged mutations were eliminated from the library design, which may have reduced (at least partially) the strong avidity effects due to reduction of relatively long range, attractive electrostatic interactions during library sorting.

Sequences

| SEQ ID NO: | Sequence Name | Sequence |
|---|---|---|
| 1 | AF1 HCDR1 | SGFNIKDTYIH |
| 2 | AF1 HCDR2 | RIYPTNGYTRYADSVKG |
| 3 | AF1 HCDR3 | ARDGYDGSYFVGYDYNDFYDY |
| 4 | AF1 LCDR1 | RASQDVNTAVA |
| 5 | AF1 LCDR2 | SASFLYS |
| 6 | AF1 LCDR3 | QQHYTTPPT |
| 7 | Clone 88 VH | EVQLVESGGGLVQPGGSLRLSCAASGFNIKDTYIHWVRQAPGKGLEWVARIYPN |
| | | SGATRYAGSVKGRFTISADTSKNTAYLQMNSLRAEDTAVYYCARDGYDGSYFV |
| | | GYDYNDFYDYWGQGTLVTVSS |
| 8 | Clone 88 VL | DIQMTQSPSSLSASVGDRVTITCRASQNVYAAVTWYQQKPGKAPKLLIYSASFL |
| | | YSGVPSRFSGSRSGTDFTLTISSLQPEDFATYYCQQHSTYPPTFGQGTKVEIK |
| 9 | Clone 89 VH | EVQLVESGGGLVQPGGSLRLSCAASGFNIKDTYIHWVRQAPGKGLEWVARIYPN |
| | | SGATRYAGSVKGRFTISADTSKNTAYLQMNSLRAEDTAVYYCARDGYDGSYFV |
| | | GYDYNDFYDYWGQGTLVTVSS |
| 10 | Clone 89 VL | DIQMTQSPSSLSASVGDRVTITCRASQNVYSAVSWYQQKPGKAPKLLIYSASFLY |
| | | SGVPSRFSGSRSGTDFTLTISSLQPEDFATYYCQQHYTYPPTFGQGTKVEIK |
| 11 | Clone 91 VH | EVQLVESGGGLVQPGGSLRLSCAASGFNIKDTYIHWVRQAPGKGLEWVARIYPA |
| | | SGTTRYAGSVKGRFTISADTSKNTAYLQMNSLRAEDTAVYYCARDGYDGSYFV |
| | | GYDYNDFYDYWGQGTLVTVSS |
| 12 | Clone 91 VL | DIQMTQSPSSLSASVGDRVTITCRASQNVYSAVSWYQQKPGKAPKLLIYSASFLY |
| | | SGVPSRFSGSRSGTDFTLTISSLQPEDFATYYCQQHSTYPPTFGQGTKVEIK |
| 13 | Clone 92 VH | EVQLVESGGGLVQPGGSLRLSCAASGFNIKDTYIHWVRQAPGKGLEWVARIYPA |
| | | SGTTRYAASVKGRFTISADTSKNTAYLQMNSLRAEDTAVYYCARDGYDGSYFV |
| | | GYDYNDFYDYWGQGTLVTVSS |
| 14 | Clone 92 VL | DIQMTQSPSSLSASVGDRVTITCRASQNVYSAVSWYQQKPGKAPKLLIYSASFLY |
| | | SGVPSRFSGSRSGTDFTLTISSLQPEDFATYYCQQHSTYPPTFGQGTKVEIK |
| 15 | Clone 93 VH | EVQLVESGGGLVQPGGSLRLSCAASGFNIKDTYIHWVRQAPGKGLEWVARIYPS |
| | | NGYTRYAGSVKGRFTISADTSKNTAYLQMNSLRAEDTAVYYCARDGYDGSYFV |
| | | GYDYNDFYDYWGQGTLVTVSS |
| 16 | Clone 93 VL | DIQMTQSPSSLSASVGDRVTITCRASQNVYNAVTWYQQKPGKAPKLLIYSASFL |
| | | YSGVPSRFSGSRSGTDFTLTISSLQPEDFATYYCQQHNTYPPTFGQGTKVEIK |
| 17 | Clone 95 VH | EVQLVESGGGLVQPGGSLRLSCAASGFNIKDTYIHWVRQAPGKGLEWVARIYPA |
| | | SGSTRYAASVKGRFTISADTSKNTAYLQMNSLRAEDTAVYYCARDGYDGSYFV |
| | | GYDYNDFYDYWGQGTLVTVSS |
| 18 | Clone 95 VL | DIQMTQSPSSLSASVGDRVTITCRASQNVYSAVTWYQQKPGKAPKLLIYSASFLY |
| | | SGVPSRFSGSRSGTDFTLTISSLQPEDFATYYCQQHSTSPPTFGQGTKVEIK |
| 19 | Clones 97, 97A3, 97A6 | EVQLVESGGGLVQPGGSLRLSCAASGFNIKDTYIHWVRQAPGKGLEWVARIYPA |
| | VH | SGATRYADSVKGRFTISADTSKNTAYLQMNSLRAEDTAVYYCARDGYDGSYFV |
| | | GYDYNDFYDYWGQGTLVTVSS |
| 20 | Clones 97, 97A32, | DIQMTQSPSSLSASVGDRVTITCRASQNVAYAVTWYQQKPGKAPKLLIYSASFL |
| | 97A34, 97A35 VL | YSGVPSRFSGSRSGTDFTLTISSLQPEDFATYYCQQHSTYPPTFGQGTKVEIK |
| 21 | Clone 98 VH | EVQLVESGGGLVQPGGSLRLSCAASGFNIKDTYIHWVRQAPGKGLEWVARIYPS |
| | | NGYTRYAASVKGRFTISADTSKNTAYLQMNSLRAEDTAVYYCARDGYDGSYFV |
| | | GYDYNDFYDYWGQGTLVTVSS |
| 22 | Clone 98 VL | DIQMTQSPSSLSASVGDRVTITCRASQNVAYAVSWYQQKPGKAPKLLIYSASFLY |
| | | SGVPSRFSGSRSGTDFTLTISSLQPEDFATYYCQQHSTYPPTFGQGTKVEIK |
| 23 | Clone 101 VH | EVQLVESGGGLVQPGGSLRLSCAASGFNIKDTYIHWVRQAPGKGLEWVARIYPA |
| | | SGSTRYAGSVKGRFTISADTSKNTAYLQMNSLRAEDTAVYYCARDGYDGSYFV |
| | | GYDYNDFYDYWGQGTLVTVSS |
| 24 | Clone 101 VL | DIQMTQSPSSLSASVGDRVTITCRASQNVYSAVTWYQQKPGKAPKLLIYSASFLY |
| | | SGVPSRFSGSRSGTDFTLTISSLQPEDFATYYCQQHSTYPPTFGQGTKVEIK |
| 25 | Clone 102 VH | EVQLVESGGGLVQPGGSLRLSCAASGFNIKDTYIHWVRQAPGKGLEWVARIYPA |
| | | SGYTRYAASVKGRFTISADTSKNTAYLQMNSLRAEDTAVYYCARDGYDGSYFV |
| | | GYDYNDFYDYWGQGTLVTVSS |
| 26 | Clone 102 VL | DIQMTQSPSSLSASVGDRVTITCRASQNVASAVTWYQQKPGKAPKLLIYSASFLY |
| | | SGVPSRFSGSRSGTDFTLTISSLQPEDFATYYCQQHYTYPPTFGQGTKVEIK |
| 27 | Clone 97A3 VL | DIQMTQSPSSLSASVGDRVTITCRASQNVAYAVTWYQQKPGKAPKLLIYGTRYL |
| | | NSGVPSRFSGSRSGTDFTLTISSLQPEDFATYYCQQHSTYPPTFGQGTKVEIK |
| 28 | Clones 88, 89 HCDR2 | RIYPNSGATRYAGSVKG |
| 29 | Clone 91 HCDR2 | RIYPASGTTRYAGSVKG |
| 30 | Clone 92 HCDR2 | RIYPASGTTRYAASVKG |
| 31 | Clone 93 HCDR2 | RIYPSNGYTRYAGSVKG |
| 32 | Clone 95 HCDR2 | RIYPASGSTRYAASVKG |
| 33 | Clones 97 and 97A3, | RIYPASGATRYADSVKG |
| | 97A5, 97A7, 97A32, | |
| | 97A34, 97A35 HCDR2 | |
| 34 | Clone 98 HCDR2 | RIYPSNGYTRYAASVKG |
| 35 | Clone 101 HCDR2 | RIYPASGSTRYAGSVKG |
| 36 | Clone 102 HCDR2 | RIYPASGYTRYAASVKG |
| 37 | Clone 88 LCDR1 | RASQNVYAAVT |
| 38 | Clones 89, 91, 92 | RASQNVYSAVS |
| | LCDR1 | |
| 39 | Clone 93 LCDR1 | RASQNVYNAVT |
| 40 | Clones 95, 101 LCDR1 | RASQNVYSAVT |
| 41 | Clones 97, 97A3, 97A5, | RASQNVAYAVT |
| | 97A6, 97A7, 97A32, | |
| | 97A34. 97A35 LCDR1 | |
| 42 | Clone 98 LCDR1 | RASQNVAYAVS |
| 43 | Clone 102 LCDR1 | RASQNVASAVT |
| 44 | Clones 88, 91, 92, 97, | QQHSTYPPT |
| | 97A3, 97A5, 97A6, | |
| | 97A7, 97A32, 97A34, | |
| | 97a35, 98, 101 LCDR3 | |
| 45 | Clones 89, 102 LCDR3 | QQHYTYPPT |
| 46 | Clone 93 LCDR3 | QQHNTYPPT |
| 47 | Clone 95 LCDR3 | QQHSTSPPT |
| 48 | Clone 97A6 VL | DIQMTQSPSSLSASVGDRVTITCRASQNVAYAVTWYQQKPGKAPKLLIYSYNKL |
| | | ASGVPSRFSGSRSGTDFTLTISSLQPEDFATYYCQQHSTYPPTFGQGTKVEIK |
| 49 | Clone 97A5, 97A7 VH | EVQLVESGGGLVQPGGSLRLSCAASGYNIKDTYIHWVRQAPGKGLEWVARIYP |
| | | ASGATRYADSVKGRFTISADTSKNTAYLQMNSLRAEDTAVYYCARDGYDGSYF |
| | | VGYDYNDFYDYWGQGTLVTVSS |
| 50 | Clone 97A7 VL | DIQMTQSPSSLSASVGDRVTITCRASQNVAYAVTWYQQKPGKAPKLLIYSAIFLY |
| | | SGVPSRFSGSRSGTDFTLTISSLQPEDFATYYCQQHSTYPPTFGQGTKVEIK |
| 51 | Clone 97A5 VL | DIQMTQSPSSLSASVGDRVTITCRASQNVAYAVTWYQQKPGKAPKLLIYSSSFLY |
| | | SGVPSRFSGSRSGTDFTLTISSLQPEDFATYYCQQHSTYPPTFGQGTKVEIK |
| 52 | Clones 97A34, 97A35 | EVQLVESGGGLVQPGGSLRLSCAASGYNIKATYIHWVRQAPGKGLEWVARIYP |
| | VH | ASGATRYADSVKGRFTISADTSKNTAYLQMNSLRAEDTAVYYCARDGYDGSYF |
| | | VGYDYNDFYDYWGQGTLVTVSS |
| 53 | Clone 97A32 VH | EVQLVESGGGLVQPGGSLRLSCAASGFNIKGTYSHWVRQAPGKGLEWVARIYP |
| | | ASGATRYADSVKGRFTISADTSKNTAYLQMNSLRAEDTAVYYCARDGYDGSYF |
| | | VGYDYNDFYDYWGQGTLVTVSS |
| 54 | Clones 97A34 and | SGYNIKATYIH |
| | 97A35 HCDR1 | |
| 55 | Clone 97A32 HCDR1 | SGFNIKGTYSH |
| 56 | Clones 97A5 and 97A7 | SGYNIKDTYIH |
| | HCDR1 | |
| 57 | Clone 97 Heavy Chain | EVQLVESGGGLVQPGGSLRLSCAASGFNIKDTYIHWVRQAPGKGLEWVARIYPA |
| | | SGATRYADSVKGRFTISADTSKNTAYLQMNSLRAEDTAVYYCARDGYDGSYFV |
| | | GYDYNDFYDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRWQQGNV |
| | | FSCSVMHEALHNHYTQKSLSLSPGK |
| 58 | Clone 97 Light Chain | DIQMTQSPSSLSASVGDRVTITCRASQNVAYAVTWYQQKPGKAPKLLIYSASFL |
| | | YSGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 59 | Clone 97A34 Heavy | EVQLVESGGGLVQPGGSLRLSCAASGYNIKATYIHWVRQAPGKGLEWVARTYP |
| | Chain | ASGATRYADSVKGRFTISADTSKNTAYLQMNSLRAEDTAVYYCARDGYDGSYF |
| | | VGYDYNDFYDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGK |
| 60 | Clone 97A34 Light | DIQMTQSPSSLSASVGDRVTITCRASQNVAYAVTWYQQKPGKAPKLLIYSASFL |
| | Chain | YSGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 61 | Clone 97A7 Heavy | EVQLVESGGGLVQPGGSLRLSCAASGYNIKDTYIHWVRQAPGKGLEWVARIYP |
| | Chain | ASGATRYADSVKGRFTISADTSKNTAYLQMNSLRAEDTAVYYCARDGYDGSYF |
| | | VGYDYNDFYDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGK |
| 62 | Clone 97A7 Light | DIQMTQSPSSLSASVGDRVTITCRASQNVAYAVTWYQQKPGKAPKLLIYSAIFLY |
| | Chain | SGVPSRFSGSRSGTDFTLTISSLQPEDFATYYCQQHSTYPPTFGQGTKVEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 63 | Clone 97A5 Heavy | EVQLVESGGGLVQPGGSLRLSCAASGYNIKDTYIHWVRQAPGKGLEWVARIYP |
| | Chain | ASGATRYADSVKGRFTISADTSKNTAYLQMNSLRAEDTAVYYCARDGYDGSYF |
| | | VGYDYNDFYDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGK |
| 64 | Clone 97A5 Light | DIQMTQSPSSLSASVGDRVTITCRASQNVAYAVTWYQQKPGKAPKLLIYSSSFLY |
| | Chain | SGVPSRFSGSRSGTDFTLTISSLQPEDFATYYCQQHSTYPPTFGQGTKVEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 65 | Clone 97A32 Heavy | EVQLVESGGGLVQPGGSLRLSCAASGFNIKGTYSHWVRQAPGKGLEWVARIYP |
| | Chain | ASGATRYADSVKGRFTISADTSKNTAYLQMNSLRAEDTAVYYCARDGYDGSYF |
| | | VGYDYNDFYDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGK |
| 66 | Clone 97A32 Light | DIQMTQSPSSLSASVGDRVTITCRASQNVAYAVTWYQQKPGKAPKLLIYSASFL |
| | Chain | YSGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 67 | Clone 97A3 Heavy | EVQLVESGGGLVQPGGSLRLSCAASGFNIKDTYIHWVRQAPGKGLEWVARIYPA |
| | Chain | SGATRYADSVKGRFTISADTSKNTAYLQMNSLRAEDTAVYYCARDGYDGSYFV |
| | | GYDYNDFYDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRWQQGNV |
| | | FSCSVMHEALHNHYTQKSLSLSPGK |
| 68 | Clone 97A3 Light | DIQMTQSPSSLSASVGDRVTITCRASQNVAYAVTWYQQKPGKAPKLLIYGTRYL |
| | Chain | NSGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 69 | Clone 97A35 Heavy | EVQLVESGGGLVQPGGSLRLSCAASGYNIKATYIHWVRQAPGKGLEWVARIYP |
| | Chain | ASGATRYADSVKGRFTISADTSKNTAYLQMNSLRAEDTAVYYCARDGYDGSYF |
| | | VGYDYNDFYDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLIVLHQ |
| | | DWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGK |
| 70 | Clone 97A35 Light | DIQMTQSPSSLSASVGDRVTITCRASQNVAYAVTWYQQKPGKAPKLLIYSASFL |
| | Chain | YSGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 71 | Clone 97A6 Heavy | EVQLVESGGGLVQPGGSLRLSCAASGFNIKDTYIHWVRQAPGKGLEWVARIYPA |
| | Chain | SGATRYADSVKGRFTISADTSKNTAYLQMNSLRAEDTAVYYCARDGYDGSYFV |
| | | GYDYNDFYDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRWQQGNV |
| | | FSCSVMHEALHNHYTQKSLSLSPGK |
| 72 | Clone 97A6 Light | DIQMTQSPSSLSASVGDRVTITCRASQNVAYAVTWYQQKPGKAPKLLIYSYNKL |
| | Chain | ASGVPSRFSGSRSGTDFTLTISSLQPEDFATYYCQQHSTYPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |

### REFERENCES

All references, including publications, patent applications, and patents, cited herein are hereby incorporated by reference to the same extent as if each reference were individually and specifically indicated to be incorporated by reference and were set forth in its entirety herein.

1. Chiti. F., and Dobson, C. M. (2017) Protein Misfolding, Amyloid Formation, and Human Disease: A Summary of Progress Over the Last Decade. Annu Rev Biochem 86, 27-68
2. Ross, C. A., and Poirier, M. A. (2004) Protein aggregation and neurodegenerative disease. Nat Med 10, S10-17
3. Glabe, C. G. (2004) Conformation-dependent antibodies target diseases of protein misfolding. Trends Biochem Sci 29, 542-547
4. Droste, P., Frenzel, A., Steinwand, M., Pelat, T., Thullier, P., Hust, M., Lashuel, H., and Dubel, S. (2015) Structural differences of amyloid-beta fibrils revealed by antibodies from phage display. BMC Biotechnol 15, 57
5. Hatami, A., Albay, R., 3rd, Monjazeb, S., Milton, S., and Glabe, C. (2014) Monoclonal antibodies against Abeta42 fibrils distinguish multiple aggregation state polymorphisms in vitro and in Alzheimer disease brain. J Biol Chem 289, 32131-32143
6. Kayed. R., Head, E., Thompson, J. L., McIntire, T. M., Milton, S. C., Cotman, C. W., and Glabe, C. G. (2003) Common Structure of Soluble Amyloid Oligomers Implies Common Mechanism of Pathogenesis. Science 300, 486-489
7. Kanaan, N. M., Cox, K., Alvarez, V. E., Stein, T. D., Poncil, S., and McKee, A. C. (2016) Characterization of Early Pathological Tau Conformations and Phosphorylation in Chronic Traumatic Encephalopathy. J Neuropathol Exp Neurol 75, 19-34
8. Bonito-Oliva, A., Schedin-Weiss, S., Younesi, S. S., Tiiman, A., Adura, C., Paknejad, N., Brendel, M., Romin, Y., Parchem, R. J., Graff, C., Vukojevic, V., Tjernberg, L. O., Terenius, L., Winblad, B., Sakmar, T. P., and Graham, W. V. (2019) Conformation-specific antibodies against multiple amyloid protofibril species from a single amyloid immunogen. J Cell Mol Med 23, 2103-2114
9. Combs, B., Hamel, C., and Kanaan, N. M. (2016) Pathological conformations involving the amino terminus of tau occur early in Alzheimer's disease and are differentially detected by monoclonal antibodies. Neurobiol Dis 94, 18-31
10. Kanaan, N. M., Morfini, G. A., LaPointe, N. E., Pigino, G. F., Patterson, K. R., Song, Y., Andreadis, A., Fu, Y., Brady, S. T., and Binder, L. I. (2011) Pathogenic forms of tau inhibit kincsin-dependent axonal transport through a mechanism involving activation of axonal phosphotransferases. J Neurosci 31, 9858-9868
11. Bodani, R. U., Sengupta, U., Castillo-Carranza, D. L., Guerrero-Munoz. M. J., Gerson. J. E., Rudra, J., and Kayed, R. (2015) Antibody against Small Aggregated Peptide Specifically Recognizes Toxic Abeta-42 Oligomers in Alzheimer's Disease. ACS Chem Neurosci 6, 1981-1989
12. Lasagna-Reeves, C. A., Castillo-Carranza, D. L., Sengupta, U., Sarmiento, J., Troncoso, J., Jackson. G. R., and Kayed, R. (2012) Identification of oligomers at early stages of tau aggregation in Alzheimer's disease. FASEB J 26, 1946-1959
13. Weihofen, A., Liu, Y., Arndt, J. W., Huy, C., Quan, C., Smith, B. A., Baeriswyl, J. L., Cavegn, N., Senn, L., Su, L., Marsh, G., Auluck, P. K., Montrasio, F., Nitsch, R. M., Hirst, W. D., Cedarbaum, J. M., Pepinsky, R. B., Grimm. J., and Weinreb, P. H. (2019) Development of an aggregate-selective, human-derived alpha-synuclein antibody BIIB054 that ameliorates disease phenotypes in Parkinson's disease models. Neurobiol Dis 124, 276-288
14. Arndt, J. W., Qian, F., Smith, B. A., Quan, C., Kilambi, K. P., Bush, M. W., Walz, T., Pepinsky, R. B., Bussiere. T., Hamann, S., Cameron, T. O., and Weinreb, P. H. (2018) Structural and kinetic basis for the selectivity of aducanumab for aggregated forms of amyloid-beta. Sci Rep 8, 6412
15. Sevigny, J., Chiao, P., Bussiere, T., Weinreb, P. H., Williams, L., Maier, M., Dunstan, R., Salloway, S., Chen, T., Ling, Y., O'Gorman, J., Qian, F., Arastu, M., Li. M., Chollate, S., Brennan, M. S., Quintero-Monzon, O., Scannevin, R. H., Arnold, H. M., Engber, T., Rhodes, K., Ferrero, J., Hang, Y., Mikulskis. A., Grimm, J., Hock, C., Nitsch, R. M., and Sandrock, A. (2016). The antibody aducanumab reduces Abeta plaques in Alzheimer's disease. Nature 537, 50-56
16. Castillo-Carranza, D. L., Sengupta, U., Guerrero-Munoz, M. J., Lasagna-Reeves, C. A., Gerson, J. E., Singh, G., Estes, D. M., Barrett, A. D., Dineley, K. T., Jackson, G. R., and Kayed, R. (2014) Passive immunization with Tau oligomer monoclonal antibody reverses tauopathy phenotypes without affecting hyperphosphorylated neurofibrillary tangles. J Neurosci 34, 4260-4272
17. Gong, Y., Chang. L., Viola, K. L., Lacor, P. N., Lambert, M. P., Finch, C. E., Krafft, G. A., and Klein, W. L. (2003) Alzheimer's disease-affected brain: presence of oligomeric A beta ligands (ADDLs) suggests a molecular basis for reversible memory loss. Proc Natl Acad Sci U S A 100, 10417-10422
18. Lambert, M. P., Velasco, P. T., Chang. L., Viola, K. L., Fernandez, S., Lacor, P. N., Khuon, D., Gong, Y., Bigio, E. H., Shaw, P., De Felice, F. G., Krafft, G. A., and Klein, W. L. (2007) Monoclonal antibodies that target pathological assemblies of Abeta. J Neurochem 100, 23-35
19. Lambert, M. P., Viola, K. L., Chromy, B. A., Chang, L., Morgan, T. E., Yu. J., Venton, D. L., Krafft, G. A., Finch, C. E., and Klein. W. L. (2001) Vaccination with soluble Aβ oligomers generates toxicity-neutralizing antibodies. Journal of Neurochemistry 79, 595-605
20. Kayed, R., Head, E., Sarsoza, F., Saing, T., Cotman, C. W., Necula, M., Margol, L., Wu, J., Breydo, L., Thompson, J. L., Rasool, S., Gurlo, T., Butler, P., and Glabe, C. G. (2007) Fibril specific, conformation dependent antibodies recognize a generic epitope common to amyloid fibrils and fibrillar oligomers that is absent in prefibrillar oligomers. Mol Neurodegener 2, 18
21. Gibbons, G. S., Kim, S. J., Robinson, J. L., Changolkar, L., Irwin, D. J., Shaw, L. M., Lee, V. M., and Trojanowski, J. Q. (2019) Detection of Alzheimer's disease (AD) specific tau pathology with conformation-selective anti-tau monoclonal antibody in co-morbid frontotemporal lobar degeneration-tau (FTLD-tau). Acta Neuropathol Commun 7, 34
22. Covell, D. J., Robinson, J. L., Akhtar, R. S., Grossman, M., Weintraub, D., Bucklin, H. M., Pitkin, R. M., Riddle, D., Yousef, A., Trojanowski, J. Q., and Lee, V. M. (2017) Novel conformation-selective alpha-synuclein antibodies raised against different in vitro fibril forms show distinct patterns of Lewy pathology in Parkinson's disease. Neuropathol Appl Neurobiol 43, 604-620
23. Julian, M. C., Rabia, L. A., Desai, A. A., Arsiwala, A., Gerson, J. E., Paulson, H. L., Kane, R. S., and Tessier, P. M. (2019) Nature-inspired design and evolution of anti-amyloid antibodies. J Biol Chem 294, 8438-8451
24. Stimple. S. D., Kalyoncu, S., Desai, A. A., Mogensen, J. E., Spang, L. T., Asgreen, D. J., Staby, A., and Tessier, P. M. (2019) Sensitive detection of glucagon aggregation using amyloid fibrilspecific antibodies. Biotechnol Bioeng 116, 1868-1877
25. Munke, A., Persson, J., Weiffert, T., De Genst, E., Meisl, G., Arosio, P., Carnerup, A., Dobson, C. M., Vondruscolo. M., Knowles, T. P. J., and Linse, S. (2017) Phage display and kinetic selection of antibodies that specifically inhibit amyloid self-replication. Proceedings of the National Academy of Sciences 114. 6444-6449
26. Sebollela, A., Cline, E. N., Popova, I., Luo, K., Sun, X., Ahn, J., Barcelos, M. A., Bezerra, V. N., Lyra, E. S. N. M., Patel, J., Pinheiro, N. R., Qin, L. A., Kamel, J. M., Weng, A., DiNunno, N., Bebenek. A. M., Velasco. P. T., Viola, K. L., Lacor, P. N., Ferreira, S. T., and Klein, W. L. (2017) A human scFv antibody that targets and neutralizes high molecular weight pathogenic amyloid-beta oligomers. J Neurochem 142, 934-947
27. Lec, C. C., Julian, M. C., Tiller, K. E., Meng, F., DuConge, S. E., Akter, R., Raleigh, D. P., and Tessier, P. M. (2016) Design and Optimization of Anti-amyloid Domain Antibodies Specific for beta-Amyloid and Islet Amyloid Polypeptide. J Biol Chem 291. 2858-2873
28. Silverman, J. M., Gibbs, E., Peng, X., Martens, K. M., Balducci, C., Wang, J., Yousefi, M., Cowan, C. M., Lamour, G., Louadi, S., Ban, Y., Robert, J., Stukas, S., Forloni, G., Hsiung, G. R., Plotkin, S. S., Wellington, C. L., and Cashman, N. R. (2018) A Rational Structured Epitope Defines a Distinct Subclass of Toxic Amyloid-beta Oligomers. ACS Chem Neurosci 9, 1591-1606
29. Gibbs. E., Silverman, J. M., Zhao, B., Peng, X., Wang, J., Wellington, C. L., Mackenzie. I. R., Plotkin, S. S., Kaplan, J. M., and Cashman, N. R. (2019) A Rationally Designed Humanized Antibody Selective for Amyloid Beta Oligomers in Alzheimer's Disease. Sci Rep 9, 9870
30. Perchiacca, J. M., Ladiwala, A. R., Bhattacharya, M., and Tessier, P. M. (2012) Structure-based design of conformation- and sequence-specific antibodies against amyloid beta. Proc Natl Acad Sci U S A 109, 84-89
31. Aprile, F. A., Sormanni, P., Podpolny, M., Chhangur, S., Needham, L. M., Ruggeri, F. S., Perni, M., Limbocker, R., Heller, G. T., Sneideris, T., Scheidt, T., Mannini, B., Habchi, J., Lee, S. F., Salinas, P. C., Knowles, T. P. J., Dobson, C. M., and Vendruscolo, M. (2020) Rational design of a conformation-specific antibody for the quantification of Abeta oligomers. Proc Natl Acad Sci U S A, ePub June 3
32. Kulenkampff, K., Aprile, F. A., Sormanni, P., Ranasinghe, R. T., Klenerman, D., and Vendruscolo, M. (2020) Rational Design of Conformation-Specific Antibodies for Tau Oligomers. Biophysical Journal 118, 370a-371a
33. Vaikath, N. N., Majbour, N. K., Paleologou, K. E., Ardah, M. T., van Dam. E., van de Berg, W. D., Forrest, S. L., Parkkinen, L., Gai, W. P., Hattori, N., Takanashi, M., Lee, S. J., Mann, D. M., Imai, Y., Halliday, G. M., Li, J. Y., and El-Agnaf, O. M. (2015) Generation and characterization of novel conformation-specific monoclonal antibodies for alpha-synuclein pathology. Neurobiol Dis 79. 81-99
34. Gibbons. G. S., Banks, R. A., Kim, B., Changolkar, L., Riddle. D. M., Leight, S. N., Irwin, D. J., Trojanowski, J. Q., and Lee, V. M. Y. (2018) Detection of Alzheimer Disease (AD)-Specific Tau Pathology in AD and NonAD Tauopathies by Immunohistochemistry With Novel Conformation-Selective Tau Antibodies. J Neuropathol Exp Neurol 77, 216-228
35. Tiller, K. E., Chowdhury, R., Li. T., Ludwig, S. D., Sen. S., Maranas, C. D., and Tessier, P. M. (2017) Facile Affinity Maturation of Antibody Variable Domains Using Natural Diversity Mutagenesis. Front Immunol 8, 986
36. Jain, T., Sun, T., Durand, S., Hall, A., Houston, N. R., Nett, J. H., Sharkey, B., Bobrowicz, B., Caffry, I., Yu, Y., Cao, Y., Lynaugh, H., Brown, M., Baruah, H., Gray, L. T., Krauland, E. M., Xu, Y., Vasquez, M., and Wittrup, K. D. (2017) Biophysical properties of the clinical-stage antibody landscape. Proc Natl Acad Sci U S A 114, 944-949
37. Swindells, M. B., Porter, C. T., Couch, M., Hurst, J., Abhinandan, K. R., Nielsen, J. H., Macindoc, G., Hetherington, J., and Martin, A. C. (2017) abYsis: Integrated Antibody Sequence and Structure-Management. Analysis, and Prediction. J Mol Biol 429, 356-364
38. Rabia, L. A., Zhang, Y., Ludwig, S. D., Julian, M. C., and Tessier, P. M. (2018) Net charge of antibody complementarity-determining regions is a key predictor of specificity. Protein Eng Des Sel 31, 409-418
39. Fellouse. F. A., Wiesmann, C., and Sidhu, S. S. (2004) Synthetic antibodies from a four-aminoacid code: a dominant role for tyrosine in antigen recognition. Proc Natl Acad Sci U S A 101. 12467-12472
40. Tiller, K. E., Li, L., Kumar, S., Julian, M. C., Garde, S., and Tessier, P. M. (2017) Arginine mutations in antibody complementarity-determining regions display context-dependent affinity/specificity trade-offs. J Biol Chem 292. 16638-16652
41. Zhang, Y., Wu, L., Gupta, P., Desai, A. A., Smith, M. D., Rabia, L. A., Ludwig, S. D., and Tessier, P. M. (2020) Physicochemical rules for identifying monoclonal antibodies with drug-like specificity. Mol Pharm, ePub May 26
42. Datta-Mannan, A., Thangaraju, A., Leung, D., Tang, Y., Witcher, D. R., Lu, J., and Wroblewski, V. J. (2015) Balancing charge in the complementarity-determining regions of humanized mAbs without affecting pI reduces non-specific binding and improves the pharmacokinetics.MAbs 7, 483-493
43. Wardemann, H., Yurasov, S., Schaefer, A., Young, J. W., Meffre, E., and Nussenzweig, M. C. (2003) Predominant Autoantibody Production by Early Human B Cell Precursors. Science 301, 1374-1377
44. Hong, G., Chappey, O., Niel, E., and Scherrmann, J. M. (2000) Enhanced cellular uptake and transport of polyclonal immunoglobulin G and fab after their cationization. J Drug Target 8, 67-77
45. Datta-Mannan, A., Lu, J., Witcher. D. R., Leung, D., Tang, Y., and Wroblewski, V. J. (2015) The interplay of non-specific binding, target-mediated clearance and FcRn interactions on the pharmacokinetics of humanized antibodies. MAbs 7, 1084-1093
46. Birtalan, S., Fisher, R. D., and Sidhu. S. S. (2010) The functional capacity of the natural amino acids for molecular recognition. Mol Biosyst 6, 1186-1194
47. Arndt, J. W., Qian, F., Smith, B. A., Quan, C., Kilambi, K. P., Bush, M. W., Walz, T., Pepinsky, R. B., Bussière, T., Hamann, S., Cameron, T. O., and Weinreb, P. H. (2018) Structural and kinetic basis for the selectivity of aducanumab for aggregated forms of amyloid-β. Sci Rep 8, 6412
48. van Dyck, C. H. (2018) Anti-Amyloid-beta Monoclonal Antibodies for Alzheimer's Disease: Pitfalls and Promise. Biol Psychiatry 83, 311-319
49. Spencer, S., Bethea, D., Raju, T. S., Giles-Komar, J., and Feng, Y. (2012) Solubility evaluation of murine hybridoma antibodies. MAbs 4, 319-325
50. Bradbury, A. R., Sidhu, S., Dubel, S., and McCafferty, J. (2011) Beyond natural antibodies: the power of in vitro display technologies.Nat Biotechnol 29, 245-254
51. Johan, N., Peter. M. T., and Sachdev, S. S. (2016) Engineered Autonomous Human Variable Domains. Curr Pharm Des 22, 6527-6537
52. Xu, Y., Roach, W., Sun, T., Jain, T., Prinz, B., Yu, T. Y., Torrey, J., Thomas, J., Bobrowicz, P., Vasquez, M., Wittrup, K. D., and Krauland, E. (2013) Addressing polyspecificity of antibodies selected from an in vitro yeast presentation system: a FACS-based, high-throughput selection and analytical tool. Protein Eng Des Sel 26, 663-670
53. Kelly, R. L., Sun, T., Jain, T., Caffry, I., Yu, Y., Cao, Y., Lynaugh, H., Brown, M., Vasquez, M., Wittrup, K. D., and Xu, Y. (2015) High throughput cross-interaction measures for human IgGI antibodies correlate with clearance rates in mice. MAbs 7, 770-777
54. Alam, M. E., Slaney, T. R., Wu, L., Das, T. K., Kar, S., Barnett, G. V., Leone, A., and Tessier, P. M. (2020) Unique Impacts of Methionine Oxidation, Tryptophan Oxidation, and Asparagine Deamidation on Antibody Stability and Aggregation. J Pharm Sci 109, 656-669
55. Cromwell, M. E. M., Hilario, E., and Jacobson, F. (2006) Protein aggregation and bioprocessing. AAPSJ 8, E572-E579
56. Hober. S., Nord, K., and Linhult, M. (2007) Protein A chromatography for antibody purification. J Chromatogr B Analyt Technol Biomed Life Sci 848, 40-47
57. Shukla, A. A., Gupta, P., and Han, X. (2007) Protein aggregation kinetics during Protein A chromatography. Case study for an Fc fusion protein. J Chromatogr A 1171, 22-28
58. Ejima, D., Tsumoto, K., Fukada. H., Yumioka, R., Nagase, K. Arakawa, T., and Philo, J. S. (2007) Effects of acid exposure on the conformation, stability, and aggregation of monoclonal antibodies. Proteins 66, 954-962
59. Hari, S. B., Lau, H., Razinkov, V. L., Chen, S., and Latypov, R. F. (2010) Acid-induced aggregation of human monoclonal IgGI and IgG2: molecular mechanism and the effect of solution composition. Biochemistry 49, 9328-9338
60. Julian, M. C., Li, L., Garde, S., Wilen, R., and Tessier, P. M. (2017) Efficient affinity maturation of antibody variable domains requires co-selection of compensatory mutations to maintain thermodynamic stability. Sci Rep 7, 45259
61. Rabia, L. A., Desai, A. A., Jhajj, H. S., and Tessier, P. M. (2018) Understanding and overcoming trade-offs between antibody affinity, specificity, stability and solubility. Biochem Eng J 137, 365-374
62. Julian, M. C., Lec, C. C., Tiller, K. E., Rabia, L. A., Day, E. K., Schick, A. J., 3rd, and Tessier, P. M. (2015) Co-evolution of affinity and stability of grafted amyloid-motif domain antibodies. Protein Eng Des Sel 28. 339-350
63. Boswell, C. A., Tesar, D. B., Mukhyala, K., Theil, F.-P., Fielder, P. J., and Khawli, L. A. (2010) Effects of Charge on Antibody Tissue Distribution and Pharmacokinetics. Bioconjug Chem 21, 2153-2163
64. Dobson, C. L., Devine, P. W., Phillips, J. J., Higazi, D. R., Lloyd, C., Popovic, B., Arnold, J., Buchanan. A., Lewis, A., Goodman, J., van der Walle, C. F., Thornton, P., Vinall, L., Lowne. D., Aagaard, A., Olsson, L. L., Ridderstad Wollberg, A., Welsh, F., Karamanos, T. K., Pashley, C. L., Iadanza, M. G., Ranson, N. A., Ashcroft, A. E., Kippen, A. D., Vaughan, T. J., Radford, S. E., and Lowe, D. C. (2016) Engineering the surface properties of a human monoclonal antibody prevents self-association and rapid clearance in vivo. Sci Rep 6, 38644
65. Starr, C. G., and Tessier, P. M. (2019) Selecting and engineering monoclonal antibodies with drug-like specificity. Curr Opin Biotechnol 60, 119-127
66. Birtalan, S., Zhang, Y., Fellouse, F. A., Shao, L., Schaefer, G., and Sidhu, S. S. (2008) The intrinsic contributions of tyrosine, serine, glycine and arginine to the affinity and specificity of antibodies. J Mol Biol 377, 1518-1528
67. Pardridge, W. M. (2015) Blood-brain barrier drug delivery of IgG fusion proteins with a transferrin receptor monoclonal antibody. Expert Opin Drug Deliv 12, 207-222
68. Jones, A. R., and Shusta, E. V. (2007) Blood-brain barrier transport of therapeutics via receptormediation.

### Pharm Res 24, 1759-1771

69. Pardridge, W. M. (2017) Delivery of Biologics Across the Blood-Brain Barrier with Molecular Trojan Horse Technology. BioDrugs 31, 503-519
70. Chung. S., Nguyen, V., Lin, Y. L., Lafrance-Vanasse, J., Scales, S. J., Lin. K., Deng. R., Williams, K., Sperinde, G., Li, J. J., Zheng, K., Sukumaran. S., Tesar, D., Ernst, J. A., Fischer, S., Lazar, G. A., Prabhu, S., and Song, A. (2019) An in vitro FeRn- dependent transcytosis assay as a screening tool for predictive assessment of nonspecific clearance of antibody therapeutics in humans. MAbs 11, 942-955
71. Triguero, D., Buciak, J. B., Yang, J., and Pardridge, W. M. (1989) Blood-brain barrier transport of cationized immunoglobulin G: enhanced delivery compared to native protein. Proceedings of the National Academy of Sciences 86, 4761-4765
72. Pardridge, W. M., Bickel, U., Buciak, J., Yang, J., Diagne, A., and Acpinus, C. (1994) Cationization of a monoclonal antibody to the human immunodeficiency virus REV protein enhances cellular uptake but does not impair antigen binding of the antibody. Immunology letters 42, 191-195
73. Triguero, D., Buciak, J. B., Yang, J., and Pardridge, W. M. (1989) Blood-brain barrier transport of cationized immunoglobulin G: enhanced delivery compared to native protein. Proc Natl Acad Sci U SA 86, 4761-4765
74. Zago, W., Schroeter, S., Guido, T., Khan, K., Seubert, P., Yednock, T., Schenk, D., Gregg, K. M., Games, D., Bard, F., and Kinney, G. G. (2013) Vascular alterations in PDAPP mice after anti-Abeta immunotherapy: Implications for amyloid-related imaging abnormalities. Alzheimers Dement 9, S105-115
75. Barkhof. F., Daams, M., Scheltens, P., Brashear, H. R., Arrighi, H. M., Bechten, A., Morris, K., McGovern, M., and Wattjes, M. P. (2013) An MRI rating scale for amyloid-related imaging abnormalities with edema or effusion. AJNR Am J Neuroradiol 34, 1550-1555
76. Sperling, R., Salloway, S., Brooks, D. J., Tampicri, D., Barakos, J., Fox, N. C., Raskind, M., Sabbagh. M., Honig, L. S., Porsteinsson, A. P., Lieberburg, I., Arrighi, H. M., Morris. K. A., Lu. Y., Liu, E., Gregg, K. M., Brashear, H. R., Kinney, G. G., Black, R., and Grundman, M. (2012) Amyloid-related imaging abnormalities in patients with Alzheimer's disease treated with bapineuzumab: a retrospective analysis. Lancet Neurol 11, 241-249
77. Pardridge, W. M. (2016) Re-engineering therapeutic antibodies for Alzheimer's disease as bloodbrain barrier penetrating bi-specific antibodies. Expert Opin Biol Ther 16. 1455-1468
78. Sumbria, R. K., Hui, E. K., Lu, J. Z., Boado, R. J., and Pardridge, W. M. (2013) Disaggregation of amyloid plaque in brain of Alzheimer's disease transgenic mice with daily subcutaneous administration of a tetravalent bispecific antibody that targets the transferrin receptor and the Abeta amyloid peptide. Mol Pharm 10, 3507-3513
79. Niewochner, J., Bohrmann, B., Collin. L., Urich, E., Sade, H., Maier, P., Rueger, P., Stracke, J. O., Lau, W., Tissot, A. C., Loetscher, H., Ghosh, A., and Freskgard, P. O. (2014) Increased brain penetration and potency of a therapeutic antibody using a monovalent molecular shuttle. Neuron 81, 49-60
80. Zuchero, Y. J., Chen, X., Bien-Ly. N., Bumbaca. D., Tong, R. K., Gao, X., Zhang, S., Hoyte, K., Luk, W., Huntley, M. A., Phu, L., Tan, C., Kallop, D., Weimer, R. M., Lu, Y., Kirkpatrick, D. S., Ernst, J. A., Chih, B., Dennis, M. S., and Watts, R. J. (2016) Discovery of Novel Blood-Brain Barrier Targets to Enhance Brain Uptake of Therapeutic Antibodies. Neuron 89, 70-82
81. Benatuil, L., Percz, J. M., Belk, J., and Hsich, C. M. (2010) An improved yeast transformation method for the generation of very large human antibody libraries. Protein Eng Des Sel 23, 155-159
82. Bushnell, B., Rood, J., and Singer, E. (2017) BBMerge—Accurate paired shotgun read merging via overlap. PLOS One 12, e0185056
83. Cock, P. J., Antao. T., Chang. J. T., Chapman, B. A., Cox, C. J., Dalke, A., Friedberg. I., Hamelryck, T., Kauff. F., Wilczynski, B., and de Hoon, M. J. (2009) Biopython: freely available Python tools for computational molecular biology and bioinformatics. Bioinformatics 25, 1422-1423

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A monoclonal antibody directed against fibrils of amyloid beta (Aβ) peptides, or an antigen-binding fragment thereof, comprising a heavy chain variable region (VH) comprising complementarity determining regions (CDRs) HCDR1, HCDR2, and HCDR3 and a light chain variable region (VL) comprising complementarity determining regions (CDRs) LCDR1, LCDR2, and LCDR3, wherein HCDR3 comprises the amino acid sequence of SEQ ID NO: 3, and wherein:
(a) HCDR1 comprises the amino acid sequence of SEQ ID NO: 1, except that at least one amino acid residue of SEQ ID NO: 1 is replaced with a different amino acid residue;
(b) HCDR2 comprises the amino acid sequence of SEQ ID NO: 2, except that at least one amino acid residue of SEQ ID NO: 2 is replaced with a different amino acid residue;
(c) LCDR1 comprises the amino acid sequence of SEQ ID NO: 4, except that at least one amino acid residue of SEQ ID NO: 4 is replaced with a different amino acid residue;
(d) LCDR2 comprises the amino acid sequence of SEQ ID NO: 5, except that at least one amino acid residue of SEQ ID NO: 5 is replaced with a different amino acid residue;
(e) LCDR3 comprises the amino acid sequence of SEQ ID NO: 6, except that at least one amino acid residue of SEQ ID NO: 6 is replaced with a different amino acid residue; or
(f) any combination of (a)-(e).

2. The monoclonal antibody of claim 1, wherein the HCDR2 comprises the amino acid sequence of any one of SEQ ID NOs: 28-36.

3. The monoclonal antibody of claim 1 or claim 2, wherein the LCDR1 comprises the amino acid sequence of any one of SEQ ID NOs: 37-43.

4. The monoclonal antibody of any one of claims 1-3, wherein the LCDR3 comprises the amino acid sequence of any one of SEQ ID NOs: 44-47.

5. The monoclonal antibody of any one of claims 1-4, wherein the HCDR1 comprises the amino acid sequence of any one of SEQ ID NOs: 54-56.

6. The monoclonal antibody of claim 1, wherein:
(a) the heavy chain variable region comprises the amino acid sequence of SEQ ID NO: 7 or an amino acid sequence that is at least 95% identical to SEQ ID NO: 7 and the light chain variable region comprises the amino acid sequence of SEQ ID NO: 8 or an amino acid sequence that is at least 95% identical to SEQ ID NO: 8;
(b) the heavy chain variable region comprises the amino acid sequence of SEQ ID NO: 9 or an amino acid sequence that is at least 95% identical to SEQ ID NO: 9 and the light chain variable region comprises the amino acid sequence of SEQ ID NO: 10 or an amino acid sequence that is at least 95% identical to SEQ ID NO: 10;
(c) the heavy chain variable region comprises the amino acid sequence of SEQ ID NO: 11 or an amino acid sequence that is at least 95% identical to SEQ ID NO: 11 and the light chain variable region comprises the amino acid sequence of SEQ ID NO: 12 or an amino acid sequence that is at least 95% identical to SEQ ID NO: 12;
(d) the heavy chain variable region comprises the amino acid sequence of SEQ ID NO: 13 or an amino acid sequence that is at least 95% identical to SEQ ID NO: 13 and the light chain variable region comprises the amino acid sequence of SEQ ID NO: 14 or an amino acid sequence that is at least 95% identical to SEQ ID NO: 14;
(e) the heavy chain variable region comprises the amino acid sequence of SEQ ID NO: 15 or an amino acid sequence that is at least 95% identical to SEQ ID NO: 15 and the light chain variable region comprises the amino acid sequence of SEQ ID NO: 16 or an amino acid sequence that is at least 95% identical to SEQ ID NO: 16;
(f) the heavy chain variable region comprises the amino acid sequence of SEQ ID NO: 17 or an amino acid sequence that is at least 95% identical to SEQ ID NO: 17 and the light chain variable region comprises the amino acid sequence of SEQ ID NO: 18 or an amino acid sequence that is at least 95% identical to SEQ ID NO: 18;
(g) the heavy chain variable region comprises the amino acid sequence of SEQ ID NO: 19 or an amino acid sequence that is at least 95% identical to SEQ ID NO: 19 and the light chain variable region comprises the amino acid sequence of SEQ ID NO: 20 or an amino acid sequence that is at least 95% identical to SEQ ID NO: 20;
(h) the heavy chain variable region comprises the amino acid sequence of SEQ ID NO: 21 or an amino acid sequence that is at least 95% identical to SEQ ID NO: 21 and the light chain variable region comprises the amino acid sequence of SEQ ID NO: 22 or an amino acid sequence that is at least 95% identical to SEQ ID NO: 22;
(i) the heavy chain variable region comprises the amino acid sequence of SEQ ID NO: 23 or an amino acid sequence that is at least 95% identical to SEQ ID NO: 23 and the light chain variable region comprises the amino acid sequence of SEQ ID NO: 24 or an amino acid sequence that is at least 95% identical to SEQ ID NO: 24;
(j) the heavy chain variable region comprises the amino acid sequence of SEQ ID NO: 25 or an amino acid sequence that is at least 95% identical to SEQ ID NO: 25 and the light chain variable region comprises the amino acid sequence of SEQ ID NO: 26 or an amino acid sequence that is at least 95% identical to SEQ ID NO: 26;
(k) the heavy chain variable region comprises the amino acid sequence of SEQ ID NO: 19 or an amino acid sequence that is at least 95% identical to SEQ ID NO: 19 and the light chain variable region comprises the amino acid sequence of SEQ ID NO: 27 or an amino acid sequence that is at least 95% identical to SEQ ID NO: 27;
(l) the heavy chain variable region comprises the amino acid sequence of SEQ ID NO: 19 or an amino acid sequence that is at least 95% identical to SEQ ID NO: 19 and the light chain variable region comprises the amino acid sequence of SEQ ID NO: 48 or an amino acid sequence that is at least 95% identical to SEQ ID NO: 48;
(m) the heavy chain variable region comprises the amino acid sequence of SEQ ID NO: 49 or an amino acid sequence that is at least 95% identical to SEQ ID NO: 49 and the light chain variable region comprises the amino acid sequence of SEQ ID NO: 50 or an amino acid sequence that is at least 95% identical to SEQ ID NO: 50; or
(n) the heavy chain variable region comprises the amino acid sequence of SEQ ID NO: 49 or an amino acid sequence that is at least 95% identical to SEQ ID NO: 49 and the light chain variable region comprises the amino acid sequence of SEQ ID NO: 51 or an amino acid sequence that is at least 95% identical to SEQ ID NO: 51.

7. The monoclonal antibody of claim 1, wherein:
(a) the heavy chain variable region comprises the amino acid sequence of SEQ ID NO: 52 or an amino acid sequence that is at least 95% identical to SEQ ID NO: 52 and the light chain variable region comprises the amino acid sequence of SEQ ID NO: 20 or an amino acid sequence that is at least 95% identical to SEQ ID NO: 20; or
(a) the heavy chain variable region comprises the amino acid sequence of SEQ ID NO: 53 or an amino acid sequence that is at least 95% identical to SEQ ID NO: 53 and the light chain variable region comprises the amino acid sequence of SEQ ID NO: 20 or an amino acid sequence that is at least 95% identical to SEQ ID NO: 20.

8. The monoclonal antibody of any one of claims 1-7, which is a whole antibody.

9. The monoclonal antibody of any of claim 1-8, wherein:
the heavy chain comprises an amino acid sequence of any one of SEQ ID NOs: 57, 59, 61, 63, 65, 67, or 71; and
the light chain comprises an amino acid sequence of any one of SEQ ID NOs: 58, 60, 62, 64, 66, 68, or 72.

10. The monoclonal antibody of claim 8 or 9, which is an IgGI, IgG2, IgG3, or lgG4 antibody.

11. The monoclonal antibody of claim 10, which comprises an engineered Fe region.

12. The monoclonal antibody of any one of claims 1-7, which is an antigen-binding antibody fragment.

13. The monoclonal antibody of claim 12, wherein the antibody fragment is selected from F(ab′)₂, Fab′, Fab, Fv, scFv, dsFv, dAb, and a single chain binding polypeptide.

14. A composition comprising the monoclonal antibody of any one of claims 1-13 and a pharmaceutically acceptable carrier.

15. A nucleic acid sequence encoding the monoclonal antibody of any one of claims 1-13.

16. A vector comprising the nucleic acid sequence of claim 15.

17. A method of treating a neurodegenerative disease in a human, which method comprises administering an effective amount of the composition of claim 14 to a human having a neurodegenerative disease, whereupon the neurodegenerative disease is treated in the human.

18. A method of diagnosing a neurodegenerative disease in a human, which method comprises contacting a sample obtained from a human suspected of having a neurodegenerative disease with the monoclonal antibody of any one of claims 1-13, wherein binding of the monoclonal antibody to fibrils of Aβ peptides present in the sample indicates that the human has a neurodegenerative disease.

19. The method of claim 17 or 18, wherein the neurodegenerative disease is Alzheimer's disease or Parkinson's disease.

20. Use of a monoclonal antibody according to any one of claims 1-13 in a method of treating a human with a neurodegenerative disease.

21. The use of claim 20, wherein the neurodegenerative disease is Alzheimer's disease or Parkinson's disease.

22. A monoclonal antibody according to any one of claims 1-13 for use in diagnosing a neurodegenerative disease in a human.

23. The monoclonal antibody of claim 22, wherein the neurodegenerative disease is Alzheimer's disease or Parkinson's disease.


SEQUENCE LISTING (74 sequences)

SEQ ID NO: 1 - protein, 11 aa, Artificial sequence
  Synthetic
SGFNIKDTYIH

SEQ ID NO: 2 - protein, 17 aa, Artificial sequence
  Synthetic
RIYPTNGYTRYADSVKG

SEQ ID NO: 3 - protein, 21 aa, Artificial sequence
  Synthetic
ARDGYDGSYFVGYDYNDFYDY

SEQ ID NO: 4 - protein, 11 aa, Artificial sequence
  Synthetic
RASQDVNTAVA

SEQ ID NO: 5 - protein, 7 aa, Artificial sequence
  Synthetic
SASFLYS

SEQ ID NO: 6 - protein, 9 aa, Artificial sequence
  Synthetic
QQHYTTPPT

SEQ ID NO: 7 - protein, 128 aa, Artificial sequence
  Synthetic
EVQLVESGGGLVQPGGSLRLSCAASGFNIKDTYIHWVRQAPGKGLEWVARIYPNSGATRYAGSVKGRFTISADTSKNTAYLQMNSLRAEDTAVYYCARDGYDGSYFVGYDYNDFYDYWGQGTLVTVSS

SEQ ID NO: 8 - protein, 107 aa, Artificial sequence
  Synthetic
DIQMTQSPSSLSASVGDRVTITCRASQNVYAAVTWYQQKPGKAPKLLIYSASFLYSGVPSRFSGSRSGTDFTLTISSLQPEDFATYYCQQHSTYPPTFGQGTKVEIK

SEQ ID NO: 9 - protein, 128 aa, Artificial sequence; identical to SEQ ID NO: 7
  Synthetic

SEQ ID NO: 10 - protein, 107 aa, Artificial sequence
  Synthetic
DIQMTQSPSSLSASVGDRVTITCRASQNVYSAVSWYQQKPGKAPKLLIYSASFLYSGVPSRFSGSRSGTDFTLTISSLQPEDFATYYCQQHYTYPPTFGQGTKVEIK

SEQ ID NO: 11 - protein, 128 aa, Artificial sequence
  Synthetic
EVQLVESGGGLVQPGGSLRLSCAASGFNIKDTYIHWVRQAPGKGLEWVARIYPASGTTRYAGSVKGRFTISADTSKNTAYLQMNSLRAEDTAVYYCARDGYDGSYFVGYDYNDFYDYWGQGTLVTVSS

SEQ ID NO: 12 - protein, 107 aa, Artificial sequence
  Synthetic
DIQMTQSPSSLSASVGDRVTITCRASQNVYSAVSWYQQKPGKAPKLLIYSASFLYSGVPSRFSGSRSGTDFTLTISSLQPEDFATYYCQQHSTYPPTFGQGTKVEIK

SEQ ID NO: 13 - protein, 128 aa, Artificial sequence
  Synthetic
EVQLVESGGGLVQPGGSLRLSCAASGFNIKDTYIHWVRQAPGKGLEWVARIYPASGTTRYAASVKGRFTISADTSKNTAYLQMNSLRAEDTAVYYCARDGYDGSYFVGYDYNDFYDYWGQGTLVTVSS

SEQ ID NO: 14 - protein, 107 aa, Artificial sequence; identical to SEQ ID NO: 12
  Synthetic

SEQ ID NO: 15 - protein, 128 aa, Artificial sequence
  Synthetic
EVQLVESGGGLVQPGGSLRLSCAASGFNIKDTYIHWVRQAPGKGLEWVARIYPSNGYTRYAGSVKGRFTISADTSKNTAYLQMNSLRAEDTAVYYCARDGYDGSYFVGYDYNDFYDYWGQGTLVTVSS

SEQ ID NO: 16 - protein, 107 aa, Artificial sequence
  Synthetic
DIQMTQSPSSLSASVGDRVTITCRASQNVYNAVTWYQQKPGKAPKLLIYSASFLYSGVPSRFSGSRSGTDFTLTISSLQPEDFATYYCQQHNTYPPTFGQGTKVEIK

SEQ ID NO: 17 - protein, 128 aa, Artificial sequence
  Synthetic
EVQLVESGGGLVQPGGSLRLSCAASGFNIKDTYIHWVRQAPGKGLEWVARIYPASGSTRYAASVKGRFTISADTSKNTAYLQMNSLRAEDTAVYYCARDGYDGSYFVGYDYNDFYDYWGQGTLVTVSS

SEQ ID NO: 18 - protein, 107 aa, Artificial sequence
  Synthetic
DIQMTQSPSSLSASVGDRVTITCRASQNVYSAVTWYQQKPGKAPKLLIYSASFLYSGVPSRFSGSRSGTDFTLTISSLQPEDFATYYCQQHSTSPPTFGQGTKVEIK

SEQ ID NO: 19 - protein, 128 aa, Artificial sequence
  Synthetic
EVQLVESGGGLVQPGGSLRLSCAASGFNIKDTYIHWVRQAPGKGLEWVARIYPASGATRYADSVKGRFTISADTSKNTAYLQMNSLRAEDTAVYYCARDGYDGSYFVGYDYNDFYDYWGQGTLVTVSS

SEQ ID NO: 20 - protein, 107 aa, Artificial sequence
  Synthetic
DIQMTQSPSSLSASVGDRVTITCRASQNVAYAVTWYQQKPGKAPKLLIYSASFLYSGVPSRFSGSRSGTDFTLTISSLQPEDFATYYCQQHSTYPPTFGQGTKVEIK

SEQ ID NO: 21 - protein, 128 aa, Artificial sequence
  Synthetic
EVQLVESGGGLVQPGGSLRLSCAASGFNIKDTYIHWVRQAPGKGLEWVARIYPSNGYTRYAASVKGRFTISADTSKNTAYLQMNSLRAEDTAVYYCARDGYDGSYFVGYDYNDFYDYWGQGTLVTVSS

SEQ ID NO: 22 - protein, 107 aa, Artificial sequence
  Synthetic
DIQMTQSPSSLSASVGDRVTITCRASQNVAYAVSWYQQKPGKAPKLLIYSASFLYSGVPSRFSGSRSGTDFTLTISSLQPEDFATYYCQQHSTYPPTFGQGTKVEIK

SEQ ID NO: 23 - protein, 128 aa, Artificial sequence
  Synthetic
EVQLVESGGGLVQPGGSLRLSCAASGFNIKDTYIHWVRQAPGKGLEWVARIYPASGSTRYAGSVKGRFTISADTSKNTAYLQMNSLRAEDTAVYYCARDGYDGSYFVGYDYNDFYDYWGQGTLVTVSS

SEQ ID NO: 24 - protein, 107 aa, Artificial sequence
  Synthetic
DIQMTQSPSSLSASVGDRVTITCRASQNVYSAVTWYQQKPGKAPKLLIYSASFLYSGVPSRFSGSRSGTDFTLTISSLQPEDFATYYCQQHSTYPPTFGQGTKVEIK

SEQ ID NO: 25 - protein, 128 aa, Artificial sequence
  Synthetic
EVQLVESGGGLVQPGGSLRLSCAASGFNIKDTYIHWVRQAPGKGLEWVARIYPASGYTRYAASVKGRFTISADTSKNTAYLQMNSLRAEDTAVYYCARDGYDGSYFVGYDYNDFYDYWGQGTLVTVSS

SEQ ID NO: 26 - protein, 107 aa, Artificial sequence
  Synthetic
DIQMTQSPSSLSASVGDRVTITCRASQNVASAVTWYQQKPGKAPKLLIYSASFLYSGVPSRFSGSRSGTDFTLTISSLQPEDFATYYCQQHYTYPPTFGQGTKVEIK

SEQ ID NO: 27 - protein, 107 aa, Artificial sequence
  Synthetic
DIQMTQSPSSLSASVGDRVTITCRASQNVAYAVTWYQQKPGKAPKLLIYGTRYLNSGVPSRFSGSRSGTDFTLTISSLQPEDFATYYCQQHSTYPPTFGQGTKVEIK

SEQ ID NO: 28 - protein, 17 aa, Artificial sequence
  Synthetic
RIYPNSGATRYAGSVKG

SEQ ID NO: 29 - protein, 17 aa, Artificial sequence
  Synthetic
RIYPASGTTRYAGSVKG

SEQ ID NO: 30 - protein, 17 aa, Artificial sequence
  Synthetic
RIYPASGTTRYAASVKG

SEQ ID NO: 31 - protein, 17 aa, Artificial sequence
  Synthetic
RIYPSNGYTRYAGSVKG

SEQ ID NO: 32 - protein, 17 aa, Artificial sequence
  Synthetic
RIYPASGSTRYAASVKG

SEQ ID NO: 33 - protein, 17 aa, Artificial sequence
  Synthetic
RIYPASGATRYADSVKG

SEQ ID NO: 34 - protein, 17 aa, Artificial sequence
  Synthetic
RIYPSNGYTRYAASVKG

SEQ ID NO: 35 - protein, 17 aa, Artificial sequence
  Synthetic
RIYPASGSTRYAGSVKG

SEQ ID NO: 36 - protein, 17 aa, Artificial sequence
  Synthetic
RIYPASGYTRYAASVKG

SEQ ID NO: 37 - protein, 11 aa, Artificial sequence
  Synthetic
RASQNVYAAVT

SEQ ID NO: 38 - protein, 11 aa, Artificial sequence
  Synthetic
RASQNVYSAVS

SEQ ID NO: 39 - protein, 11 aa, Artificial sequence
  Synthetic
RASQNVYNAVT

SEQ ID NO: 40 - protein, 11 aa, Artificial sequence
  Synthetic
RASQNVYSAVT

SEQ ID NO: 41 - protein, 11 aa, Artificial sequence
  Synthetic
RASQNVAYAVT

SEQ ID NO: 42 - protein, 11 aa, Artificial sequence
  Synthetic
RASQNVAYAVS

SEQ ID NO: 43 - protein, 11 aa, Artificial sequence
  Synthetic
RASQNVASAVT

SEQ ID NO: 44 - protein, 9 aa, Artificial sequence
  Synthetic
QQHSTYPPT

SEQ ID NO: 45 - protein, 9 aa, Artificial sequence
  Synthetic
QQHYTYPPT

SEQ ID NO: 46 - protein, 9 aa, Artificial sequence
  Synthetic
QQHNTYPPT

SEQ ID NO: 47 - protein, 9 aa, Artificial sequence
  Synthetic
QQHSTSPPT

SEQ ID NO: 48 - protein, 107 aa, Artificial sequence
  Synthetic
DIQMTQSPSSLSASVGDRVTITCRASQNVAYAVTWYQQKPGKAPKLLIYSYNKLASGVPSRFSGSRSGTDFTLTISSLQPEDFATYYCQQHSTYPPTFGQGTKVEIK

SEQ ID NO: 49 - protein, 128 aa, Artificial sequence
  Synthetic
EVQLVESGGGLVQPGGSLRLSCAASGYNIKDTYIHWVRQAPGKGLEWVARIYPASGATRYADSVKGRFTISADTSKNTAYLQMNSLRAEDTAVYYCARDGYDGSYFVGYDYNDFYDYWGQGTLVTVSS

SEQ ID NO: 50 - protein, 107 aa, Artificial sequence
  Synthetic
DIQMTQSPSSLSASVGDRVTITCRASQNVAYAVTWYQQKPGKAPKLLIYSAIFLYSGVPSRFSGSRSGTDFTLTISSLQPEDFATYYCQQHSTYPPTFGQGTKVEIK

SEQ ID NO: 51 - protein, 107 aa, Artificial sequence
  Synthetic
DIQMTQSPSSLSASVGDRVTITCRASQNVAYAVTWYQQKPGKAPKLLIYSSSFLYSGVPSRFSGSRSGTDFTLTISSLQPEDFATYYCQQHSTYPPTFGQGTKVEIK

SEQ ID NO: 52 - protein, 128 aa, Artificial sequence
  Synthetic
EVQLVESGGGLVQPGGSLRLSCAASGYNIKATYIHWVRQAPGKGLEWVARIYPASGATRYADSVKGRFTISADTSKNTAYLQMNSLRAEDTAVYYCARDGYDGSYFVGYDYNDFYDYWGQGTLVTVSS

SEQ ID NO: 53 - protein, 128 aa, Artificial sequence
  Synthetic
EVQLVESGGGLVQPGGSLRLSCAASGFNIKGTYSHWVRQAPGKGLEWVARIYPASGATRYADSVKGRFTISADTSKNTAYLQMNSLRAEDTAVYYCARDGYDGSYFVGYDYNDFYDYWGQGTLVTVSS

SEQ ID NO: 54 - protein, 11 aa, Artificial sequence
  Synthetic
SGYNIKATYIH

SEQ ID NO: 55 - protein, 11 aa, Artificial sequence
  Synthetic
SGFNIKGTYSH

SEQ ID NO: 56 - protein, 11 aa, Artificial sequence
  Synthetic
SGYNIKDTYIH

SEQ ID NO: 57 - protein, 458 aa, Artificial sequence
  Synthetic
EVQLVESGGGLVQPGGSLRLSCAASGFNIKDTYIHWVRQAPGKGLEWVARIYPASGATRYADSVKGRFTISADTSKNTAYLQMNSLRAEDTAVYYCARDGYDGSYFVGYDYNDFYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, Artificial sequence
  Synthetic
DIQMTQSPSSLSASVGDRVTITCRASQNVAYAVTWYQQKPGKAPKLLIYSASFLYSGVPSRFSGSRSGTDFTLTISSLQPEDFATYYCQQHSTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 458 aa, Artificial sequence
  Synthetic
EVQLVESGGGLVQPGGSLRLSCAASGYNIKATYIHWVRQAPGKGLEWVARIYPASGATRYADSVKGRFTISADTSKNTAYLQMNSLRAEDTAVYYCARDGYDGSYFVGYDYNDFYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Artificial sequence; identical to SEQ ID NO: 58
  Synthetic

SEQ ID NO: 61 - protein, 458 aa, Artificial sequence
  Synthetic
EVQLVESGGGLVQPGGSLRLSCAASGYNIKDTYIHWVRQAPGKGLEWVARIYPASGATRYADSVKGRFTISADTSKNTAYLQMNSLRAEDTAVYYCARDGYDGSYFVGYDYNDFYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, Artificial sequence
  Synthetic
DIQMTQSPSSLSASVGDRVTITCRASQNVAYAVTWYQQKPGKAPKLLIYSAIFLYSGVPSRFSGSRSGTDFTLTISSLQPEDFATYYCQQHSTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 458 aa, Artificial sequence; identical to SEQ ID NO: 61
  Synthetic

SEQ ID NO: 64 - protein, 214 aa, Artificial sequence
  Synthetic
DIQMTQSPSSLSASVGDRVTITCRASQNVAYAVTWYQQKPGKAPKLLIYSSSFLYSGVPSRFSGSRSGTDFTLTISSLQPEDFATYYCQQHSTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 458 aa, Artificial sequence
  Synthetic
EVQLVESGGGLVQPGGSLRLSCAASGFNIKGTYSHWVRQAPGKGLEWVARIYPASGATRYADSVKGRFTISADTSKNTAYLQMNSLRAEDTAVYYCARDGYDGSYFVGYDYNDFYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, Artificial sequence; identical to SEQ ID NO: 58
  Synthetic

SEQ ID NO: 67 - protein, 458 aa, Artificial sequence; identical to SEQ ID NO: 57
  Synthetic

SEQ ID NO: 68 - protein, 214 aa, Artificial sequence
  Synthetic
DIQMTQSPSSLSASVGDRVTITCRASQNVAYAVTWYQQKPGKAPKLLIYGTRYLNSGVPSRFSGSRSGTDFTLTISSLQPEDFATYYCQQHSTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 458 aa, Artificial sequence; identical to SEQ ID NO: 59
  Synthetic

SEQ ID NO: 70 - protein, 214 aa, Artificial sequence; identical to SEQ ID NO: 58
  Synthetic

SEQ ID NO: 71 - protein, 458 aa, Artificial sequence; identical to SEQ ID NO: 57
  Synthetic

SEQ ID NO: 72 - protein, 214 aa, Artificial sequence
  Synthetic
DIQMTQSPSSLSASVGDRVTITCRASQNVAYAVTWYQQKPGKAPKLLIYSYNKLASGVPSRFSGSRSGTDFTLTISSLQPEDFATYYCQQHSTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 5 aa, Artificial sequence
  Synthetic
YTNYD

SEQ ID NO: 74 - protein, 4 aa, Artificial sequence
  Synthetic
DNTA